SEQUENCE LISTING (434 sequences)

SEQ ID NO: 1 - DNA, 15 bp, Artificial Sequence
  Oligonucleotide
gctagacgttagcgt

SEQ ID NO: 2 - DNA, 15 bp, Artificial Sequence
  Oligonucleotide
gctagatgttagcgt

SEQ ID NO: 3 - DNA, 15 bp, Artificial Sequence
  Oligonucleotide
gctagacgttagcgt

SEQ ID NO: 4 - DNA, 15 bp, Artificial Sequence
  Oligonucleotide
gctagacgttagcgt

SEQ ID NO: 5 - DNA, 15 bp, Artificial Sequence
  Oligonucleotide
gcatgacgttgagct

SEQ ID NO: 6 - DNA, 20 bp, Artificial Sequence
  Oligonucleotide
atggaaggtccagcgttctc

SEQ ID NO: 7 - DNA, 20 bp, Artificial Sequence
  Oligonucleotide
atcgactctcgagcgttctc

SEQ ID NO: 8 - DNA, 20 bp, Artificial Sequence
  Oligonucleotide
atcgactctcgagcgttctc

SEQ ID NO: 9 - DNA, 20 bp, Artificial Sequence
  Oligonucleotide
atcgactctcgagcgttctc

SEQ ID NO: 10 - DNA, 20 bp, Artificial Sequence
  Oligonucleotide
atggaaggtccaacgttctc

SEQ ID NO: 11 - DNA, 20 bp, Artificial Sequence
  Oligonucleotide
gagaacgctggaccttccat

SEQ ID NO: 12 - DNA, 20 bp, Artificial Sequence
  Oligonucleotide
gagaacgctcgaccttccat

SEQ ID NO: 13 - DNA, 20 bp, Artificial Sequence
  Oligonucleotide
gagaacgctcgaccttcgat

SEQ ID NO: 14 - DNA, 20 bp, Artificial Sequence
  Oligonucleotide
gagaacgctggaccttccat

SEQ ID NO: 15 - DNA, 20 bp, Artificial Sequence
  Oligonucleotide
gagaacgatggaccttccat

SEQ ID NO: 16 - DNA, 20 bp, Artificial Sequence
  Oligonucleotide
gagaacgctccagcactgat

SEQ ID NO: 17 - DNA, 20 bp, Artificial Sequence
  Oligonucleotide
tccatgtcggtcctgatgct

SEQ ID NO: 18 - DNA, 20 bp, Artificial Sequence
  Oligonucleotide
tccatgtcggtcctgatgct

SEQ ID NO: 19 - DNA, 20 bp, Artificial Sequence
  Oligonucleotide
tccatgacgttcctgatgct

SEQ ID NO: 20 - DNA, 20 bp, Artificial Sequence
  Oligonucleotide
tccatgtcggtcctgctgat

SEQ ID NO: 21 - DNA, 8 bp, Artificial Sequence
  Oligonucleotide
tcaacgtt

SEQ ID NO: 22 - DNA, 8 bp, Artificial Sequence
  Oligonucleotide
tcagcgct

SEQ ID NO: 23 - DNA, 8 bp, Artificial Sequence
  Oligonucleotide
tcatcgat

SEQ ID NO: 24 - DNA, 8 bp, Artificial Sequence
  Oligonucleotide
tcttcgaa

SEQ ID NO: 25 - DNA, 7 bp, Artificial Sequence
  Oligonucleotide
caacgtt

SEQ ID NO: 26 - DNA, 8 bp, Artificial Sequence
  Oligonucleotide
ccaacgtt

SEQ ID NO: 27 - DNA, 8 bp, Artificial Sequence
  Oligonucleotide
aacgttct

SEQ ID NO: 28 - DNA, 8 bp, Artificial Sequence
  Oligonucleotide
tcaacgtc

SEQ ID NO: 29 - DNA, 20 bp, Artificial Sequence
  Oligonucleotide
atggactctccagcgttctc

SEQ ID NO: 30 - DNA, 20 bp, Artificial Sequence
  Oligonucleotide
atggaaggtccaacgttctc

SEQ ID NO: 31 - DNA, 20 bp, Artificial Sequence
  Oligonucleotide
atcgactctcgagcgttctc

SEQ ID NO: 32 - DNA, 20 bp, Artificial Sequence
  Oligonucleotide
atggaggctccatcgttctc

SEQ ID NO: 33 - DNA, 20 bp, Artificial Sequence
  Oligonucleotide
atcgactctcgagcgttctc

SEQ ID NO: 34 - DNA, 20 bp, Artificial Sequence
  Oligonucleotide
atcgactctcgagcgttctc

SEQ ID NO: 35 - DNA, 20 bp, Artificial Sequence
  Oligonucleotide
tccatgtcggtcctgatgct

SEQ ID NO: 36 - DNA, 20 bp, Artificial Sequence
  Oligonucleotide
tccatgccggtcctgatgct

SEQ ID NO: 37 - DNA, 20 bp, Artificial Sequence
  Oligonucleotide
tccatggcggtcctgatgct

SEQ ID NO: 38 - DNA, 20 bp, Artificial Sequence
  Oligonucleotide
tccatgacggtcctgatgct

SEQ ID NO: 39 - DNA, 20 bp, Artificial Sequence
  Oligonucleotide
tccatgtcgatcctgatgct

SEQ ID NO: 40 - DNA, 20 bp, Artificial Sequence
  Oligonucleotide
tccatgtcgctcctgatgct

SEQ ID NO: 41 - DNA, 20 bp, Artificial Sequence
  Oligonucleotide
tccatgtcgtccctgatgct

SEQ ID NO: 42 - DNA, 20 bp, Artificial Sequence
  Oligonucleotide
tccatgacgtgcctgatgct

SEQ ID NO: 43 - DNA, 20 bp, Artificial Sequence
  Oligonucleotide
tccataacgttcctgatgct

SEQ ID NO: 44 - DNA, 20 bp, Artificial Sequence
  Oligonucleotide
tccatgacgtccctgatgct

SEQ ID NO: 45 - DNA, 20 bp, Artificial Sequence
  Oligonucleotide
tccatcacgtgcctgatgct

SEQ ID NO: 46 - DNA, 19 bp, Artificial Sequence
  Oligonucleotide
ggggtcaacgttgacgggg

SEQ ID NO: 47 - DNA, 19 bp, Artificial Sequence
  Oligonucleotide
ggggtcagtcgtgacgggg

SEQ ID NO: 48 - DNA, 15 bp, Artificial Sequence
  Oligonucleotide
gctagacgttagtgt

SEQ ID NO: 49 - DNA, 20 bp, Artificial Sequence
  Oligonucleotide
tccatgtcgttcctgatgct

SEQ ID NO: 50 - DNA, 24 bp, Artificial Sequence
  Oligonucleotide
accatggacgatctgtttcccctc

SEQ ID NO: 51 - DNA, 18 bp, Artificial Sequence
  Oligonucleotide
tctcccagcgtgcgccat

SEQ ID NO: 52 - DNA, 24 bp, Artificial Sequence
  Oligonucleotide
accatggacgaactgtttcccctc

SEQ ID NO: 53 - DNA, 24 bp, Artificial Sequence
  Oligonucleotide
accatggacgagctgtttcccctc

SEQ ID NO: 54 - DNA, 24 bp, Artificial Sequence
  Oligonucleotide
accatggacgacctgtttcccctc

SEQ ID NO: 55 - DNA, 24 bp, Artificial Sequence
  Oligonucleotide
accatggacgtactgtttcccctc

SEQ ID NO: 56 - DNA, 24 bp, Artificial Sequence
  Oligonucleotide
accatggacggtctgtttcccctc

SEQ ID NO: 57 - DNA, 24 bp, Artificial Sequence
  Oligonucleotide
accatggacgttctgtttcccctc

SEQ ID NO: 58 - DNA, 15 bp, Artificial Sequence
  Oligonucleotide
cacgttgaggggcat

SEQ ID NO: 59 - DNA, 12 bp, Artificial Sequence
  Oligonucleotide
tcagcgtgcgcc

SEQ ID NO: 60 - DNA, 17 bp, Artificial Sequence
  Oligonucleotide
atgacgttcctgacgtt

SEQ ID NO: 61 - DNA, 17 bp, Artificial Sequence
  Oligonucleotide
tctcccagcgggcgcat

SEQ ID NO: 62 - DNA, 20 bp, Artificial Sequence
  Oligonucleotide
tccatgtcgttcctgtcgtt

SEQ ID NO: 63 - DNA, 20 bp, Artificial Sequence
  Oligonucleotide
tccatagcgttcctagcgtt

SEQ ID NO: 64 - DNA, 21 bp, Artificial Sequence
  Oligonucleotide
tcgtcgctgtctccccttctt

SEQ ID NO: 65 - DNA, 19 bp, Artificial Sequence
  Oligonucleotide
tcctgacgttcctgacgtt

SEQ ID NO: 66 - DNA, 19 bp, Artificial Sequence
  Oligonucleotide
tcctgtcgttcctgtcgtt

SEQ ID NO: 67 - DNA, 20 bp, Artificial Sequence
  Oligonucleotide
tccatgtcgtttttgtcgtt

SEQ ID NO: 68 - DNA, 20 bp, Artificial Sequence
  Oligonucleotide
tcctgtcgttccttgtcgtt

SEQ ID NO: 69 - DNA, 20 bp, Artificial Sequence
  Oligonucleotide
tccttgtcgttcctgtcgtt

SEQ ID NO: 70 - DNA, 20 bp, Artificial Sequence
  Oligonucleotide
tcctgtcgttttttgtcgtt

SEQ ID NO: 71 - DNA, 21 bp, Artificial Sequence
  Oligonucleotide
tcgtcgctgtctgcccttctt

SEQ ID NO: 72 - DNA, 21 bp, Artificial Sequence
  Oligonucleotide
tcgtcgctgttgtcgtttctt

SEQ ID NO: 73 - DNA, 20 bp, Artificial Sequence
  Oligonucleotide
tccatgcgtgcgtgcgtttt

SEQ ID NO: 74 - DNA, 20 bp, Artificial Sequence
  Oligonucleotide
tccatgcgttgcgttgcgtt

SEQ ID NO: 75 - DNA, 20 bp, Artificial Sequence
  Oligonucleotide
tccacgacgttttcgacgtt

SEQ ID NO: 76 - DNA, 20 bp, Artificial Sequence
  Oligonucleotide
tcgtcgttgtcgttgtcgtt

SEQ ID NO: 77 - DNA, 24 bp, Artificial Sequence
  Oligonucleotide
tcgtcgttttgtcgttttgtcgtt

SEQ ID NO: 78 - DNA, 22 bp, Artificial Sequence
  Oligonucleotide
tcgtcgttgtcgttttgtcgtt

SEQ ID NO: 79 - DNA, 21 bp, Artificial Sequence
  Oligonucleotide
gcgtgcgttgtcgttgtcgtt

SEQ ID NO: 80 - DNA, 21 bp, Artificial Sequence
  Oligonucleotide
tgtcgtttgtcgtttgtcgtt

SEQ ID NO: 81 - DNA, 25 bp, Artificial Sequence
  Oligonucleotide
tgtcgttgtcgttgtcgttgtcgtt

SEQ ID NO: 82 - DNA, 19 bp, Artificial Sequence
  Oligonucleotide
tgtcgttgtcgttgtcgtt

SEQ ID NO: 83 - DNA, 14 bp, Artificial Sequence
  Oligonucleotide
tcgtcgtcgtcgtt

SEQ ID NO: 84 - DNA, 13 bp, Artificial Sequence
  Oligonucleotide
tgtcgttgtcgtt

SEQ ID NO: 85 - DNA, 20 bp, Artificial Sequence
  Oligonucleotide
tccatagcgttcctagcgtt

SEQ ID NO: 86 - DNA, 20 bp, Artificial Sequence
  Oligonucleotide
tccatgacgttcctgacgtt

SEQ ID NO: 87 - DNA, 6 bp, Artificial Sequence
  Oligonucleotide
gtcgnt

SEQ ID NO: 88 - DNA, 7 bp, Artificial Sequence
  Oligonucleotide
tgtcgnt

SEQ ID NO: 89 - DNA, 18 bp, Artificial Sequence
  Oligonucleotide
agctatgacgttccaagg

SEQ ID NO: 90 - DNA, 20 bp, Artificial Sequence
  Oligonucleotide
tccatgacgttcctgacgtt

SEQ ID NO: 91 - DNA, 20 bp, Artificial Sequence
  Oligonucleotide
atcgactctcgaacgttctc

SEQ ID NO: 92 - DNA, 20 bp, Artificial Sequence
  Oligonucleotide
tccatgtcggtcctgacgca

SEQ ID NO: 93 - DNA, 8 bp, Artificial Sequence
  Oligonucleotide
tcttcgat

SEQ ID NO: 94 - DNA, 20 bp, Artificial Sequence
  Oligonucleotide
ataggaggtccaacgttctc

SEQ ID NO: 95 - DNA, 15 bp, Artificial Sequence
  Oligonucleotide
gctagaggggagggt

SEQ ID NO: 96 - DNA, 15 bp, Artificial Sequence
  Oligonucleotide
gctagatgttagggg

SEQ ID NO: 97 - DNA, 15 bp, Artificial Sequence
  Oligonucleotide
gctagaggggagggt

SEQ ID NO: 98 - DNA, 15 bp, Artificial Sequence
  Oligonucleotide
gctagaggggagggt

SEQ ID NO: 99 - DNA, 15 bp, Artificial Sequence
  Oligonucleotide
gcatgagggggagct

SEQ ID NO: 100 - DNA, 20 bp, Artificial Sequence
  Oligonucleotide
atggaaggtccagggggctc

SEQ ID NO: 101 - DNA, 20 bp, Artificial Sequence
  Oligonucleotide
atggactctggagggggctc

SEQ ID NO: 102 - DNA, 20 bp, Artificial Sequence
  Oligonucleotide
atggactctggagggggctc

SEQ ID NO: 103 - DNA, 20 bp, Artificial Sequence
  Oligonucleotide
atggactctggagggggctc

SEQ ID NO: 104 - DNA, 20 bp, Artificial Sequence
  Oligonucleotide
atggaaggtccaaggggctc

SEQ ID NO: 105 - DNA, 20 bp, Artificial Sequence
  Oligonucleotide
gagaaggggggaccttccat

SEQ ID NO: 106 - DNA, 20 bp, Artificial Sequence
  Oligonucleotide
gagaaggggggaccttccat

SEQ ID NO: 107 - DNA, 20 bp, Artificial Sequence
  Oligonucleotide
gagaaggggggaccttggat

SEQ ID NO: 108 - DNA, 20 bp, Artificial Sequence
  Oligonucleotide
gagaaggggggaccttccat

SEQ ID NO: 109 - DNA, 20 bp, Artificial Sequence
  Oligonucleotide
gagaaggggggaccttccat

SEQ ID NO: 110 - DNA, 20 bp, Artificial Sequence
  Oligonucleotide
gagaaggggccagcactgat

SEQ ID NO: 111 - DNA, 20 bp, Artificial Sequence
  Oligonucleotide
tccatgtggggcctgatgct

SEQ ID NO: 112 - DNA, 20 bp, Artificial Sequence
  Oligonucleotide
tccatgtggggcctgatgct

SEQ ID NO: 113 - DNA, 20 bp, Artificial Sequence
  Oligonucleotide
tccatgaggggcctgatgct

SEQ ID NO: 114 - DNA, 20 bp, Artificial Sequence
  Oligonucleotide
tccatgtggggcctgctgat

SEQ ID NO: 115 - DNA, 20 bp, Artificial Sequence
  Oligonucleotide
atggactctccggggttctc

SEQ ID NO: 116 - DNA, 20 bp, Artificial Sequence
  Oligonucleotide
atggaaggtccggggttctc

SEQ ID NO: 117 - DNA, 20 bp, Artificial Sequence
  Oligonucleotide
atggactctggaggggtctc

SEQ ID NO: 118 - DNA, 20 bp, Artificial Sequence
  Oligonucleotide
atggaggctccatggggctc

SEQ ID NO: 119 - DNA, 20 bp, Artificial Sequence
  Oligonucleotide
atggactctggggggttctc

SEQ ID NO: 120 - DNA, 20 bp, Artificial Sequence
  Oligonucleotide
atggactctggggggttctc

SEQ ID NO: 121 - DNA, 20 bp, Artificial Sequence
  Oligonucleotide
tccatgtgggtggggatgct

SEQ ID NO: 122 - DNA, 20 bp, Artificial Sequence
  Oligonucleotide
tccatgcgggtggggatgct

SEQ ID NO: 123 - DNA, 20 bp, Artificial Sequence
  Oligonucleotide
tccatgggggtcctgatgct

SEQ ID NO: 124 - DNA, 20 bp, Artificial Sequence
  Oligonucleotide
tccatgggggtcctgatgct

SEQ ID NO: 125 - DNA, 20 bp, Artificial Sequence
  Oligonucleotide
tccatgtggggcctgatgct

SEQ ID NO: 126 - DNA, 20 bp, Artificial Sequence
  Oligonucleotide
tccatgtggggcctgatgct

SEQ ID NO: 127 - DNA, 20 bp, Artificial Sequence
  Oligonucleotide
tccatggggtccctgatgct

SEQ ID NO: 128 - DNA, 20 bp, Artificial Sequence
  Oligonucleotide
tccatggggtgcctgatgct

SEQ ID NO: 129 - DNA, 20 bp, Artificial Sequence
  Oligonucleotide
tccatggggttcctgatgct

SEQ ID NO: 130 - DNA, 20 bp, Artificial Sequence
  Oligonucleotide
tccatggggtccctgatgct

SEQ ID NO: 131 - DNA, 20 bp, Artificial Sequence
  Oligonucleotide
tccatcgggggcctgatgct

SEQ ID NO: 132 - DNA, 14 bp, Artificial Sequence
  Oligonucleotide
gctagagggagtgt

SEQ ID NO: 133 - DNA, 20 bp, Artificial Sequence
  Oligonucleotide
gggggggggggggggggggg

SEQ ID NO: 134 - DNA, 21 bp, Artificial Sequence
  Oligonucleotide
actgacagactgacagactga

SEQ ID NO: 135 - DNA, 21 bp, Artificial Sequence
  Oligonucleotide
agtgacagacagacacactga

SEQ ID NO: 136 - DNA, 21 bp, Artificial Sequence
  Oligonucleotide
actgacagactgatagaccca

SEQ ID NO: 137 - DNA, 21 bp, Artificial Sequence
  Oligonucleotide
agtgagagactgcaagactga

SEQ ID NO: 138 - DNA, 21 bp, Artificial Sequence
  Oligonucleotide
aatgccagtccgacaggctga

SEQ ID NO: 139 - DNA, 21 bp, Artificial Sequence
  Oligonucleotide
ccagaacagaagcaatggatg

SEQ ID NO: 140 - DNA, 21 bp, Artificial Sequence
  Oligonucleotide
cctgaacagaagccatggatg

SEQ ID NO: 141 - DNA, 21 bp, Artificial Sequence
  Oligonucleotide
gcagaacagaagacatggatg

SEQ ID NO: 142 - DNA, 21 bp, Artificial Sequence
  Oligonucleotide
ccacaacacaagcaatggata

SEQ ID NO: 143 - DNA, 21 bp, Artificial Sequence
  Oligonucleotide
aagctagccagctagctagca

SEQ ID NO: 144 - DNA, 21 bp, Artificial Sequence
  Oligonucleotide
cagctagccacctagctagca

SEQ ID NO: 145 - DNA, 21 bp, Artificial Sequence
  Oligonucleotide
aagctaggcagctaactagca

SEQ ID NO: 146 - DNA, 21 bp, Artificial Sequence
  Oligonucleotide
gagctagcaagctagctagga

SEQ ID NO: 147 - DNA, 24 bp, Artificial Sequence
  Oligonucleotide
tcgtcgttttgtcgttttgtcgtt

SEQ ID NO: 148 - DNA, 24 bp, Artificial Sequence
  Oligonucleotide
tcgtcgtttcgtcgtttcgtcgtt

SEQ ID NO: 149 - DNA, 21 bp, Artificial Sequence
  Oligonucleotide
tcgtcgtttttcggtcgtttt

SEQ ID NO: 150 - DNA, 24 bp, Artificial Sequence
  Oligonucleotide
tcgtcgtttcgtcgttttgtcgtt

SEQ ID NO: 151 - DNA, 24 bp, Artificial Sequence
  Oligonucleotide
tcgtcgttttgtcgtttttttcga

SEQ ID NO: 152 - DNA, 22 bp, Artificial Sequence
  Oligonucleotide
tcgtcgtttttcgtgcgttttt

SEQ ID NO: 153 - DNA, 22 bp, Artificial Sequence
  Oligonucleotide
tcgtcgttgtcgttttgtcgtt

SEQ ID NO: 154 - DNA, 27 bp, Artificial Sequence
  Oligonucleotide
tcgcgtgcgttttgtcgttttgacgtt

SEQ ID NO: 155 - DNA, 23 bp, Artificial Sequence
  Oligonucleotide
tcgtcgtttgtcgttttgtcgtt

SEQ ID NO: 156 - DNA, 20 bp, Artificial Sequence
  Oligonucleotide
gggggacgatcgtcgggggg

SEQ ID NO: 157 - DNA, 10 bp, Artificial Sequence
  Oligonucleotide
tcntnncgnn

SEQ ID NO: 158 - DNA, 12 bp, Artificial Sequence
  Oligonucleotide
cgacgttcgtcg

SEQ ID NO: 159 - DNA, 13 bp, Artificial Sequence
  Oligonucleotide
cggcgccgtgccg

SEQ ID NO: 160 - DNA, 12 bp, Artificial Sequence
  Oligonucleotide
ccccccgggggg

SEQ ID NO: 161 - DNA, 12 bp, Artificial Sequence
  Oligonucleotide
ggggggcccccc

SEQ ID NO: 162 - DNA, 10 bp, Artificial Sequence
  Oligonucleotide
cccccggggg

SEQ ID NO: 163 - DNA, 10 bp, Artificial Sequence
  Oligonucleotide
gggggccccc

SEQ ID NO: 164 - DNA, 22 bp, Artificial Sequence
  Oligonucleotide
tcgtcgttttcggcgcgcgccg

SEQ ID NO: 165 - DNA, 22 bp, Artificial Sequence
  Oligonucleotide
tcgtcgttttcggcggccgccg

SEQ ID NO: 166 - DNA, 22 bp, Artificial Sequence
  Oligonucleotide
tcgtcgttttcggcgcgccgcg

SEQ ID NO: 167 - DNA, 22 bp, Artificial Sequence
  Oligonucleotide
tcgtcgttttcggcgccggccg

SEQ ID NO: 168 - DNA, 22 bp, Artificial Sequence
  Oligonucleotide
tcgtcgttttcggcccgcgcgg

SEQ ID NO: 169 - DNA, 27 bp, Artificial Sequence
  Oligonucleotide
tcgtcgttttcggcgcgcgccgttttt

SEQ ID NO: 170 - DNA, 22 bp, Artificial Sequence
  Oligonucleotide
tcctgacgttcggcgcgcgccg

SEQ ID NO: 171 - DNA, 22 bp, Artificial Sequence
  Oligonucleotide
tngtngttttnggngngngnng

SEQ ID NO: 172 - DNA, 22 bp, Artificial Sequence
  Oligonucleotide
tcctgacgttcggcgcgcgccc

SEQ ID NO: 173 - DNA, 22 bp, Artificial Sequence
  Oligonucleotide
tcgtcgttttcggcggccgacg

SEQ ID NO: 174 - DNA, 22 bp, Artificial Sequence
  Oligonucleotide
tcgtcgttttcgtcggccgccg

SEQ ID NO: 175 - DNA, 22 bp, Artificial Sequence
  Oligonucleotide
tcgtcgttttcgacggccgccg

SEQ ID NO: 176 - DNA, 22 bp, Artificial Sequence
  Oligonucleotide
tcgtcgttttcggcggccgtcg

SEQ ID NO: 177 - DNA, 22 bp, Artificial Sequence
  Oligonucleotide
tcggcgcgcgccgtcgtcgttt

SEQ ID NO: 178 - DNA, 21 bp, Artificial Sequence
  Oligonucleotide
tcgtcgtttcgacggccgtcg

SEQ ID NO: 179 - DNA, 21 bp, Artificial Sequence
  Oligonucleotide
tcgtcgtttcgacgatcgtcg

SEQ ID NO: 180 - DNA, 21 bp, Artificial Sequence
  Oligonucleotide
tcgtcgtttcgacgtacgtcg

SEQ ID NO: 181 - DNA, 18 bp, Artificial Sequence
  Oligonucleotide
tcgtcgcgacggccgtcg

SEQ ID NO: 182 - DNA, 18 bp, Artificial Sequence
  Oligonucleotide
tcgtcgcgacgatcgtcg

SEQ ID NO: 183 - DNA, 18 bp, Artificial Sequence
  Oligonucleotide
tcgtcgcgacgtacgtcg

SEQ ID NO: 184 - DNA, 22 bp, Artificial Sequence
  Oligonucleotide
tcgtttttttcgacggccgtcg

SEQ ID NO: 185 - DNA, 22 bp, Artificial Sequence
  Oligonucleotide
tcgtttttttcgacgatcgtcg

SEQ ID NO: 186 - DNA, 22 bp, Artificial Sequence
  Oligonucleotide
tcgtttttttcgacgtacgtcg

SEQ ID NO: 187 - DNA, 22 bp, Artificial Sequence
  Oligonucleotide
tngtngttttcggcggccgccg

SEQ ID NO: 188 - DNA, 22 bp, Artificial Sequence
  Oligonucleotide
tcntcnttttcggcggccgccg

SEQ ID NO: 189 - DNA, 17 bp, Artificial Sequence
  Oligonucleotide
acgtcgttttcgtcgtt

SEQ ID NO: 190 - DNA, 16 bp, Artificial Sequence
  Oligonucleotide
gcgtcgacgtcgacgc

SEQ ID NO: 191 - DNA, 16 bp, Artificial Sequence
  Oligonucleotide
gcgtcgttttcgtcgc

SEQ ID NO: 192 - DNA, 19 bp, Artificial Sequence
  Oligonucleotide
tccatgacgttcctgatgc

SEQ ID NO: 193 - DNA, 17 bp, Artificial Sequence
  Oligonucleotide
tcgtcgttttcgtcgtt

SEQ ID NO: 194 - DNA, 22 bp, Artificial Sequence
  Oligonucleotide
tcgtcgttttcggcggccgccg

SEQ ID NO: 195 - DNA, 17 bp, Artificial Sequence
  Oligonucleotide
tcgtcgttttcgtcgtt

SEQ ID NO: 196 - DNA, 16 bp, Artificial Sequence
  Oligonucleotide
tcgtcgtttcgtcgtt

SEQ ID NO: 197 - DNA, 17 bp, Artificial Sequence
  Oligonucleotide
tcgtcgttttcgtcgtt

SEQ ID NO: 198 - DNA, 17 bp, Artificial Sequence
  Oligonucleotide
tcgtcgttttcgtcgtt

SEQ ID NO: 199 - DNA, 17 bp, Artificial Sequence
  Oligonucleotide
tcgtcgttttcgtcgtt

SEQ ID NO: 200 - DNA, 24 bp, Artificial Sequence
  Oligonucleotide
tcntcnttttgtcgttttgtcgtt

SEQ ID NO: 201 - DNA, 24 bp, Artificial Sequence
  Oligonucleotide
tcntcgttttgtcgttttgtcntt

SEQ ID NO: 202 - DNA, 22 bp, Artificial Sequence
  Oligonucleotide
tcgccgttttcggcggccgccg

SEQ ID NO: 203 - DNA, 21 bp, Artificial Sequence
  Oligonucleotide
tcgtcgttttacgacgtcgcg

SEQ ID NO: 204 - DNA, 21 bp, Artificial Sequence
  Oligonucleotide
tcgtcgttttacgacgtcgtg

SEQ ID NO: 205 - DNA, 24 bp, Artificial Sequence
  Oligonucleotide
tcgtcgttttacggcgccgcgccg

SEQ ID NO: 206 - DNA, 21 bp, Artificial Sequence
  Oligonucleotide
tcgtcgttttacggcgtcgcg

SEQ ID NO: 207 - DNA, 24 bp, Artificial Sequence
  Oligonucleotide
tcgtcgttttacggcgtcgcgccg

SEQ ID NO: 208 - DNA, 24 bp, Artificial Sequence
  Oligonucleotide
tcgtcgttttacggcgtcgtgccg

SEQ ID NO: 209 - DNA, 22 bp, Artificial Sequence
  Oligonucleotide
tcgtcgttttcggcgcgcgccg

SEQ ID NO: 210 - DNA, 17 bp, Artificial Sequence
  Oligonucleotide
tcgtcgttttcgtcgtt

SEQ ID NO: 211 - DNA, 17 bp, Artificial Sequence
  Oligonucleotide
tcgtcgttttcgtcgtt

SEQ ID NO: 212 - DNA, 17 bp, Artificial Sequence
  Oligonucleotide
tcgtcgttttcgtcgtt

SEQ ID NO: 213 - DNA, 21 bp, Artificial Sequence
  Oligonucleotide
tcgtcgttttgcgacgtcgcg

SEQ ID NO: 214 - DNA, 21 bp, Artificial Sequence
  Oligonucleotide
tcgtcgtttttcgacgtcgag

SEQ ID NO: 215 - DNA, 21 bp, Artificial Sequence
  Oligonucleotide
tcgtcgtttttcgacgtcgcg

SEQ ID NO: 216 - DNA, 24 bp, Artificial Sequence
  Oligonucleotide
tcgtcnttttgtcgttttntcgtt

SEQ ID NO: 217 - DNA, 16 bp, Artificial Sequence
  Oligonucleotide
tcgtcgtttcgacgtt

SEQ ID NO: 218 - DNA, 24 bp, Artificial Sequence
  Oligonucleotide
tcgtcgtttcgacgttttgtcgtt

SEQ ID NO: 219 - DNA, 28 bp, Artificial Sequence
  Oligonucleotide
tcgtcgtttcgtcgacgtcgtttcgtcg

SEQ ID NO: 220 - DNA, 16 bp, Artificial Sequence
  Oligonucleotide
tcgtcgtttcgtcgat

SEQ ID NO: 221 - DNA, 17 bp, Artificial Sequence
  Oligonucleotide
tcgtcgtttcgtcgatt

SEQ ID NO: 222 - DNA, 15 bp, Artificial Sequence
  Oligonucleotide
tcgtcgtttcgtcgt

SEQ ID NO: 223 - DNA, 16 bp, Artificial Sequence
  Oligonucleotide
tcgtcgtttcgtcgtt

SEQ ID NO: 224 - DNA, 24 bp, Artificial Sequence
  Oligonucleotide
tcgtcgtttcgtcgtttcgtcgtt

SEQ ID NO: 225 - DNA, 24 bp, Artificial Sequence
  Oligonucleotide
tcgtcgtttcgtcgttttgtcgtt

SEQ ID NO: 226 - DNA, 26 bp, Artificial Sequence
  Oligonucleotide
tcgtcgtttgtcgtcggcggccgccg

SEQ ID NO: 227 - DNA, 22 bp, Artificial Sequence
  Oligonucleotide
tcgtcgttttcggcgcgcgccg

SEQ ID NO: 228 - DNA, 22 bp, Artificial Sequence
  Oligonucleotide
tcgtcgttttcggcggccgccg

SEQ ID NO: 229 - DNA, 17 bp, Artificial Sequence
  Oligonucleotide
tcgtcgttttcgtcgtt

SEQ ID NO: 230 - DNA, 22 bp, Artificial Sequence
  Oligonucleotide
tcgtcgttttcggcgcgcgccg

SEQ ID NO: 231 - DNA, 22 bp, Artificial Sequence
  Oligonucleotide
tcgtcgttttcggcggccgccg

SEQ ID NO: 232 - DNA, 16 bp, Artificial Sequence
  Oligonucleotide
tcgtcgttttcgtcgt

SEQ ID NO: 233 - DNA, 17 bp, Artificial Sequence
  Oligonucleotide
tcgtcgttttcgtcgtt

SEQ ID NO: 234 - DNA, 17 bp, Artificial Sequence
  Oligonucleotide
tcgtcgttttcgttgtt

SEQ ID NO: 235 - DNA, 21 bp, Artificial Sequence
  Oligonucleotide
tcgtcgttttgtcgtcgtttt

SEQ ID NO: 236 - DNA, 23 bp, Artificial Sequence
  Oligonucleotide
tcgtcgttttttttcgtcgtttt

SEQ ID NO: 237 - DNA, 17 bp, Artificial Sequence
  Oligonucleotide
tcgtcgtttttgtcgtt

SEQ ID NO: 238 - DNA, 17 bp, Artificial Sequence
  Oligonucleotide
tcgtcgtttttgttgtt

SEQ ID NO: 239 - DNA, 24 bp, Artificial Sequence
  Oligonucleotide
tcgtcgttttntcnttttgtcgtt

SEQ ID NO: 240 - DNA, 16 bp, Artificial Sequence
  Oligonucleotide
tcgtcgttttgacgtt

SEQ ID NO: 241 - DNA, 18 bp, Artificial Sequence
  Oligonucleotide
tcgtcgttttgacgtttt

SEQ ID NO: 242 - DNA, 24 bp, Artificial Sequence
  Oligonucleotide
tcgtcgttttgacgttttgtcgtt

SEQ ID NO: 243 - DNA, 24 bp, Artificial Sequence
  Oligonucleotide
tcgtcgttttgacgttttgtcgtt

SEQ ID NO: 244 - DNA, 16 bp, Artificial Sequence
  Oligonucleotide
tcgtcgttttgtcgtt

SEQ ID NO: 245 - DNA, 24 bp, Artificial Sequence
  Oligonucleotide
tcgtcgttttgtcgttttgtcgtt

SEQ ID NO: 246 - DNA, 24 bp, Artificial Sequence
  Oligonucleotide
tcgtcgttttgtcgttttntcntt

SEQ ID NO: 247 - DNA, 24 bp, Artificial Sequence
  Oligonucleotide
tcgtcgttttgtcgttttgtcgtt

SEQ ID NO: 248 - DNA, 17 bp, Artificial Sequence
  Oligonucleotide
tcgtcgtttngtcgttt

SEQ ID NO: 249 - DNA, 24 bp, Artificial Sequence
  Oligonucleotide
tcgtcgtttngtcgttttgtcgtt

SEQ ID NO: 250 - DNA, 16 bp, Artificial Sequence
  Oligonucleotide
tcgtcgtttgcgtcgt

SEQ ID NO: 251 - DNA, 17 bp, Artificial Sequence
  Oligonucleotide
tcgtcgtttgcgtcgtt

SEQ ID NO: 252 - DNA, 14 bp, Artificial Sequence
  Oligonucleotide
tcgtcgtttgtcgt

SEQ ID NO: 253 - DNA, 15 bp, Artificial Sequence
  Oligonucleotide
tcgtcgtttgtcgtt

SEQ ID NO: 254 - DNA, 24 bp, Artificial Sequence
  Oligonucleotide
tcgtcgnnncgtcgnnnngtcgtt

SEQ ID NO: 255 - DNA, 15 bp, Artificial Sequence
  Oligonucleotide
tcgttttgtcgtttt

SEQ ID NO: 256 - DNA, 19 bp, Artificial Sequence
  Oligonucleotide
tcgttttgtcgtttttttt

SEQ ID NO: 257 - DNA, 17 bp, Artificial Sequence
  Oligonucleotide
tcgttttttttcgtttt

SEQ ID NO: 258 - DNA, 17 bp, Artificial Sequence
  Oligonucleotide
tcgttgttttcgtcgtt

SEQ ID NO: 259 - DNA, 17 bp, Artificial Sequence
  Oligonucleotide
tcgttgttttcgttgtt

SEQ ID NO: 260 - DNA, 17 bp, Artificial Sequence
  Oligonucleotide
tcgttgtttttgtcgtt

SEQ ID NO: 261 - DNA, 17 bp, Artificial Sequence
  Oligonucleotide
tcgttgtttttgttgtt

SEQ ID NO: 262 - DNA, 24 bp, Artificial Sequence
  Oligonucleotide
tcgncgttttgtcgtttngncgtt

SEQ ID NO: 263 - DNA, 25 bp, Artificial Sequence
  Oligonucleotide
tgtcgttgtcgttgtcgttgtcgtt

SEQ ID NO: 264 - DNA, 25 bp, Artificial Sequence
  Oligonucleotide
tgtcgttgtcgttgtcgttgtcgtt

SEQ ID NO: 265 - DNA, 15 bp, Artificial Sequence
  Oligonucleotide
tgtcgtttcgtcgtt

SEQ ID NO: 266 - DNA, 15 bp, Artificial Sequence
  Oligonucleotide
tgtcgttttgtcgtt

SEQ ID NO: 267 - DNA, 20 bp, Artificial Sequence
  Oligonucleotide
ttagttcgtagttcttcgtt

SEQ ID NO: 268 - DNA, 17 bp, Artificial Sequence
  Oligonucleotide
ttcgtcgtttcgtcgtt

SEQ ID NO: 269 - DNA, 18 bp, Artificial Sequence
  Oligonucleotide
ttcgtcgtttcgtcgttt

SEQ ID NO: 270 - DNA, 17 bp, Artificial Sequence
  Oligonucleotide
ttcgtcgttttgtcgtt

SEQ ID NO: 271 - DNA, 20 bp, Artificial Sequence
  Oligonucleotide
ttcgttcttagttcgtagtt

SEQ ID NO: 272 - DNA, 14 bp, Artificial Sequence
  Oligonucleotide
tttcgacgtcgttt

SEQ ID NO: 273 - DNA, 21 bp, Artificial Sequence
  Oligonucleotide
ttttcgtcgttttgtcgtcgt

SEQ ID NO: 274 - DNA, 24 bp, Artificial Sequence
  Oligonucleotide
ttttcgtcgttttgtcgtcgtttt

SEQ ID NO: 275 - DNA, 23 bp, Artificial Sequence
  Oligonucleotide
ttttcgtcgttttttttcgtcgt

SEQ ID NO: 276 - DNA, 26 bp, Artificial Sequence
  Oligonucleotide
ttttcgtcgttttttttcgtcgtttt

SEQ ID NO: 277 - DNA, 24 bp, Artificial Sequence
  Oligonucleotide
ttttcgtcgttttgtcgtcgtttt

SEQ ID NO: 278 - DNA, 15 bp, Artificial Sequence
  Oligonucleotide
ttttcgttttgtcgt

SEQ ID NO: 279 - DNA, 18 bp, Artificial Sequence
  Oligonucleotide
ttttcgttttgtcgtttt

SEQ ID NO: 280 - DNA, 17 bp, Artificial Sequence
  Oligonucleotide
ttttcgttttttttcgt

SEQ ID NO: 281 - DNA, 20 bp, Artificial Sequence
  Oligonucleotide
ttttcgttttttttcgtttt

SEQ ID NO: 282 - DNA, 18 bp, Artificial Sequence
  Oligonucleotide
ttttcgttttgtcgtttt

SEQ ID NO: 283 - DNA, 19 bp, Artificial Sequence
  Oligonucleotide
ttttttttcgttttgtcgt

SEQ ID NO: 284 - DNA, 17 bp, Artificial Sequence
  Oligonucleotide
ttgtcgttttcgtcgtt

SEQ ID NO: 285 - DNA, 17 bp, Artificial Sequence
  Oligonucleotide
ttgtcgttttcgttgtt

SEQ ID NO: 286 - DNA, 17 bp, Artificial Sequence
  Oligonucleotide
ttgtcgtttttgtcgtt

SEQ ID NO: 287 - DNA, 17 bp, Artificial Sequence
  Oligonucleotide
ttgtcgtttttgttgtt

SEQ ID NO: 288 - DNA, 23 bp, Artificial Sequence
  Oligonucleotide
tcgtcgttttgtcgtttgtcgtt

SEQ ID NO: 289 - DNA, 16 bp, Artificial Sequence
  Oligonucleotide
tcgtcgttttgtcgtt

SEQ ID NO: 290 - DNA, 16 bp, Artificial Sequence
  Oligonucleotide
tcgtcgtttcgtcgtt

SEQ ID NO: 291 - DNA, 25 bp, Artificial Sequence
  Oligonucleotide
tgtcgttgtcgttgtcgttgtcgtt

SEQ ID NO: 292 - DNA, 22 bp, Artificial Sequence
  Oligonucleotide
tcgtcgttttcggcggccgccg

SEQ ID NO: 293 - DNA, 24 bp, Artificial Sequence
  Oligonucleotide
tcgtcgttttgtcgttttgtcgtt

SEQ ID NO: 294 - DNA, 24 bp, Artificial Sequence
  Oligonucleotide
tcgtcgttttgtcgttttgtcgtt

SEQ ID NO: 295 - DNA, 24 bp, Artificial Sequence
  Oligonucleotide
tcgtcgttttgtcgttttgtcgtt

SEQ ID NO: 296 - DNA, 24 bp, Artificial Sequence
  Oligonucleotide
tcgtcgttttgtcgttttgtcgtt

SEQ ID NO: 297 - DNA, 24 bp, Artificial Sequence
  Oligonucleotide
tcgtcgttttgtcgttttgtcgtt

SEQ ID NO: 298 - DNA, 24 bp, Artificial Sequence
  Oligonucleotide
tcgtcgttttgtcgttttgtcgtt

SEQ ID NO: 299 - DNA, 24 bp, Artificial Sequence
  Oligonucleotide
tcgtcgttttgtcgttttgtcgtt

SEQ ID NO: 300 - DNA, 24 bp, Artificial Sequence
  Oligonucleotide
tcgtcgttttgtcgttttgtcgtt

SEQ ID NO: 301 - DNA, 24 bp, Artificial Sequence
  Oligonucleotide
tcgtcgttttgtcgttttgtcgtt

SEQ ID NO: 302 - DNA, 24 bp, Artificial Sequence
  Oligonucleotide
tcgtcgttttgtcgttttgtcgtt

SEQ ID NO: 303 - DNA, 24 bp, Artificial Sequence
  Oligonucleotide
tcgtcgttttgtcgttttgtcgtt

SEQ ID NO: 304 - DNA, 24 bp, Artificial Sequence
  Oligonucleotide
tcgtcgttttgtcgttttgtcgtt

SEQ ID NO: 305 - DNA, 24 bp, Artificial Sequence
  Oligonucleotide
tcgtcgttttgtcgttttgtcgtt

SEQ ID NO: 306 - DNA, 24 bp, Artificial Sequence
  Oligonucleotide
tcgtcgttttgtcgttttgtcgtt

SEQ ID NO: 307 - DNA, 24 bp, Artificial Sequence
  Oligonucleotide
tcgtcgttttgtcgttttgtcgtt

SEQ ID NO: 308 - DNA, 24 bp, Artificial Sequence
  Oligonucleotide
tcgtcgttttgtcgttttgtcgtt

SEQ ID NO: 309 - DNA, 24 bp, Artificial Sequence
  Oligonucleotide
tcgtcgttttgtcgttttgtcgtt

SEQ ID NO: 310 - DNA, 24 bp, Artificial Sequence
  Oligonucleotide
tcgtcgttttgtcgttttgtcgtt

SEQ ID NO: 311 - DNA, 24 bp, Artificial Sequence
  Oligonucleotide
tcgtcgttttgtcgttttgtcgtt

SEQ ID NO: 312 - DNA, 24 bp, Artificial Sequence
  Oligonucleotide
tcgtcgttttgtcgttttgtcgtt

SEQ ID NO: 313 - DNA, 24 bp, Artificial Sequence
  Oligonucleotide
tcgtcgttttgtcgttttgtcgtt

SEQ ID NO: 314 - DNA, 24 bp, Artificial Sequence
  Oligonucleotide
tcgtcgttttgtcgttttgtcgtt

SEQ ID NO: 315 - DNA, 24 bp, Artificial Sequence
  Oligonucleotide
tcgtcgttttgtcgttttgtcgtt

SEQ ID NO: 316 - DNA, 24 bp, Artificial Sequence
  Oligonucleotide
tcgtcgttttgtcgttttgtcgtt

SEQ ID NO: 317 - DNA, 24 bp, Artificial Sequence
  Oligonucleotide
tcgtcgttttgtcgttttgtcgtt

SEQ ID NO: 318 - DNA, 24 bp, Artificial Sequence
  Oligonucleotide
tcgtcgttttgtcgttttgtcgtt

SEQ ID NO: 319 - DNA, 24 bp, Artificial Sequence
  Oligonucleotide
tcgtcgttttgtcgttttgtcgtt

SEQ ID NO: 320 - DNA, 24 bp, Artificial Sequence
  Oligonucleotide
tcgtcgttttgtcgttttgtcgtt

SEQ ID NO: 321 - DNA, 24 bp, Artificial Sequence
  Oligonucleotide
tcgtcgttttgtcgttttgtcgtt

SEQ ID NO: 322 - DNA, 24 bp, Artificial Sequence
  Oligonucleotide
tcgtcgttttgtcgttttgtcgtt

SEQ ID NO: 323 - DNA, 24 bp, Artificial Sequence
  Oligonucleotide
tcgtcgttttgtcgttttgtcgtt

SEQ ID NO: 324 - DNA, 24 bp, Artificial Sequence
  Oligonucleotide
tcgtcgttttgtcgttttgtcgtt

SEQ ID NO: 325 - DNA, 24 bp, Artificial Sequence
  Oligonucleotide
tcgtcgttttgtcgttttgtcgtt

SEQ ID NO: 326 - DNA, 24 bp, Artificial Sequence
  Oligonucleotide
tcgtcgttttgtcgttttgtcgtt

SEQ ID NO: 327 - DNA, 24 bp, Artificial Sequence
  Oligonucleotide
tcgtcgttttgtcgttttgtcgtt

SEQ ID NO: 328 - DNA, 24 bp, Artificial Sequence
  Oligonucleotide
tcgtcgttttgtcgttttgtcgtt

SEQ ID NO: 329 - DNA, 24 bp, Artificial Sequence
  Oligonucleotide
tcgtcgttttgtcgttttgtcgtt

SEQ ID NO: 330 - DNA, 24 bp, Artificial Sequence
  Oligonucleotide
tcgtcgttttgtcgttttgtcgtt

SEQ ID NO: 331 - DNA, 24 bp, Artificial Sequence
  Oligonucleotide
tcgtcgttttgtcgttttgtcgtt

SEQ ID NO: 332 - DNA, 24 bp, Artificial Sequence
  Oligonucleotide
tcgtcgttttgtcgttttgtcgtt

SEQ ID NO: 333 - DNA, 24 bp, Artificial Sequence
  Oligonucleotide
tcgtcgttttgtcgttttgtcgtt

SEQ ID NO: 334 - DNA, 24 bp, Artificial Sequence
  Oligonucleotide
tcgtcgttttgtcgttttgtcgtt

SEQ ID NO: 335 - DNA, 24 bp, Artificial Sequence
  Oligonucleotide
tcgtcgttttgtcgttttgtcgtt

SEQ ID NO: 336 - DNA, 24 bp, Artificial Sequence
  Oligonucleotide
tcgtcgttttgtcgttttgtcgtt

SEQ ID NO: 337 - DNA, 24 bp, Artificial Sequence
  Oligonucleotide
tcgtcgttttgtcgttttgtcgtt

SEQ ID NO: 338 - DNA, 24 bp, Artificial Sequence
  Oligonucleotide
tcgtcgttttgtcgttttgtcgtt

SEQ ID NO: 339 - DNA, 24 bp, Artificial Sequence
  Oligonucleotide
tcgtcgttttgtcgttttgtcgtt

SEQ ID NO: 340 - DNA, 24 bp, Artificial Sequence
  Oligonucleotide
tcgtcgttttgtcgttttgtcgtt

SEQ ID NO: 341 - DNA, 24 bp, Artificial Sequence
  Oligonucleotide
tcgtcgttttgtcgttttgtcgtt

SEQ ID NO: 342 - DNA, 24 bp, Artificial Sequence
  Oligonucleotide
tcgtcgttttgtcgttttgtcgtt

SEQ ID NO: 343 - DNA, 24 bp, Artificial Sequence
  Oligonucleotide
tcgtcgttttgtcgttttgtcgtt

SEQ ID NO: 344 - DNA, 24 bp, Artificial Sequence
  Oligonucleotide
tcgtcgttttgtcgttttgtcgtt

SEQ ID NO: 345 - DNA, 24 bp, Artificial Sequence
  Oligonucleotide
tcgtcgttttgtcgttttgtcgtt

SEQ ID NO: 346 - DNA, 24 bp, Artificial Sequence
  Oligonucleotide
tcgtcgttttgtcgttttgtcgtt

SEQ ID NO: 347 - DNA, 24 bp, Artificial Sequence
  Oligonucleotide
tcgtcgttttgtcgttttgtcgtt

SEQ ID NO: 348 - DNA, 24 bp, Artificial Sequence
  Oligonucleotide
tcgtcgttttgtcgttttgtcgtt

SEQ ID NO: 349 - DNA, 24 bp, Artificial Sequence
  Oligonucleotide
tcgtcgttttgtcgttttgtcgtt

SEQ ID NO: 350 - DNA, 24 bp, Artificial Sequence
  Oligonucleotide
tcgtcgttttgtcgttttgtcgtt

SEQ ID NO: 351 - DNA, 24 bp, Artificial Sequence
  Oligonucleotide
tcgtcgttttgtcgttttgtcgtt

SEQ ID NO: 352 - DNA, 24 bp, Artificial Sequence
  Oligonucleotide
tcgtcgttttgtcgttttgtcgtt

SEQ ID NO: 353 - DNA, 24 bp, Artificial Sequence
  Oligonucleotide
tcgtcgttttgtcgttttgtcgtt

SEQ ID NO: 354 - DNA, 24 bp, Artificial Sequence
  Oligonucleotide
tcgtcgttttgtcgttttgtcgtt

SEQ ID NO: 355 - DNA, 24 bp, Artificial Sequence
  Oligonucleotide
tcgtcgttttgtcgttttgtcgtt

SEQ ID NO: 356 - DNA, 24 bp, Artificial Sequence
  Oligonucleotide
tcgtcgttttgtcgttttgtcgtt

SEQ ID NO: 357 - DNA, 24 bp, Artificial Sequence
  Oligonucleotide
tcgtcgttttgtcgttttgtcgtt

SEQ ID NO: 358 - DNA, 24 bp, Artificial Sequence
  Oligonucleotide
tcgtcgttttgtcgttttgtcgtt

SEQ ID NO: 359 - DNA, 24 bp, Artificial Sequence
  Oligonucleotide
tcgtcgttttgtcgttttgtcgtt

SEQ ID NO: 360 - DNA, 24 bp, Artificial Sequence
  Oligonucleotide
tcgtcgttttgtcgttttgtcgtt

SEQ ID NO: 361 - DNA, 24 bp, Artificial Sequence
  Oligonucleotide
tcgtcgttttgtcgttttgtcgtt

SEQ ID NO: 362 - DNA, 24 bp, Artificial Sequence
  Oligonucleotide
tcgtcgttttgtcgttttgtcgtt

SEQ ID NO: 363 - DNA, 24 bp, Artificial Sequence
  Oligonucleotide
tcgtcgttttgtcgttttgtcgtt

SEQ ID NO: 364 - DNA, 24 bp, Artificial Sequence
  Oligonucleotide
tcgtcgttttgtcgttttgtcgtt

SEQ ID NO: 365 - DNA, 24 bp, Artificial Sequence
  Oligonucleotide
tcgtcgttttgtcgttttgtcgtt

SEQ ID NO: 366 - DNA, 24 bp, Artificial Sequence
  Oligonucleotide
tcgtcgttttgtcgttttgtcgtt

SEQ ID NO: 367 - DNA, 24 bp, Artificial Sequence
  Oligonucleotide
tcgtcgttttgtcgttttgtcgtt

SEQ ID NO: 368 - DNA, 24 bp, Artificial Sequence
  Oligonucleotide
tcgtcgttttgtcgttttgtcgtt

SEQ ID NO: 369 - DNA, 24 bp, Artificial Sequence
  Oligonucleotide
tcgtcgttttgtcgttttgtcgtt

SEQ ID NO: 370 - DNA, 24 bp, Artificial Sequence
  Oligonucleotide
tcgtcgttttgtcgttttgtcgtt

SEQ ID NO: 371 - DNA, 24 bp, Artificial Sequence
  Oligonucleotide
tcgtcgttttgtcgttttgtcgtt

SEQ ID NO: 372 - DNA, 24 bp, Artificial Sequence
  Oligonucleotide
tcgtcgttttgtcgttttgtcgtt

SEQ ID NO: 373 - DNA, 24 bp, Artificial Sequence
  Oligonucleotide
tcgtcgttttgtcgttttgtcgtt

SEQ ID NO: 374 - DNA, 24 bp, Artificial Sequence
  Oligonucleotide
tcgtcgttttgtcgttttgtcgtt

SEQ ID NO: 375 - DNA, 24 bp, Artificial Sequence
  Oligonucleotide
tcgtcgttttgtcgttttgtcgtt

SEQ ID NO: 376 - DNA, 24 bp, Artificial Sequence
  Oligonucleotide
tcgtcgttttgtcgttttgtcgtt

SEQ ID NO: 377 - DNA, 24 bp, Artificial Sequence
  Oligonucleotide
tcgtcgttttgtcgttttgtcgtt

SEQ ID NO: 378 - DNA, 24 bp, Artificial Sequence
  Oligonucleotide
tcgtcgttttgtcgttttgtcgtt

SEQ ID NO: 379 - DNA, 24 bp, Artificial Sequence
  Oligonucleotide
tcgtcgttttgtcgttttgtcgtt

SEQ ID NO: 380 - DNA, 24 bp, Artificial Sequence
  Oligonucleotide
tcgtcgttttgtcgttttgtcgtt

SEQ ID NO: 381 - DNA, 24 bp, Artificial Sequence
  Oligonucleotide
tcgtcgttttgtcgttttgtcgtt

SEQ ID NO: 382 - DNA, 24 bp, Artificial Sequence
  Oligonucleotide
tcgtcgttttgtcgttttgtcgtt

SEQ ID NO: 383 - DNA, 24 bp, Artificial Sequence
  Oligonucleotide
tcgtcgttttgtcgttttgtcgtt

SEQ ID NO: 384 - DNA, 24 bp, Artificial Sequence
  Oligonucleotide
tcgtcgttttgtcgttttgtcgtt

SEQ ID NO: 385 - DNA, 24 bp, Artificial Sequence
  Oligonucleotide
tcgtcgttttgtcgttttgtcgtt

SEQ ID NO: 386 - DNA, 24 bp, Artificial Sequence
  Oligonucleotide
tcgtcgttttgtcgttttgtcgtt

SEQ ID NO: 387 - DNA, 24 bp, Artificial Sequence
  Oligonucleotide
tcgtcgttttgtcgttttgtcgtt

SEQ ID NO: 388 - DNA, 24 bp, Artificial Sequence
  Oligonucleotide
tcgtcgttttgtcgttttgtcgtt

SEQ ID NO: 389 - DNA, 24 bp, Artificial Sequence
  Oligonucleotide
tcgtcgttttgtcgttttgtcgtt

SEQ ID NO: 390 - DNA, 24 bp, Artificial Sequence
  Oligonucleotide
tcgtcgttttgtcgttttgtcgtt

SEQ ID NO: 391 - DNA, 24 bp, Artificial Sequence
  Oligonucleotide
tcgtcgttttgtcgttttgtcgtt

SEQ ID NO: 392 - DNA, 24 bp, Artificial Sequence
  Oligonucleotide
tcgtcgttttgtcgttttgtcgtt

SEQ ID NO: 393 - DNA, 24 bp, Artificial Sequence
  Oligonucleotide
tcgtcgttttgtcgttttgtcgtt

SEQ ID NO: 394 - DNA, 24 bp, Artificial Sequence
  Oligonucleotide
tcgtcgttttgtcgttttgtcgtt

SEQ ID NO: 395 - DNA, 24 bp, Artificial Sequence
  Oligonucleotide
tcgtcgttttgtcgttttgtcgtt

SEQ ID NO: 396 - DNA, 24 bp, Artificial Sequence
  Oligonucleotide
tcgtcgttttgtcgttttgtcgtt

SEQ ID NO: 397 - DNA, 24 bp, Artificial Sequence
  Oligonucleotide
tcgtcgttttgtcgttttgtcgtt

SEQ ID NO: 398 - DNA, 24 bp, Artificial Sequence
  Oligonucleotide
tcgtcgttttgtcgttttgtcgtt

SEQ ID NO: 399 - DNA, 24 bp, Artificial Sequence
  Oligonucleotide
tcgtcgttttgtcgttttgtcgtt

SEQ ID NO: 400 - DNA, 24 bp, Artificial Sequence
  Oligonucleotide
tcgtcgttttgtcgttttgtcgtt

SEQ ID NO: 401 - DNA, 24 bp, Artificial Sequence
  Oligonucleotide
tcgtcgttttgtcgttttgtcgtt

SEQ ID NO: 402 - DNA, 24 bp, Artificial Sequence
  Oligonucleotide
tcgtcgttttgtcgttttgtcgtt

SEQ ID NO: 403 - DNA, 24 bp, Artificial Sequence
  Oligonucleotide
tcgtcgttttgtcgttttgtcgtt

SEQ ID NO: 404 - DNA, 24 bp, Artificial Sequence
  Oligonucleotide
tcgtcgttttgtcgttttgtcgtt

SEQ ID NO: 405 - DNA, 24 bp, Artificial Sequence
  Oligonucleotide
tcgtcgttttgtcgttttgtcgtt

SEQ ID NO: 406 - DNA, 24 bp, Artificial Sequence
  Oligonucleotide
tcgtcgttttgtcgttttgtcgtt

SEQ ID NO: 407 - DNA, 24 bp, Artificial Sequence
  Oligonucleotide
tcgtcgttttgtcgttttgtcgtt

SEQ ID NO: 408 - DNA, 24 bp, Artificial Sequence
  Oligonucleotide
tcgtcgttttgtcgttttgtcgtt

SEQ ID NO: 409 - DNA, 24 bp, Artificial Sequence
  Oligonucleotide
tcgtcgttttgtcgttttgtcgtt

SEQ ID NO: 410 - DNA, 24 bp, Artificial Sequence
  Oligonucleotide
tcgtcgttttgtcgttttgtcgtt

SEQ ID NO: 411 - DNA, 24 bp, Artificial Sequence
  Oligonucleotide
tcgtcgttttgtcgttttgtcgtt

SEQ ID NO: 412 - DNA, 24 bp, Artificial Sequence
  Oligonucleotide
tcgtcgttttgtcgttttgtcgtt

SEQ ID NO: 413 - DNA, 24 bp, Artificial Sequence
  Oligonucleotide
tcgtcgttttgtcgttttgtcgtt

SEQ ID NO: 414 - DNA, 24 bp, Artificial Sequence
  Oligonucleotide
tcgtcgttttgtcgttttgtcgtt

SEQ ID NO: 415 - DNA, 24 bp, Artificial Sequence
  Oligonucleotide
tcgtcgttttgtcgttttgtcgtt

SEQ ID NO: 416 - DNA, 24 bp, Artificial Sequence
  Oligonucleotide
tcgtcgttttgtcgttttgtcgtt

SEQ ID NO: 417 - DNA, 24 bp, Artificial Sequence
  Oligonucleotide
tcgtcgttttgtcgttttgtcgtt

SEQ ID NO: 418 - DNA, 24 bp, Artificial Sequence
  Oligonucleotide
tcgtcgttttgtcgttttgtcgtt

SEQ ID NO: 419 - DNA, 24 bp, Artificial Sequence
  Oligonucleotide
tcgtcgttttgtcgttttgtcgtt

SEQ ID NO: 420 - DNA, 24 bp, Artificial Sequence
  Oligonucleotide
tcgtcgttttgtcgttttgtcgtt

SEQ ID NO: 421 - DNA, 24 bp, Artificial Sequence
  Oligonucleotide
tcgtcgttttgtcgttttgtcgtt

SEQ ID NO: 422 - DNA, 24 bp, Artificial Sequence
  Oligonucleotide
tcgtcgttttgtcgttttgtcgtt

SEQ ID NO: 423 - DNA, 24 bp, Artificial Sequence
  Oligonucleotide
tcgtcgttttgtcgttttgtcgtt

SEQ ID NO: 424 - DNA, 24 bp, Artificial Sequence
  Oligonucleotide
tcgtcgttttgtcgttttgtcgtt

SEQ ID NO: 425 - DNA, 24 bp, Artificial Sequence
  Oligonucleotide
tcgtcgttttgtcgttttgtcgtt

SEQ ID NO: 426 - DNA, 24 bp, Artificial Sequence
  Oligonucleotide
tcgtcgttttgtcgttttgtcgtt

SEQ ID NO: 427 - DNA, 24 bp, Artificial Sequence
  Oligonucleotide
tcgtcgttttgtcgttttgtcgtt

SEQ ID NO: 428 - DNA, 24 bp, Artificial Sequence
  Oligonucleotide
tcgtcgttttgtcgttttgtcgtt

SEQ ID NO: 429 - DNA, 10 bp, Artificial Sequence
  Oligonucleotide
nngtcgttnn

SEQ ID NO: 430 - DNA, 16 bp, Artificial Sequence
  Oligonucleotide
nngtcgttgtcgttnn

SEQ ID NO: 431 - DNA, 28 bp, Artificial Sequence
  Oligonucleotide
nngtcgttgtcgttgtcgttgtcgttnn

SEQ ID NO: 432 - DNA, 34 bp, Artificial Sequence
  Oligonucleotide
nngtcgttgtcgttgtcgttgtcgttgtcgttnn

SEQ ID NO: 433 - DNA, 40 bp, Artificial Sequence
  Oligonucleotide
nngtcgttgtcgttgtcgttgtcgttgtcgttgtcgttnn

SEQ ID NO: 434 - DNA, 11 bp, Artificial Sequence
  Oligonucleotide
gggngggnggg

(19) United States
(12) Patent Application Publication
(10) Pub. No.: US 2009/0155307 A1
(43) Pub. Date: Jun. 18, 2009

(54) IMMUNOSTIMULATORY NUCLEIC ACID OIL-IN-WATER FORMULATIONS AND RELATED METHODS OF USE

(75) Inventors: Heather L. Davis, Dunrobin (CA); Michael J. McCluskie, Ottawa (CA)

Correspondence Address: WOLF GREENFIELD & SACKS, P.C., 600 ATLANTIC AVENUE, BOSTON, MA, 02210-2206, US

(73) Assignee: Coley Pharmaceutical Group, Ltd., Ottawa (CA)

(21) Appl. No.: 12/196,201

(22) Filed: Aug. 21, 2008

Related U.S. Application Data
(63) Continuation of application No. 10/816,220, filed on Apr. 1, 2004, now abandoned.
(60) Provisional application No. 60/459,920, filed on Apr. 2, 2003.
(60) Provisional application No. 60/461,903, filed on Apr. 10, 2003.

(51) Int. Cl. A61K 39/12; A61K 39/00; A61K 31/7088

(52) U.S. Cl. 424/204.1; 424/184.1; 514/44

(57) ABSTRACT

The invention involves methods and compositions of an immunostimulatory nucleic acid in oil-in-water emulsions for topical delivery. The compositions can be used to stimulate immune responses, particularly useful in the prevention and/or treatment of infectious disease and cancer.

### RELATED APPLICATIONS

This application claims priority to U.S. Provisional Patent Applications filed Apr. 2, 2003 and Apr. 10, 2003, entitled “IMMUNOSTIMULATORY NUCLEIC ACID OIL-IN-WATER FORMULATIONS AND RELATED METHODS OF USE”, Ser. Nos. 60/459,920 and 60/461,903, respectively, the contents of both of which are incorporated by reference herein in their entirety.

### FIELD OF THE INVENTION

The present invention relates to the use of immunostimulatory nucleic acids in oil-in-water formulation for topical delivery.

### BACKGROUND OF THE INVENTION

In United States alone the death rate due to infectious disease rose 58% between 1980 and 1992. During this time, the use of anti-infective therapies to combat infectious disease has grown significantly and is now a multi-billion dollar a year industry. Even with these increases in anti-infective agent use, the treatment and prevention of infectious disease remains a challenge to the medical community throughout the world. In general, there are three types of anti-infective agents, namely anti-bacterial agents, anti-viral agents, and anti-fungal agents. Within these classes of agents there is some overlap with respect to the type of microorganism they are useful for treating.

One of the problems with anti-infective therapies is the side effects occurring in the host that is treated with the anti-infective agent. For instance, many anti-infectious agents can kill or inhibit a broad spectrum of microorganisms and are not specific for a particular type of species. Treatment with these types of anti-infectious agents results in the killing of the normal microbial flora living in the host, as well as the infectious microorganism. The loss of the microbial flora can lead to disease complications and predispose the host to infection by other pathogens, since the microbial flora compete with and function as barriers to infectious pathogens. Other side effects may arise as a result of specific or non-specific effects of these chemical entities on non-microbial cells or tissues of the host. In the case of antivirals, some of these agents generally are developed specifically for a particular virus, and they are typically only effective while the subject is being medicated with the agent with the chronic viral infection returning as soon as the medication stops. Almost all anti-microbial agents are generally administered systemically even if only a small region of the body is in need of treatment.

In addition to anti-infective agents, vaccines are used to prevent and treat infectious disease. Vaccines include an antigen in combination with an adjuvant. Adjuvants play an important role in the efficacy of vaccines of the treatment and prevention of infectious disease. In addition to increasing the strength and kinetics of an immune response, adjuvants also play a role in determining the type of immune response generated. Aluminum compounds, including aluminum hydroxide and aluminum phosphate, are widely used with human vaccines. These adjuvants skew the immune response towards a T-helper type 2 (Th2) response, which is characterized by the secretion of Th2 type cytokines such as IL-4 and IL-5 and the generation of IgG1 and IgE type antibodies, but weak or absent cytotoxic T lymphocyte (CTL) responses. Development of the appropriate type of immune response is essential for successful immunization. Strong innate immunity, which is associated with a Th1 type immune response, is thought to be essential for the control of intracellular pathogens, whereas strong humoral immunity, which can be found with both Th1 and Th2 type immune responses, appears to be essential for the control of extracellular pathogens. Synthetic oligodeoxynucleotides containing unmethylated CpG dinucleotides (CpG ODN) are novel adjuvants known to promote Th1 type immune responses with the secretion of IFN-γ, TNF-α and IL-12 cytokines, opsonizing antibodies such as those of the IgG2a isotype, and strong CTL induction.

### SUMMARY OF THE INVENTION

The invention provides improved methods and products for the treatment of subjects using immunostimulatory nucleic acids presented in particular formulations. The invention is based, in part, on the finding that when some types of immunostimulatory nucleic acid molecules are particularly formulated, some unexpected and improved results are observed. For instance, the efficacy of the immunostimulatory nucleic acids is profoundly improved when it is formulated in a particular manner as compared to when it is formulated in other manners over the use of the immunostimulatory nucleic acid alone. The results are surprising, in part, because it was previously thought that these different formulations had no effect on the efficacy of the immunostimulatory nucleic acids.

Accordingly, the invention relates in a broad sense to the formulation of immunostimulatory nucleic acids in oil-in-water emulsions (such as for example to a cream consistency), and more particularly as used for topical delivery. Methods and compositions relating to these formulations are provided.

In one aspect, the invention provides a method for inducing an immune response by topically administering to a subject an oil-in-water emulsion and an immunostimulatory nucleic acid in an effective amount to induce an immune response. The immune response induced may involve cells of the innate immune system, which exert early anti-infective effects. The immune response can also involve the adaptive immune system if one or more antigens is present either by active immunization or by virtue of an ongoing or chronic infection. In these latter cases, long lasting antigen-specific responses will be induced. As will be discussed in greater detail herein, the oil-in-water emulsions encompass a variety of emulsions having a range of 1% to 35% oil (or lipid), more preferably 5% to 30%, even more preferably 10% to 25%, and even more preferably 10% to 20%. In some embodiments, the oil in water emulsion is 15% oil. In embodiments involving non-human subjects, one suitable oil-in-water emulsion is EMULSIGEN™.

Thus, in one aspect, the invention provides a method for inducing an antigen-specific immune response by topically administering to a subject an oil-in-water emulsion, an immunostimulatory nucleic acid, and an antigen in an effective amount to induce an antigen-specific immune response. The antigen may be administered at the same site or a different site than the nucleic acid. In embodiments involving non-human subjects, one suitable oil-in-water emulsion is EMULSIGEN™.

The methods of the invention involve the use of an immunostimulatory nucleic acid. The immunostimulatory nucleic acid may be a CpG oligonucleotide and in some embodiments is (TCG TCG TTT TGT CGT TTT GTC GTT; SEQ ID NO:147); (TCG TCG TTT CGT CGT TTC GTC GTT; SEQ ID NO:148) (TCG TCG TTT TTC GGT CGT TTT; SEQ ID NO:149); (TCG TCG TTT CGT CGT TTT GTC GTT; SEQ ID NO:150); (TCG TCG TTT TGT CGT TTT TTT CGA; SEQ ID NO:151); (TCG TCG TTT TTC GTG CGT TTT T; SEQ ID NO:152); (TCGTCGTTGTCGTTTTGTCGTT; SEQ ID NO:153); (TCGCGTGCGTTTTGTCGTTTTGACGTT; SEQ ID NO:154); (TCG TCG TTT GTC GTT TTG TCG TT; SEQ ID NO:155); and/or (GGGGGACGATCGTCGGGGGG; SEQ ID NO:156). Additional immunostimulatory nucleic acids that can be used in the invention include A class, C class and semi-soft immunostimulatory nucleic acids. These are described in greater detail herein and in U.S. Provisional application Ser. No. 10/161,229 filed on Jun. 3, 2002; and U.S. Ser. No. 10/224,523 filed on Aug. 19, 2002, and U.S. 60/404,820 filed on Aug. 19, 2002, the contents of which are incorporated herein in their entirety. The immunuostimulatory nucleic acid may be a T-rich nucleic acid, such as the ODN of SEQ ID NO: 52-57 and/or SEQ ID NO: 62-94 or a poly-G nucleic acid such as the ODN of SEQ ID NO: 46, SEQ ID NO: 47, SEQ ID NO: 58, SEQ ID NO: 61, and/or SEQ ID NO: 95-133. In other embodiments the immunostimulatory nucleic acid may have a sequence selected from the group consisting of SEQ ID NO: 1 through to SEQ ID NO: 146.

The immunostimulatory nucleic acid, such as the CpG immunostimulatory nucleic acid, may be administered a single time or multiple times. If the CpG immunostimulatory nucleic acid is administered multiple times it may be administered at regular intervals, such as, for example, on a daily basis, several times a day, weekly, or monthly basis.

The immunostimulatory nucleic acid, such as the CpG immunostimulatory nucleic acid, is administered topically. The immunostimulatory nucleic acid may be administered to the skin or to the mucosa. Mucosal administration include oral, ocular, nasal, vaginal, rectal and the like.

In some embodiments, the subject has a cancer or an infectious disease or an atopic condition that affects a skin or mucosal surface. In other embodiments, the subject is at risk of developing a cancer or an infectious disease or an atopic condition that affects a skin or mucosal surface. The cancer may be selected from the group consisting of connective tissue cancer, esophageal cancer, eye cancer, larynx cancer, oral cavity cancer, skin cancer, cervical cancer, ovarian cancer, and testicular cancer. The subject may also be an immunocompromised subject. In other embodiments the subject has an infectious disease selected from the group consisting of a viral, bacterial, fungal and parasitic infection. In yet another embodiment, the subject is at risk of developing an infectious diseases elected from the group consisting of a viral, bacterial, fungal and parasitic infection. In important embodiments, the cancer is basal cell carcinoma, melanoma or cervical cancer. In other important embodiments, the infectious disease is a viral infection such as human papilloma viral infection or Herpes simplex viral infection or Herpes zoster viral infection, or a bacterial infection such as superficial infection (e.g., Staphylococcal infection or E. coli infection), or a surface (or topical) parasite infection, or a fungal infection. Preferably the condition is one that exists or implicates topical (skin or mucosal) surfaces. Other conditions to be treated include contact dermatitis, eczema, psoriasis, and other allergic and non-allergic based conditions of topical (skin or mucosal) surfaces. Examples of IgE-associated allergic diseases in humans include anaphylaxis, allergic rhinitis (hayfever), allergic asthma, and atopic dermatitis. Examples of non-allergic inflammation include psoriasis, inflammatory bowel disease (IBD, including Crohn's disease and ulcerative colitis), eczema, allergic contact dermatitis, latex dermatitis, and many types of autoimmune disease.

The immunostimulatory nucleic acid may have a modified backbone, such as a phosphate modified backbone or a peptide modified oligonucleotide backbone. In one embodiment the phosphate modified backbone is a phosphorothioate modified backbone.

In other aspects, the invention provides a composition of an immunostimulatory nucleic acid and an oil-in-water emulsion. In embodiments for non-human subjects, the oil-in-water emulsion is EMULSIGEN™.

In certain embodiments of all aspects of the invention, the immunostimulatory nucleic acid may be a nucleic acid which stimulates a Th1 immune response. Similarly, in some aspects of the invention, it is conceivable that one or more different immunostimulatory nucleic acids may be administered to a subject. Thus depending on the embodiment, one, two, three, four, five or more different immunostimulatory nucleic acids may be administered to a subject in a particular method. Thus, the term “an immunostimulatory nucleic acid” is meant to embrace a single immunostimulatory nucleic acid, a plurality of immunostimulatory nucleic acids of a particular class, and a plurality of immunostimulatory nucleic acids of different classes.

The emulsion and nucleic acid composition may be administered with or without an antigen or with or without an anti-microbial agent. As used herein, an anti-microbial agent refers to agents other than the immunostimulatory nucleic acids of the invention. Accordingly, such anti-microbial agents may be referred to as non-nucleic acid anti-microbial agents, intending that they are distinct from the immunostimulatory nucleic acids of the invention. In some embodiments, the anti-microbial agents are administered in routes independent of the route of administration of the immunostimulatory nucleic acids. The anti-microbial agent may be an anti-bacterial agent, an anti-viral agent, and anti-fungal agent or an anti-parasitic agent. In some embodiments the anti-viral agent is selected from the group consisting of Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

According to other embodiments, the immunostimulatory nucleic acid is administered concurrently with, prior to, or following the administration of other therapeutic agents, e.g., antigen, anti-microbial agents, etc.

In some embodiments, the immunostimulatory nucleic acid is administered in an effective amount for upregulating, enhancing or activating an innate or adaptive (antigen-specific) immune response. In some embodiments, the immunostimulatory nucleic acid is administered in an effective amount for redirecting a pre-existing immune response from a Th2 to a Th1 immune response.

In one aspect the invention relates to a method for reducing viral shedding in a subject by administering to subject infected with a virus or at risk of viral infection, an immunostimulatory nucleic acid and an oil-in-water emulsion in an effective amount to reduce viral shedding. In embodiments involving non-human animals, the oil-in-water emulsion is EMULSIGEN™. The non-human animal may be a dog, cat, horse, cow, pig, sheep, goat, primate or chicken. If the subject is a human subject, the emulsion may be any of those taught herein including those having 1%, 5%, 10%, 15%, 20%, 25%, 30%, or 35% oil compositions. As used herein, an “oil” percentage intends the total amount of lipid or lipid soluble components in the emulsion.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIG. 1 is a graph showing the effect of nucleic acid (100 μg) administered via water-in-oil cream or saline formulations on mean pathology scores and percent survival.

FIG. 2 is a graph showing the effect of nucleic acid (100 μg) administered via oil-in-water cream or saline formulations on mean pathology scores and percent survival.

FIG. 3 is a graph showing the effect of nucleic acid (10 μg) administered via oil-in-water cream or saline formulations on mean pathology scores and percent survival.

It is to be understood that the figures are not required to enable the invention.

### DETAILED DESCRIPTION OF THE INVENTION

It was surprisingly discovered according to the invention that select combinations of immunostimulatory nucleic acids and therapeutic formulations such as oil-in-water emulsions work dramatically better, and sometimes even synergistically, to improve an immune response than other nucleic acid for mutations, particularly when used topically. Although many formulations have been developed and tested for administering drugs, these particular types dramatically enhance the activity of the immunostimulatory nucleic acids. This was surprising, in part, because other similar formulations did not demonstrate the same dramatic types of improvements as the therapeutic formulations described herein. The term “therapeutic formulations” as used herein refers to oil-in-water emulsions. An example of an oil-in-water emulsion is such as EMULSIGEN™ which is used in non-human subjects.

The oil-in-water emulsions of the invention that are useful for administration to humans include oil or lipid constituents such as white petrolatum, white wax, caprylic/capric triglyceride, stearyl alcohol, and the like. Other oil or lipid constituents can be added or substituted into the formulations. The emulsions further contain water soluble constituents, surfactants such as steareth 21 or 2 or sorbitan monooleate, thickeners such as carbopol 981, and/or preservatives such as methylparaben and propylparaben.

The oil or lipid to water ratio in the formulation may vary from below 1% oil to over 35% oil (and every percentage therebetween). The higher the oil content, however, the greater the dependency on surfactant in order to emulsify as much of the oil as possible. In some embodiments, the oil constituents comprise 1%, 5%, 10%, 15%, 20%, 25%, 30%, 35%, or more of the formulation (w/w). In some important embodiments, the oil constituents comprise between 1% and 35%, or between 5% and 25%, or between 10% and 20%. In an important embodiment, the oil constituents represent 15% (w/w) of the formulation. Such a formulation also preferably comprises less than 5% surfactant, less than 4% surfactant or less than 3% surfactant.

As demonstrated in the Examples described below the combination of immunostimulatory nucleic acids and oil-in-water emulsions have demonstrated significantly improved therapeutic effects in the treatment and prevention of infectious disease when administered topically. Accordingly, in preferred embodiments, the oil-in water and immunostimulatory nucleic acid combinations are administered topically (e.g., to a skin or mucosal surface). When administered to a mucosal surface, it is preferred that the emulsions be administered to an external mucosal surface, such as the vagina, oral cavity, nasal cavity and the like.

The combination of immunostimulatory nucleic acids with oil-in-water emulsion when delivered topically (e.g., to the skin or mucosa) can be used to reduce viral shedding. This is an extremely important because it reflects the degree of control over the infection and the level to which the infected subject could be contagious to others. “Viral shedding” refers to production of viral particles at a mucosal surface by an animal infected with a virus. The presence or absence of viral shedding can be determined by taking a sample from an animal (i.e., nasal or vaginal secretions) and analyzing the sample for the presence of virus. If a drug prevents viral shedding it means that it is effectively controlling the rate of viral replication and that it effectively prevents transmission of the infection to another subject, as well as spread of the infection within the infected subject. The ability of the nucleic acids in the therapeutic formulations of the invention to reduce and even eliminate viral shedding demonstrates the surprising potency of the composition.

The immunostimulatory nucleic acids are useful for treating or preventing infectious disease in a subject. A “subject” shall mean a human or vertebrate mammal including, but not limited to, a dog, cat, horse, cow, pig, sheep, goat, or primate, e.g., monkey. In some embodiments a subject specifically excludes rodents such as mice.

Thus the immunostimulatory nucleic acids combined with the therapeutic formulations stimulate the immune system to prevent or treat infectious disease. The strong yet balanced, cellular and humoral immune responses that result from the immune stimulatory capacity of the nucleic acid reflect the natural defense system of the subject against invading microorganisms.

As used herein, the term “prevent”, “prevented”, or “preventing” and “treat”, “treated” or “treating” when used with respect to the prevention of an infectious disease refers to a prophylactic treatment which increases the resistance of a subject to a microorganism or, in other words, decreases the likelihood that the subject will develop an infectious disease to the microorganism. Furthermore, as used herein, the term treat”, “treated” or “treating” when used with respect to the treatment of an infectious disease refers to a post-exposure treatment which increases the ability of a subject to fight an infection by a microorganism or, in other words, increases the ability of the subject to fight and overcome a pre-existing infection by the microorganism, e.g., reduce or eliminate it altogether or prevent it from becoming worse.

The invention provides methods for inducing immune responses, and more preferably local immune responses. Local immune responses can be induced by the localized delivery of an immunostimulatory nucleic acid, such as those taught herein. Depending upon the topical site to which the emulsion is administered, the ensuing immune response may also be systemic in nature. In preferred embodiments, however, where the disease or condition is localized, a local immune response is preferred.

The immunostimulatory nucleic acids are useful in some aspects of the invention as a prophylactic therapy of a subject at risk of developing an infectious disease where the exposure of the subject to a microorganism or expected exposure to a microorganism is known or suspected. A “subject at risk” of developing an infectious disease as used herein is a subject who has any risk of exposure to a microorganism, e.g. someone who is in contact with an infected subject or who is traveling to a place where a particular microorganism is found. For instance, a subject at risk may be a subject who is planning to travel to an area where a particular microorganism is found or it may even be any subject living in an area where a microorganism has been identified. A subject at risk of developing an infectious disease includes those subjects that have a general risk of exposure to a microorganism, e.g., influenza, but that don't have the active disease during the treatment of the invention as well as subjects that are considered to be at specific risk of developing an infectious disease because of medical or environmental factors, that expose them to a particular microorganism.

A “subject having an infectious disease” is a subject that has had contact with a microorganism and the microorganism has invaded the body of the subject, potentially replicating in the subject in the process. The word “invade” as used herein refers to contact by the microorganism with the external surface of the subject, e.g., skin or mucosal membranes and/or refers to the penetration of the external surface of the subject by the microorganism. External surfaces that are open (for example via a wound or lesion) are more susceptible to penetration by microorganisms.

An “infectious disease” as used herein, refers to a disorder arising from the invasion of a host, superficially, locally, or systemically, by an infectious microorganism. Infectious microorganisms include bacteria, viruses, fungi and parasites.

Bacteria are unicellular organisms that multiply asexually by binary fission. They are classified and named based on their morphology, staining reactions, nutrition and metabolic requirements, antigenic structure, chemical composition, and genetic homology. Bacteria can be classified into three groups based on their morphological forms, spherical (coccus), straight-rod (bacillus) and curved or spiral rod (vibrio, campylobacter, spirillum, and spirochaete). Bacteria are also more commonly characterized based on their staining reactions into two classes of organisms, gram-positive and gram-negative. Gram refers to the method of staining which is commonly performed in microbiology labs. Gram-positive organisms retain the stain following the staining procedure and appear a deep violet color. Gram-negative organisms do not retain the stain but take up the counter-stain and thus appear pink.

The invention intends to encompass the prevention or treatment of bacterial infections that are most likely to infect a wound on an external surface of a subject such as the dermal or mucosal external surfaces.

Infectious bacteria include, but are not limited to, gram negative and grain positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Viruses are small infectious agents that contain a nucleic acid core and a protein coat, but are not independently living organisms. A virus cannot survive in the absence of a living cell within which it can replicate. Viruses enter specific living cells either by endocytosis or direct injection of DNA (phage) and multiply, causing disease. The multiplied virus can then be released and infect additional cells. Some viruses are DNA-containing viruses and other are RNA-containing viruses.

Once the virus enters the cell it uses the cell's metabolic machinery to produce new viral proteins that assemble into new infectious units. This process of viral replication can cause a variety of physiological effects in the infected cell. One effect is cell degeneration, in which the accumulation of virus within the cell causes the cell to die and break into pieces and release the virus. Another effect is that the infected cell is not destroyed but the newly produced virus is able to escape by other means, after which it can infect neighboring cells or it can enter the circulation and reach other areas of the body and infect distant cells. Yet another effect is cell fusion, in which infected cells fuse with neighboring cells to produce syncytia. Other types of virus cause cell proliferation, which can result in tumor formation.

In important embodiments, the invention intends to encompass the prevention and treatment of viral infections such as human papilloma viral infection, Herpes simplex viral infection and Herpes zoster viral infection.

Infectious virus of both human and non-human vertebrates, include RNA viruses and DNA viruses, which means that the genetic material that encodes the viral proteins is RNA or DNA respectively. Viruses can include, but are not limited to, enteroviruses (including, but not limited to, viruses that the family picornaviridae, such as polio virus, coxsackie virus, echo virus), rotaviruses, adenovirus, hepatitis. Specific examples of viruses that have been found in humans include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis A virus, Hepatitis B virus, Hepatitis C virus, Hepatitis E virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), Norwalk and related viruses, and astroviruses).

In addition to viruses that infect human subjects, the invention is also useful for treating viruses that infect non-human vertebrates. For instance, in addition to the prevention and treatment of infectious human diseases, the methods of the invention are also useful in prevention and treatment of infectious disease in non-human subjects.

Retroviruses that infect non-human vertebrates include both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are infectious in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that infect vertebrate animals include, but are not limited to the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as amatatoxin and phallotoxin produced by poisonous mushrooms and aflotoxins, produced by aspergillus species, not all fungi cause infectious disease.

Most fungi are able to infect external surfaces such as the skin and external mucosa (i.e., superficial infections). Accordingly, the invention embraces the prevention and treatment of fungal infections that occur at external surfaces, as described herein, in some important embodiments.

Infectious fungi can cause systemic or superficial infections. Primary systemic infection can occur in normal healthy subjects and opportunistic infections, are most frequently found in immunocompromised subjects. The most common fungal agents causing primary systemic infection include blastomyces, coccidioides, and histoplasma. Common fungi causing opportunistic infection in immuno-compromised or immunosuppressed subjects include, but are not limited to, candida albicans (an organism which is normally part of the respiratory tract flora), cryptococcus neoformans (sometimes in normal flora of respiratory tract), and various aspergillus species. Systemic fungal infections are invasive infections of the internal organs. The organism usually enters the body through the lungs, gastrointestinal tract, or intravenous lines. These types of infections can be caused by primary pathogenic fungi or opportunistic fungi.

Superficial fungal infections involve growth of fungi on an external surface without invasion of internal tissues. Typical superficial fungal infections include cutaneous fungal infections involving skin, hair, or nails. An example of a cutaneous infection is Tinea infections, such as ringworm, caused by dermatophytes, such as microsporum or traicophyton species, i.e., microsporum canis, microsporum gypsum, tricofitin rubrum. Examples of fungi include: Cryptococcus neoformans, Histoplasma capsulatum, Coccidioides immitis, Blastomyces dermatitidis, Chlamydia trachomatis, Candida albicans.

Parasites are non-viral microorganisms which depend upon other organisms in order to survive and thus must enter, or infect, another organism to continue their life cycle. The infected organism, i.e., the host, provides both nutrition and habitat to the parasite. Parasites refer to protozoa, helminths, and ectoparasitic arthropods (e.g., ticks, mites, etc.). Protozoa are single celled organisms which can replicate both intracellularly and extracellularly, particularly in the blood, intestinal tract or the extracellular matrix of tissues. Helminths are multicellular organisms which almost always are extracellular (the exception being Trichinella spp.). Helminths normally require exit from a primary host and transmission into a secondary host in order to replicate. In contrast to these aforementioned classes, ectoparasitic arthropods form a parasitic relationship with the external surface of the host body.

Parasites are capable of infecting almost any tissue or cell type, however, depending on the particular parasite, they tend to preferentially target a subset of cells including, in humans, red cells, fibroblasts, muscle cells, macrophages and hepatocytes. For example, the protozoan Entamoeba histolytica which is found in the intestinal tract and propagated by contact with host feces, can migrate across the intestinal mucosal lining to infect other bodily tissues such as the liver eventually forming amoebic abscesses. Other parasites can be transmitted via intermediate hosts such as mosquitoes. Ectoparasitic arthropods are a nuisance for household pets (e.g., dogs, cats) and, more importantly, can contribute to wasting syndromes and act as a vehicle for the transmission of other infections (such as babesiosis and theileriasis) in agricultural livestock.

Parasites can be classified based on whether they are intracellular or extracellular. An “intracellular parasite” as used herein is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at lest one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp. In one aspect, the invention relates to the prevention and treatment of infection resulting from intracellular parasites and obligate intracellular parasites which have at least in one stage of their life cycle that is intracellular. In some embodiments, the invention is directed to the prevention of infection from obligate intracellular parasites which are predominantly intracellular. The methods of the invention are not expected to function in the prevention of infection by extracellular parasites, i.e., helminths. An exemplary and non-limiting list of parasites for some aspects of the invention is provided herein.

Parasitic infections targeted by the methods of the invention include those caused by the following parasites Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis and Leishmania tropica, Trypanosoma gambiense, Trypanosmoma rhodesiense and Schistosoma mansoni. In preferred embodiments, the method is directed towards the prevention of infection with parasites which cause malaria.

Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative, and is not intended to be limiting.

In some embodiments, the invention is particularly directed to infectious diseases that are incurred by exposure at a topical surface, such as the skin or a mucosal surface: One example of such diseases in sexually transmitted diseases (STD) that are incurred through vaginal, rectal or oral exposure. As used herein, an STD is an infection that is transmitted primarily, but not exclusively, through sexual intercourse. In addition to being transmitted via sexual contact with an infected subject, some STDs can also be transmitted through contact with bodily fluids of an infected subject. As used herein, “a bodily fluid” includes blood, saliva, semen, vaginal fluids, urine, feces and tears. STDs are most commonly transmitted through blood, saliva, semen and vaginal fluids. As an example, blood and blood product transfusions are common modes of transmission for many sexually transmitted pathogens, including HIV and Hepatitis viruses.

STDs intended to be prevented or treated by the methods and compositions of the invention include gonorrhoeae, syphilis, chlamydia, HPV (causing genital warts and cervical dysplasia), AIDS/HIV, hepatitis B, herpes simplex viruses I and II, trichomonas, candida, and chancroid, but are not so limited. Other STDs intended to be prevented or treated by the methods and compositions provided herein are scabies and pubic lice infections.

Sexually transmitted pathogens are generally bacterial, viral, parasitic or fungal in nature. Organisms that cause STDs include bacteria such as Neisseria gonorrhoeae, Chlamydia trachomatis, Treponema pallidum, Haemophilus ducreyi, Condyloma acuminata, Calymmatobacterium granulomatis and Ureaplasma urealyticum, viruses such as Human immunodeficiency viruses (HIV-1 and HIV-2), Human T lymphotropic virus type I (HTLV-I), Herpes simplex virus type 2 (HSV-2), Human papilloma virus (multiple types), Hepatitis B virus, Cytomegalovirus and Molluscum contagiosum virus, parasites such as Trichomonas vaginalis and Phthirus pubis, and fungi such as Candida albicans.

Other infections are known to be sexually transmitted, even if sexual transmission is not their predominant mode of transmission. This latter category includes infections caused by bacteria such as Mycoplasma hominis, Gardnerella vaginalis and Group B streptococcus, viruses such as Human T lymphotrophic virus type II (HTLV-II), hepatitis C and D viruses, Herpes simplex virus type I (HSV-1) and Epstein-Barr virus (EBV), and parasites such as Sarcoptes scabiei.

The invention also intends to embrace STDs or other infections that are transmitted by sexual contact involving oral-fecal exposure. These infections are caused by bacteria such as Shigella spp. and Campylobacter spp., viruses such as hepatitis A virus and parasites such as Giardia lamblia and Entamoeba histolytica.

In another aspect, the invention is intended to prevent or treat STD-related conditions. STD-related conditions are conditions, disorders or diseases which result from an STD (i.e., they are secondary to the initial sexually transmitted infection). These include acute arthritis (N. gonorrhoeae (e.g., DGI), C. trachomatis (e.g., Reiter's syndrome), HBV, HIV), acute pelvic inflammatory disease (N. gonorrhoeae, C. trachomatis, BV-associated bacteria), AIDS (HIV-1, HIV-2; HSV, also many opportunistic pathogens), bacterial vaginosis (BV) (BV-associated bacteria), cervicitis (C. trachomatis), cystitis/urethritis (C. trachomatis, N. gonorrhoeae, HSV), enteritis, enterocolitis, epididymitis (C. trachomatis, N. gonorrhoeae), epididymo-orchitis (inflammation of the epididymis and testes) (N. gonorrhoeae), genital and anal warts (Human papillomavirus (genital types), gonococcal dermititis, hepatocellular carcinoma (HBV), Kaposi's sarcoma (HIV), lower genital tract infections: females mucopurulent cervicitis (C. trachomatis, N. gonorrhoeae), lymphoid neoplasia (HIV, HTLV-I), mononucleosis syndrome (Cytomegalovirus, HIV EBV), neoplasias, pharyngitis (N. gonorrhoeae), proctitis (C. trachomatis, N. gonorrhoeae, HSV, T. pallidum), proctocolitis (G. lamblia, Campylobacter spp., Shigella spp., E. histolytica, other enteric pathogens), prostatitis (prostate inflammation) (N. gonorrhoeae), public lice (P. pubis), Reiter's syndrome, salpingitis, scabies (S. scabiei), septicemia, squamous cell cancer of the cervis, anus, vulva, or penis (Human papillomavirus (especially types 16, 18, 31), tropical spastic paraparesis (HTLV-1), ulcerative lesions of the genitalia (HSV-1, T. pallidum, H. ducreyi, C. trachomatis (LGV strains), C. granulomatis), urethritis in males (N. gonorrhoeae, C. trachomatis, U. urealyticum, USV), urethritis in females (C. trachomatis), vaginitis (C. trachomatis), viral hepatitis (HBV), and vulvovaginitis (C. albicans, T. vaginalis). The existence of some forms of STD, for example, trichomonas, in a female subject sometimes result in an imbalance in the endogenous bacteria of the vagina and as a result yeast infections are quite common. Thus, by preventing or treating STDs such as trichomonas, the invention also provides a method for preventing or treating an STD-related yeast infection.

The combination of emulsion/nucleic acid compositions may also be administered in conjunction with an anti-microbial agent for the treatment or prevention of infectious disease. An anti-microbial agent, as used herein, refers to a naturally-occurring or synthetic compound which is capable of directly killing or inhibiting infectious microorganisms. These agents are distinct from the immunostimulatory nucleic acids discussed herein, and thus may be referred to as non-nucleic acid anti-microbial agents. The type of anti-microbial agent useful according to the invention will depend upon the type of microorganism with which the subject is infected or at risk of becoming infected. One type of anti-microbial agent is an anti-bacterial agent. Anti-bacterial agents kill or inhibit the growth or function of bacteria. A large class of anti-bacterial agents is antibiotics.

Anti-viral agents are compounds that prevent infection of cells by viruses or replication of the virus within the cell. There are many fewer anti-viral drugs than anti-bacterial drugs because the process of viral replication is so closely related to DNA replication within the host cell, that non-specific anti-viral agents would often be toxic to the host. Therefore, individual highly specific anti-viral agents need to be developed against individual viruses. There are several stages within the process of viral infection which can be blocked or inhibited by anti-viral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogues), maturation of new virus proteins (e.g. protease inhibitors), and budding and release of the virus.

Anti-fungal agents are useful for the treatment and prevention of infective fungi directly. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream). In some important embodiments, the anti-fungal agent of choice, preferably in the prevention or treatment of Candida albicans infection may be selected from the group of amphoterizin B, miconazole, clotrimazole, 5-fluorocytosine, fluconazole, fluconazole, itraconazole and voriconazole. Other such compounds are known in the art and are generally commercially available.

Parasitides are agents that kill parasites, preferably directly. Examples of parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Parasiticides used in non-human subjects include piperazine, diethylcarbamazine, thiabendazole, fenbendazole, albendazole, oxfendazole, oxibendazole, febantel, levamisole, pyrantel tartrate, pyrantel pamoate, dichlorvos, ivermectin, doramectic, milbemycin oxime, iprinomectin, moxidectin, N-butyl chloride, toluene, hygromycin B thiacetarsemide sodium, melarsomine, praziquantel, epsiprantel, benzimidazoles such as fenbendazole, albendazole, oxfendazole, clorsulon, albendazole, amprolium; decoquinate, lasalocid, monensin sulfadimethoxine; sulfamethazine, sulfaquinoxaline, metronidazole.

Parasiticides used in horses include mebendazole, oxfendazole, febantel, pyrantel, dichlorvos, trichlorfon, ivermectin, piperazine; for S. westeri: ivermectin, benzimiddazoles such as thiabendazole, cambendazole, oxibendazole and fenbendazole. Useful parasiticides in dogs include milbemycin oxine, ivermectin, pyrantel pamoate and the combination of ivermectin and pyrantel. The treatment of parasites in swine can include the use of levamisole, piperazine, pyrantel, thiabendazole, dichlorvos and fenbendazole. In sheep and goats anthelmintic agents include levamisole or ivermectin. Caparsolate has shown some efficacy in the treatment of D. immitis (heartworm) in cats.

Agents used in the prevention and treatment of protozoal diseases in poultry, particularly trichomoniasis, can be administered in the feed or in the drinking water and include protozoacides such as aminonitrothiazole, dimetridazole (Emtryl), nithiazide (Hepzide) and Enheptin. However, some of these drugs are no longer available for use in agrigultural stocks in the USA. Back yard flocks or pigeons not used for food production may be effectively treated with dimetridazole by prescription of a veterinarian (1000 mg/L in drinking water for 5-7 days).

In addition to the use of the emulsion/nucleic acid composition to prevent or treat conditions in humans, the methods provided herein are also suited for prevention and treatment in non-human vertebrates. Non-human vertebrates which exist in close quarters and which are allowed to intermingle as in the case of zoo, farm and research animals are also embraced as subjects for the methods of the invention. Zoo animals such as the felid species including for example lions, tigers, leopards, cheetahs, and cougars; elephants, giraffes, bears, deer, wolves, yaks, non-human primates, seals, dolphins and whales; and research animals such as mice, rats, hamsters and gerbils are all potential subjects for the methods of the invention.

Birds such as hens, chickens, turkeys, ducks, geese, quail, and pheasant are prime targets for many types of infections. Hatching birds are exposed to pathogenic microorganisms shortly after birth. Although these birds are initially protected against pathogens by maternal derived antibodies, this protection is only temporary, and the bird's own immature immune system must begin to protect the bird against the pathogens. It is often desirable to prevent infection in young birds when they are most susceptible. It is also desirable to prevent against infection in older birds, especially when the birds are housed in closed quarters, leading to the rapid spread of disease. Thus, it is desirable to administer the immunostimulatory nucleic acids and anti-microbial agents to birds to prevent infectious disease.

An example of a common infection in chickens is chicken infectious anemia virus (CIAV). CIAV was first isolated in Japan in 1979 during an investigation of a Marek's disease vaccination break (Yuasa et al., 1979, Avian Dis. 23:366-385). Since that time, CIAV has been detected in commercial poultry in all major poultry producing countries (van Bulow et al., 1991, pp. 690-699) in Diseases of Poultry, 9th edition, Iowa State University Press).

CIAV infection results in a clinical disease, characterized by anemia, hemorrhage and immunosuppression, in young susceptible chickens. Atrophy of the thymus and of the bone marrow and consistent lesions of CIAV-infected chickens are also characteristic of CIAV infection. Lymphocyte depletion in the thymus, and occasionally in the bursa of Fabricius, results in immunosuppression and increased susceptibility to secondary viral, bacterial, or fungal infections which then complicate the course of the disease. The immunosuppression may cause aggravated disease after infection with one or more of Marek's disease virus (MDV), infectious bursal disease virus, reticuloendotheliosis virus, adenovirus, or reovirus. It has been reported that pathogenesis of MDV is enhanced by CIAV (DeBoer et al., 1989, p. 28 In Proceedings of the 38th Western Poultry Diseases Conference, Tempe, Ariz.). Further, it has been reported that CIAV aggravates the signs of infectious bursal disease (Rosenberger et al., 1989, Avian Dis. 33:707-713). Chickens develop an age resistance to experimentally induced disease due to CAA. This is essentially complete by the age of 2 weeks, but older birds are still susceptible to infection (Yuasa, N. et al., 1979 supra; Yuasa, N. et al., Arian Diseases 24, 202-209, 1980). However, if chickens are dually infected with CAA and an immunosuppressive agent (IBDV, MDV etc.) age resistance against the disease is delayed (Yuasa, N. et al., 1979 and 1980 supra; Bulow von V. et al., J. Veterinary Medicine 33, 93-116, 1986). Characteristics of CIAV that may potentiate disease transmission include high resistance to environmental inactivation and some common disinfectants. The economic impact of CIAV infection on the poultry industry is clear from the fact that 10% to 30% of infected birds in disease outbreaks die.

Cattle and livestock are also susceptible to infection. Disease which affect these animals can produce severe economic losses, especially amongst cattle. The methods of the invention can be used to protect against infection in livestock, such as cows, horses, pigs, sheep, and goats.

Cows can be infected by bovine viruses. Bovine viral diarrhea virus (BVDV) is a small enveloped positive-stranded RNA virus and is classified, along with hog cholera virus (HOCV) and sheep border disease virus (BDV), in the pestivirus genus. Although, Pestiviruses were previously classified in the Togaviridae family, some studies have suggested their reclassification within the Flaviviridae family along with the flavivirus and hepatitis C virus (HCV) groups (Francki, et al., 1991).

BVDV, which is an important pathogen of cattle can be distinguished, based on cell culture analysis, into cytopathogenic (CP) and noncytopathogenic (NCP) biotypes. The NCP biotype is more widespread although both biotypes can be found in cattle. If a pregnant cow becomes infected with an NCP strain, the cow can give birth to a persistently infected and specifically immunotolerant calf that will spread virus during its lifetime. The persistently infected cattle can succumb to mucosal disease and both biotypes can then be isolated from the animal. Clinical manifestations can include abortion, teratogenesis, and respiratory problems, mucosal disease and mild diarrhea. In addition, severe thrombocytopenia, associated with herd epidemics, that may result in the death of the animal has been described and strains associated with this disease seem more virulent than the classical BVDVs.

Equine herpesviruses (EHV) comprise a group of antigenically distinct biological agents which cause a variety of infections in horses ranging from subclinical to fatal disease. These include Equine herpesvirus-1 (EHV-1), a ubiquitous pathogen in horses. EHV-1 is associated with epidemics of abortion, respiratory tract disease, and central nervous system disorders. Primary infection of upper respiratory tract of young horses results in a febrile illness which lasts for 8 to 10 days. Immunologically experienced mares may be reinfected via the respiratory tract without disease becoming apparent, so that abortion usually occurs without warning. The neurological syndrome is associated with respiratory disease or abortion and can affect animals of either sex at any age, leading to in-coordination, weakness and posterior paralysis (Telford, E. A. R. et al., Virology 189, 304-316, 1992). Other EHV's include EHV-2, or equine cytomegalovirus, EHV-3, equine coital exanthema virus, and EHV-4, previously classified as EHV-1 subtype 2.

Sheep and goats can be infected by a variety of dangerous microorganisms including visna-maedi.

Primates such as monkeys, apes and macaques can be infected by simian immunodeficiency virus. Inactivated cell-virus and cell-free whole simian immunodeficiency vaccines have been reported to afford protection in macaques (Stott et al. (1990) Lancet 36:1538-1541; Desrosiers et al. PNAS USA (1989) 86:6353-6357; Murphey-Corb et al. (1989) Science 246:1293-1297; and Carlson et al. (1990) AIDS Res. Human Retroviruses 6:1239-1246). A recombinant HIV gp120 vaccine has been reported to afford protection in chimpanzees (Berman et al. (1990) Nature 345:622-625).

Cats, both domestic and wild, are susceptible to infection with a variety of microorganisms. For instance, feline infectious peritonitis is a disease which occurs in both domestic and wild cats, such as lions, leopards, cheetahs, and jaguars. When it is desirable to prevent infection with this and other types of pathogenic organisms in cats, the methods of the invention can be used to prevent or treat infection in cats.

Domestic cats may become infected with several retroviruses, including but not limited to feline leukemia virus (FeLV), feline sarcoma virus (FeSV), endogenous type C oncornavirus (RD-114), and feline syncytia-forming virus (FeSFV). Of these, FeLV is the most significant pathogen, causing diverse symptoms, including lymphoreticular and myeloid neoplasms, anemias, immune mediated disorders, and an immunodeficiency syndrome which is similar to human acquired immune deficiency syndrome (AIDS). Recently, a particular replication-defective FeLV mutant, designated FeLV-AIDS, has been more particularly associated with immunosuppressive properties.

The discovery of feline T-lymphotropic lentivirus (also referred to as feline immunodeficiency) was first reported in Pedersen et al. (1987) Science 235:790-793. Characteristics of FIV have been reported in Yamamoto et al. (1988) Leukemia, December Supplement 2:204 S-215S; Yamamoto et al. (1988) Am. J. Vet. Res. 49:1246-1258; and Ackley et al. (1990) J. Virol. 64:5652-5655. Cloning and sequence analysis of FIV have been reported in Olmsted et al. (1989) Proc. Natl. Acad. Sci. USA 86:2448-2452 and 86:4355-4360.

Feline infectious peritonitis (FIP) is a sporadic disease occurring unpredictably in domestic and wild Felidae. While FIP is primarily a disease of domestic cats, it has been diagnosed in lions, mountain lions, leopards, cheetahs, and the jaguar. Smaller wild cats that have been afflicted with FIP include the lynx and caracal, sand cat, and pallas cat. In domestic cats, the disease occurs predominantly in young animals, although cats of all ages are susceptible. A peak incidence occurs between 6 and 12 months of age. A decline in incidence is noted from 5 to 13 years of age, followed by an increased incidence in cats 14 to 15 years old.

Viral, bacterial, and parasitic diseases in fin-fish, shellfish or other aquatic life forms pose a serious problem for the aquaculture industry. Owing to the high density of animals in the hatchery tanks or enclosed marine farming areas, infectious diseases may eradicate a large proportion of the stock in, for example, a fin-fish, shellfish, or other aquatic life forms facility. The fish immune system has many features similar to the mammalian immune system, such as the presence of B cells, T cells, lymphokines, complement, and immunoglobulins. Fish have lymphocyte subclasses with roles that appear similar in many respects to those of the B and T cells of mammals.

Aquaculture species include but are not limited to fin-fish, shellfish, and other aquatic animals. Fin-fish include all vertebrate fish, which may be bony or cartilaginous fish, such as, for example, salmonids, carp, catfish, yellowtail, seabream, and seabass. Salmonids are a family of fin-fish which include trout (including rainbow trout), salmon, and Arctic char. Examples of shellfish include, but are not limited to, clams, lobster, shrimp, crab, and oysters. Other cultured aquatic animals include, but are not limited to eels, squid, and octopi.

In addition to the human health risks, parasites also pose a considerable risk to agricultural livestock and domestic and wild animals. Agricultural livestock and in some cases zoo animals are ripe targets for widespread transmission of parasitic diseases for two major reasons. First, livestock usually live in such close quarters thereby facilitating the transmission of a parasite to an entire flock or herd. Second, because many enteric parasites eventually exit the body in feces which invariably litter a grazing field for animals, the likelihood of transmission and widespread infection is high. Thus the maintenance of a parasite free environment through prevention of parasitic infections would be highly desirable in these circumstances.

Typical parasites infecting horses are Gasterophilus spp.; Eimeria leuckarti, Giardia spp.; Tritrichomonas equi; Babesia spp. (RBC's), Theileria equi; Trypanosoma spp.; Klossiella equi; Sarcocystis spp. Typical parasites infecting swine include Eimeria bebliecki, Eimeria scabra, Isospora suis, Giardia spp.; Balantidium coli, Entamoeba histolytica; Toxoplasma gondii and Sarcocystis spp., and Trichinella spiralis. The major parasites of dairy and beef cattle include Eimeria spp., Cryptosporidium sp., Giardia sp., Toxoplasma gondii; Babesia bovis (RBC), Babesia bigemina (RBC), Trypanosoma spp. (plasma), Theileria spp. (RBC); Theileria parva (lymphocytes); Tritrichomonas foetus; and Sarcocystis spp. The major parasites of raptors include Trichomonas gallinae; Coccidia (Eimeria spp.); Plasmodium relictum, Leucocytozoon danilewskyi (owls), Haemoproteus spp., Trypanosoma spp.; Histomonas; Cryptosporidium meleagridis, Cryptosporidium baileyi, Giardia, Eimeria; Toxoplasma. Typical parasites infecting sheep and goats include Eimeria spp., Cryptosporidium sp., Giardia sp.; Toxoplasma gondii; Babesia spp. (RBC), Trypanosoma spp. (plasma), Theileria spp. (RBC); and Sarcocystis spp. Typical parasitic infections in poultry include coccidiosis caused by Eimeria acervulina, E. necatrix, E. tenella, Isospora spp. and Eimeria truncata; histomoniasis, caused by Histomonas meleagridis and Histomonas gallinarum; trichomoniasis caused by Trichomonas gallinae; and hexamitiasis caused by Hexamita meleagridis. Poultry can also be infected Emeria maxima, Emeria meleagridis, Eimeria adenoeides, Eimeria meleagrimitis, Cryptosporidium, Eimeria brunetti, Emeria adenoeides, Leucocytozoon spp., Plasmodium spp., Hemoproteus meleagridis, Toxoplasma gondii and Sarcocystis.

Parasitic infections also pose serious problems in laboratory research settings involving animal colonies. Some examples of laboratory animals intended to be treated, or in which parasite infection is sought to be prevented, by the methods of the invention include mice, rats, rabbits, guinea pigs, nonhuman primates, as well as the aforementioned swine and sheep.

Typical parasites in mice include Leishmania spp., Plasmodium berghei, Plasmodium yoelii, Giardia muris, Hexamita muris; Toxoplasma gondii, Trypanosoma duttoni (plasma); Klossiella muris; Sarcocystis spp. Typical parasites in rats include Giardia muris, Hexamita muris; Toxoplasma gondii; Trypanosoma lewisi (plasma); Trichinella spiralis; Sarcocystis spp. Typical parasites in rabbits include Eimeria sp.; Toxoplasma gondii; Nosema cuniculi; Eimeria stiedae, Sarcocystis spp. Typical parasites of the hamster include Trichomonas spp.; Toxoplasma gondii; Trichinella spiralis; Sarcocystis spp. Typical parasites in the guinea pig include Balantidium caviae; Toxoplasma gondii; Klossiella caviae; Sarcocystis spp.

The methods of the invention can also be applied to the treatment and/or prevention of parasitic infection in dogs, cats, birds, fish and ferrets. Typical parasites of birds include Trichomonas gallinae; Eimeria spp., Isospora spp., Giardia; Cryptosporidium; Sarcocystis spp., Toxoplasma gondii, Haemoproteus/Parahaemoproteus, Plasmodium spp., Leucocytozoon/Akiba, Atoxoplasma, Trypanosoma spp. Typical parasites infecting dogs include Trichinella spiralis, Isopora spp., Sarcocystis spp., Cryptosporidium spp., Hammondia spp., Giardia duodenalis (canis); Balantidium coli, Entamoeba histolytica; Hepatozoon canis; Toxoplasma gondii, Trypanosoma cruzi; Babesia canis, Leishmania amastigotes, Neospora caninum. Typical parasites infecting feline species include Isospora spp., Toxoplasma gondii, Sarcocystis spp., Hammondia hammondi, Besnoitia spp., Giardia spp.; Entamoeba histolytica; Hepatozoon canis, Cytauxzoon sp., Cytauxzoon sp., Cytauxzoon sp. (red cells, RE cells). Typical parasites infecting fish include Hexamita spp., Eimeria spp.; Cryptobia spp., Nosema spp., Myxosoma spp., Chilodonella spp., Trichodina spp.; Plistophora spp., Myxosoma Henneguya; Costia spp., Ichthyophithirius spp., and Oodinium spp.

Typical parasites of wild mammals include Giardia spp. (carnivores, herbivores), Isospora spp. (carnivores), Eimeria spp. (carnivores, herbivores); Theileria spp. (herbivores), Babesia spp. (carnivores, herbivores), Trypanosoma spp. (carnivores, herbivores); Schistosoma spp. (herbivores); Fasciola hepatica (herbivores), Fascioloides magna (herbivores), Fasciola gigantica (herbivores), Trichinella spiralis (carnivores, herbivores). Parasitic infections in zoos can also pose serious problems. Typical parasites of the bovidae family (blesbok, antelope, banteng, eland, gaur, impala, klipspringer, kudu, gazelle) include Eimeria spp. Typical parasites in the pinnipedae family (seal, sea lion) include Eimeria phocae. Typical parasites in the camelidae family (camels, llamas) include Eimeria spp. Typical parasites of the giraffidae family (giraffes) include Eimeria spp. Typical parasites in the elephantidae family (African and Asian) include Fasciola spp. Typical parasites of lower primates (chimpanzees, orangutans, apes, baboons, macaques, monkeys) include Giardia sp.; Balantidium coli, Entamoeba histolytica, Sarcocystis spp., Toxoplasma gondii; Plasmodim spp. (RBC), Babesia spp. (RBC), Trypanosoma spp. (plasma), Leishmania spp. (macrophages).

In some cases it is desirable to administer an antigen with the oil-in-water and nucleic acid composition and in other cases no antigen is delivered. The antigen, if used, is preferably a microbial antigen. Microbial antigens include, but are not limited to, cells, cell extracts, proteins, polypeptides, peptides, polysaccharides, polysaccharide conjugates, peptide and non-peptide mimics of polysaccharides and other molecules, small molecules, lipids, glycolipids, and carbohydrates that occur naturally in an infectious agent. In some embodiments, the antigens may also be non-naturally occurring agents that comprise a region of a naturally occurring antigen or that mimic a naturally occurring antigen. Many microbial antigens, however, are protein or polypeptide in nature, as proteins and polypeptides are generally more antigenic than carbohydrates or fats.

Methods for administering an antigen to a subject are well-known in the art, and include intramuscular, intravenous, oral, transdermal, mucosal, intranasal, intratracheal, or subcutaneous administration delivery. In preferred embodiments of the invention, however, the antigen is delivered by the same route as the oil-in-water and immunostimulatory nucleic acid combination (i.e., it is delivered to an external surface such as the skin or mucosa, and preferably the external mucosa).

In some preferred embodiments, the antigen is not conjugated to the immunostimulatory nucleic acid.

The term “substantially purified” as used herein refers to a molecular species that is substantially free of other proteins, lipids, carbohydrates or other materials with which it is naturally associated. One skilled in the art can purify polypeptides, e.g. antigens, using standard techniques for protein purification. The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the polypeptide can also be determined by amino-terminal amino acid sequence analysis.

The microbial antigen, if administered and if it is a polypeptide, may be in the form of a polypeptide when administered to the subject or it may be encoded by a nucleic acid vector. If the nucleic acid vector is administered to the subject the protein is expressed in vivo. Minor modifications of the primary amino acid sequences of polypeptide microbial antigens may also result in a polypeptide which has substantially equivalent antigenic activity, as compared to the unmodified counterpart polypeptide. Such modifications may be deliberate, as by site-directed mutagenesis, or may be spontaneous. Thus, nucleic acids having such modifications are also encompassed. When an antigen that is encoded by a nucleic acid vector is administered, the immunostimulatory nucleic acid is not the same plasmid or expression vector containing the antigen. In some important embodiments, the antigen is not provided to the subject in the form of a nucleic acid vector. Accordingly, as used herein, such an antigen is referred to as a non-nucleic acid antigen. This latter category of antigens can be peptide or non-peptide in nature but is not a nucleic acid that encodes an antigen.

The nucleic acid encoding the antigen is operatively linked to a gene expression sequence that directs the expression of the protein within a eukaryotic cell. The “gene expression sequence” is any regulatory nucleotide sequence, such as a promoter sequence or promoter-enhancer combination, which facilitates the efficient transcription and translation of the protein to which it is operatively linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPTR), adenosine deaminase, pyruvate kinase, b-actin promoter and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

The emulsion/nucleic acid composition is also useful for treating and preventing cancer when administered topically. Present cancer treatments are too often ineffective as well as being associated with a high degree of patient morbidity, most probably due to a lack of toxic specificity for tumor cells. The compositions of the invention provide a more effective treatment of cancer by promoting an enhanced immune response. The immune response may be antigen specific or an innate immune response (non-antigen specific). In some instances, the emulsion/nucleic acid composition is synergistic, resulting in greater than additive effects than would otherwise be expected using the agents separately, or using the nucleic acids in other formulations.

Thus, in one aspect, the invention provides a method for treating or preventing cancer which involves the administration of some forms of immunostimulatory nucleic acid together with an oil-in-water emulsion in an effective amount to prevent or treat the cancer to a subject having cancer or a subject at risk of developing cancer, particularly when administered topically.

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

The term “tumor” is generally used to mean a solid mass cancer. The method of the invention can be used to treat cancers such as but not limited to sarcoma, carcinoma, fibroma, leukemia, lymphoma, melanoma, myeloma, neuroblastoma, rhabdomyosarcoma, retinoblastoma, and glioma as well as each of the other tumors described herein. Particular examples of cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas. In preferred embodiments, the cancer is one that can be treated by topical delivery of a therapeutic agent or one that exists, even if only partially, at a topical surface. The topical surface can include the skin, the scalp, the eyes, the oral cavity, the nasal cavity, the vagina, the rectum and the like. Accordingly, the cancers to be prevented or treated include oral cancer, larynx cancer, esophageal cancer, cervical cancer, ovarian cancer, rectal cancer, skin cancer such as basal cell carcinoma or melanoma, and the like. In important embodiments, the cancer is a basal cell carcinoma or a melanoma or a cervical cancer.

A cancer cell is a cell that divides and reproduces abnormally due to a loss of normal growth control. Cancer cells almost always arise from at least one genetic mutation. In some instances, it is possible to distinguish cancer cells from their normal counterparts based on profiles of expressed genes and proteins, as well as to the level of their expression. Genes commonly affected in cancer cells include oncogenes, such as ras, neu/HER2/erbB, myb, myc and abl, as well as tumor suppressor genes such as p53, Rb, DCC, PET and WT. Cancer-related mutations in some of these genes leads to a decrease in their expression or a complete deletion. In others, mutations cause an increase in expression or the expression of an activated variant of the normal counterpart. Genetic mutations in cancer cells can be targets of therapeutic formulations in some instances. For example, some medicaments target proteins which are thought to be necessary for cancer cell survival and division, such as cell cycle proteins (e.g., cyclin dependent kinases), telomerase and telomerase associated proteins, and tumor suppressor proteins, many of which are upregulated, or unregulated, in cancer cells.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

The methods and compositions provided herein can be used to prevent and treat cancer in human and non-human subjects. Cancer is one of the leading causes of death in companion animals (i.e., cats and dogs). Cancer usually strikes older animals which, in the case of house pets, have become integrated into the family. Forty-five % of dogs older than 10 years of age, are likely to succumb to the disease. The most common treatment options include surgery, chemotherapy and radiation therapy. Others treatment modalities which have been used with some success are laser therapy, cryotherapy, hyperthermia and immunotherapy. The choice of treatment depends on type of cancer and degree of dissemination. Unless the malignant growth is confined to a discrete area in the body, it is difficult to remove only malignant tissue without also affecting normal cells.

Malignant disorders commonly diagnosed in dogs and cats include but are not limited to lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma. Other neoplasias in dogs include genital squamous cell carcinoma, transmissable veneral tumor, testicular tumor, seminoma, Sertoli cell tumor, hemangiopericytoma, histiocytoma, chloroma (granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma. Additional malignancies diagnosed in cats include follicular lymphoma, intestinal lymphosarcoma, fibrosarcoma and pulmonary squamous cell carcinoma. The ferret, an ever-more popular house pet, is known to develop insulinoma, lymphoma, sarcoma, neuroma, pancreatic islet cell tumor, gastric MALT lymphoma and gastric adenocarcinoma.

Neoplasias affecting agricultural livestock include leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lumphadenitis (CLA): chronic, infectious, contagious disease of sheep and goats caused by the bacterium Corynebacterium pseudotuberculosis, and contagious lung tumor of sheep caused by jaagsiekte.

In one aspect, a method for treating cancer is provided which involves administering the compositions of the invention to a subject having cancer. A “subject having cancer” is a subject that has been diagnosed with a cancer. In some embodiments, the subject has a cancer type characterized by a solid mass cancer (i.e., a tumor). The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue.

In the case of external surface cancers (i.e., those that involve external surfaces such as the skin and mucosa), such tumor masses most probably are visually apparent and may not be diagnosed through palpitation methods. Molecular and phenotypic analysis of cancer cells within a tissue will usually confirm if the cancer is endogenous to the tissue or if the lesion is due to metastasis from another site.

With respect to the prophylactic treatment methods, the invention is aimed at administering the compositions of the invention to a subject at risk of developing cancer. A subject at risk of developing a cancer is one who has a high probability of developing cancer. These subjects include, for instance, subjects having a genetic abnormality, the presence of which has been demonstrated to have a correlative relation to a higher likelihood of developing a cancer. Subjects exposed to cancer causing agents such as tobacco, asbestos, or other chemical toxins are also subjects at risk of developing cancers used herein. When a subject at risk of developing a cancer is administered an emulsion/nucleic acid formulation topically, the subject will be able to mount a continuous immune response against the cancer. An antigen may also be used to provoke a cancer specific immune response. If a tumor begins to form in the subject, the subject will develop a specific immune response against one or more of the cancer antigens. This aspect of the invention is particularly advantageous when the antigen to which the subject will be exposed is known. For instance, subjects employed in certain trades which are exposed to cancer-causing agents on an ongoing basis would be ideal subjects for treatment according to the invention, particularly because cancer-causing agents usually preferentially target a specific organ or tissue. For example, many air borne, or inhaled, carcinogens such as tobacco smoke and asbestos have been associated with lung cancer. The methods in which a subject is passively exposed to an carcinogen can be particularly dependent on timing of the administration of the immunostimulatory nucleic acid and the therapeutic formulation, preferably in the form of a cancer vaccine (e.g., a cancer antigen). For instance, in a subject at risk of developing a cancer, the subject may be administered the immunostimulatory nucleic acid and the cancer vaccine containing a cancer antigen on a regular basis when that risk is greatest, i.e., after exposure to a cancer causing agent.

As used herein, “treating cancer” includes preventing the development of a cancer, reducing the symptoms of cancer, and/or inhibiting the growth of an established cancer.

The emulsion/nucleic acid formulation may also be administered in combination with a cancer medicament. As used herein, a “cancer medicament” refers to a agent which is administered to a subject for the purpose of treating a cancer. In other aspects, the cancer medicament is administered to a subject at risk of developing a cancer for the purpose of reducing the risk of developing the cancer. Cancer medicaments embrace such categories as chemotherapeutic agents, immunotherapeutic agents, cancer vaccines, hormone therapy, and biological response modifiers. Cancer medicaments also include agents which are administered to a subject in order to reduce the symptoms of a cancer, rather than to reduce the tumor or cancer burden (i.e., the number of cancer or tumor cells) in a subject. One example of this latter type of cancer medicament is a blood transfusion which is administered to a subject having cancer in order to maintain red blood cell and/or platelet levels within a normal range. As an example, in the absence of such transfusion, cancer patients with below normal levels of platelets are at risk of uncontrolled bleeding.

As used herein, a cancer antigen is broadly defined as an antigen expressed by a cancer cell. Preferably, the antigen is expressed at the cell surface of the cancer cell. Even more preferably, the antigen is one which is not expressed by normal cells, or at least not expressed to the same level as in cancer cells. For example, some cancer antigens are normally silent (i.e., not expressed) in normal cells, some are expressed only at certain stages of differentiation and others are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. The differential expression of cancer antigens in normal and cancer cells can be exploited in order to target cancer cells. As used herein, the terms “cancer antigen” and “tumor antigen” are used interchangeably.

The invention also embraces the prevention or treatment of conditions that are not cancers or infectious diseases. These additional conditions include allergic and non-allergic conditions. These conditions include contact dermatitis, eczema, latex dermatitis, anaphylaxis, allergic rhinitis (hayfever), allergic asthma, atopic dermatitis, psoriasis, allergic contact dermatitis and many types of autoimmune disease.

In other aspects of the invention, the emulsion/nucleic acid formulation allows for the administration of lower doses of antigen than could ordinarily be administered to produce an effective antigen specific immune response. Thus, the immunostimulatory nucleic acids allow for the administration of lower, sub-therapeutic doses of the antigen, but with higher efficacy than would otherwise be achieved using such low doses. As one example, by administering an immunostimulatory nucleic acid with a dose of antigen that if otherwise used in combination with a conventional adjuvant such as alum would be ineffective, it is possible to achieve an effective immune response against the antigen even though one of skill in the art would not have expected that dose of antigen to provide a therapeutic benefit (i.e., a sub-therapeutic dose).

An “immunostimulatory nucleic acid” as used herein is any nucleic acid containing an immunostimulatory motif or backbone that induces an immune response. The immune response may be characterized as, but is not limited to, a Th1-type immune response or a Th2-type immune response. Such immune responses are defined by cytokine and antibody production profiles which are elicited by the activated immune cells.

Helper CD4⁺, and in some instances also CD8⁺, T cells are characterized as Th1 and Th2 cells in both murine and human systems, depending on their cytokine production profiles (Romagnani, 1991, Immunol Today 12: 256-257, Mosmann, 1989, Annu Rev Immunol, 7: 145-173). Th1 cells produce interleukin 2 (IL-2), IL-12, tumor necrosis factor (TNFα) and interferon gamma (IFN-γ) and they are responsible primarily for cell-mediated immunity such as delayed type hypersensitivity. The cytokines that are induced by administration of immunostimulatory nucleic acids are predominantly of the Th1 class. The types of antibodies associated with a Th1 response are generally more protective because they have high neutralization and opsonization capabilities. Th2 cells produce IL-4, IL-5, IL-6, IL-9, IL-10 and IL-13 and are primarily involved in providing optimal help for humoral immune responses such as IgE and IgG4 antibody isotype switching (Mosmann, 1989, Annu Rev Immunol, 7: 145-173). Th2 responses involve predominantly antibodies that have less protective effects against infection.

The terms “nucleic acid” and “oligonucleotide” are used interchangeably to mean multiple nucleotides (i.e., molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. Nucleic acids include vectors, e.g., plasmids, as well as oligonucleotides. However, as used herein, the efficacy of the immunostimulatory nucleic acid devices from its ability to directly activate certain immune cells without expression from the nucleic acid. Thus, even if the nucleic acid encodes a peptide or protein, its therapeutic or prophlyactic immunostimulatory activity is independent of the encoded peptide or protein and will occur even if there is no expression from the nucleic acid. Nucleic acid molecules can be obtained from existing nucleic acid sources (e.g., genomic or cDNA, referred to as isolated nucleic acids), but are preferably synthetic (e.g. produced by oligonucleotide synthesis).

Immunostimulatory nucleic acids may possess immunostimulatory motifs such as CpG motifs, and poly-G motifs. In some embodiments of the invention, any nucleic acid, regardless of whether it possesses an identifiable motif, can be used in the combination therapy to elicit an immune response. Immunostimulatory backbones include, but are not limited to, phosphate modified backbones, such as phosphorothioate backbones. Immunostimulatory nucleic acids have been described extensively in the prior art and a brief summary of these nucleic acids is presented below.

In some embodiments, a CpG immunostimulatory nucleic acid is used in the methods of the invention. A CpG immunostimulatory nucleic acid is a nucleic acid that contains at least one CG dinucleotide, the C residue of which is unmethylated.

A nucleic acid containing at least one unmethylated CpG dinucleotide is a nucleic acid molecule which contains an unmethylated cytosine in a cytosine-guanine dinucleotide sequence (i.e. “CpG DNA” or DNA containing a 5′ cytosine followed by 3′ guanosine and linked by a phosphate bond) and activates the immune system.

The entire immunostimulatory nucleic acid can be unmethylated or portions may be unmethylated but at least the C of the 5′ CG 3′ must be unmethylated.

In one preferred embodiment the invention provides an immunostimulatory nucleic acid that is a CpG nucleic acid represented by at least the formula:

5′X₁X₂CGX₃X₄3′

wherein X₁, X₂, X₃, and X₄ are nucleotides. In one embodiment X₂ is adenine, guanine, cytosine, or thymine. In another embodiment X₃ is cytosine, guanine, adenine, or thymine. In other embodiments X₂ is adenine, guanine, or thymine and X₃ is cytosine, adenine, or thymine.

In another embodiment the immunostimulatory nucleic acid is an isolated CpG nucleic acid represented by at least the formula:

5′N₁X₁X₂CGX₃X₄N₂3′

wherein X₁, X₂, X₃, and X₄ are nucleotides and N is any nucleotide and N₁ and N₂ are nucleic acid sequences composed of from about 0-25 N's each. In one embodiment X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, ApT, TpG, ApG, CpG, TpC, ApC, CpC, TpA, ApA, and CpA. Preferably X₁X₂ are GpA or GpT and X₃X₄ are TpT. In other embodiments X₁ or X₂ or both are purines and X₃ or X₄ or both are pyrimidines or X₁X₂ are GpA and X₃ or X₄ or both are pyrimidines. In another preferred embodiment X₁X₂ are nucleotides selected from the group consisting of: TpA, ApA, ApC, ApG, and GpG. In yet another embodiment X₃X₄ are nucleotides selected from the group consisting of: TpT, TpA, TpG, ApA, ApG, ApC, and CpA. X₁X₂ in another embodiment are nucleotides selected from the group consisting of: TpT, TpG, ApT, GpC, CpC, CpT, TpC, GpT and CpG.

In another preferred embodiment the immunostimulatory nucleic acid has the sequence 5′TCN₁TX₁X₂CGX₃X₄3′ (SEQ ID NO:157). The immunostimulatory nucleic acids of the invention in some embodiments include X₁X₂ selected from the group consisting of GpT, GpG, GpA and ApA and X₃X₄ is selected from the group consisting of TpT, CpT and TpC.

CpG immunostimulatory nucleic acids are known to stimulate Th1-type immune responses. These CpG sequences, while relatively rare in human DNA are commonly found in the DNA of infectious organisms such as bacteria. The human immune system has apparently evolved to recognize CpG sequences as an early warning sign of infection and to initiate an immediate and powerful immune response against invading pathogens without causing adverse reactions frequently seen with other immune stimulatory agents. Thus CpG immunostimulatory nucleic acids, relying on this innate immune defense mechanism can utilize a unique and natural pathway for immune therapy. The effects of CpG nucleic acids on immune modulation have been described extensively in U.S. Pat. No. 6,194,388, and published patent applications, such as PCT US95/01570, PCT/US97/19791, PCT/US98/03678, PCT/US98/10408, PCT/US98/04703, PCT/US99/07335, and PCT/US99/09863. The entire contents of each of these issued patents and patent applications are hereby incorporated by reference. CpG immunostimulatory nucleic acids are also described in U.S. Patent Application 60/404,820 filed Aug. 19, 2002; Ser. No. 10/161,229 filed Jun. 3, 2002, and Ser. No. 10/224,523 filed Aug. 19, 2002, the entire contents of which are incorporated herein by reference.

In one embodiment, the immunostimulatory nucleic acids are referred to as class A nucleic acids. These are strong inducers of IFN-α and natural killer (NK) cell activation but relatively poor inducers of B-cell and DC activation. Krieg A M et al. (1995) Nature 374:546-9; Ballas Z K et al. (1996) J Immunol 157:1840-5; Yamamoto S et al. (1992) J Immunol 148:4072-6. Examples of class A immunostimulatory nucleic acid include those that contain at least one unmethylated CpG dinucleotide and which are from about 8-80 bases in length. In one embodiment the unmethylated CpG dinucleotide has a formula:

5′N₁X₁CGX₂N₂3′

wherein at least one nucleotide separates consecutive CpGs; X₁ is adenine, guanine, or thymine; X₂ is cytosine, adenine, or thymine; N is any nucleotide and N₁+N₂ is from about 0-25 nucleotides. In another embodiment the unmethylated CpG dinucleotide has a formula:

5′NX₁X₂CGX₃X₄N3′

wherein at least one nucleotide separates consecutive CpGs; X₁X₂ is selected from the group consisting of TpT, CpT, TpC, and ApT; X₃X₄ is selected from the group consisting of GpT, GpA, ApA and ApT; N is any nucleotide and N₁+N₂ is from about 0-25 nucleotides. In a preferred embodiment N₁ and N₂ of the nucleic acid do not contain a CCGG quadmer or more than one CCG or CGG trimer.

In yet another embodiment the nucleotide of the isolated nucleic acid has a phosphate backbone modification, such as, for example, a phosphorothioate or phosphorodithioate modification. In one embodiment the phosphate backbone modification occurs at the 5′ end of the nucleic acid. Preferably the phosphate backbone modification occurs at the first two internucleotide linkages of the 5′ end of the nucleic acid. According to another embodiment the phosphate backbone modification occurs at the 3′ end of the nucleic acid. Preferably, the phosphate backbone modification occurs at the last five internucleotide linkages of the 3′ end of the nucleic acid.

In one embodiment, the immunostimulatory nucleic acids are referred to as class C nucleic acids. While preferred class A CpG ODN have mixed or chimeric backbones, the class C of combination motif immune stimulatory nucleic acids may have either stabilized, e.g., phosphorothioate, chimeric, or phosphodiester backbones.

In one aspect the invention provides immune stimulatory nucleic acids belonging to the class C of combination motif immune-stimulatory nucleic acids. The B cell stimulatory domain is defined by a formula: 5′ X₁DCGHX₂ 3′. D is a nucleotide other than C. C is cytosine. G is guanine. H is a nucleotide other than G.

X₁ and X₂ are any nucleic acid sequence 0 to 10 nucleotides long. X₁ may include a CG, in which case there is preferably a T immediately preceding this CG. In some embodiments DCG is TCG. X₁ is preferably from 0 to 6 nucleotides in length. In some embodiments X₂ does not contain any poly G or poly A motifs. In other embodiments the immunostimulatory nucleic acid has a poly-T sequence at the 5′ end or at the 3′ end. As used herein, “poly-A” or “poly-T” shall refer to a stretch of four or more consecutive A's or T's respectively, e.g., 5′ AAAA 3′ or 5′ TTTT 3′.

As used herein, “poly-G end” shall refer to a stretch of four or more consecutive G's, e.g., 5′ GGGG 3′, occurring at the 5′ end or the 3′ end of a nucleic acid. As used herein, “poly-G nucleic acid” shall refer to a nucleic acid having the formula 5′ X₁X₂GGGX₃X₄ 3′ wherein X₁, X₂, X₃, and X₄ are nucleotides and preferably at least one of X₃ and X₄ is a G.

Some preferred designs for the B cell stimulatory domain under this formula comprise TTTTTCG, TCG, TTCG, TTTCG, TTTTCG, TCGT, TTCGT, TTTCGT, TCGTCGT.

The second motif of the nucleic acid is referred to as either P or N and is positioned immediately 5′ to X₁ or immediately 3′ to X₂.

N is a B-cell neutralizing sequence that begins with a CGG trinucleotide and is at least 10 nucleotides long. A B-cell neutralizing motif includes at least one CpG sequence in which the CG is preceded by a C or followed by a G (Krieg A M et al. (1998) Proc Natl Acad Sci USA 95:12631-12636) or is a CG containing DNA sequence in which the C of the CG is methylated. As used herein, “CpG” shall refer to a 5′ cytosine (C) followed by a 3′ guanine (G) and linked by a phosphate bond. At least the C of the 5′ CG 3′ must be unmethylated. Neutralizing motifs are motifs which has some degree of immunostimulatory capability when present in an otherwise non-stimulatory motif, but, which when present in the context of other immunostimulatory motifs serve to reduce the immunostimulatory potential of the other motifs.

P is a GC-rich palindrome containing sequence at least 10 nucleotides long. As used herein, “palindrome” and, equivalently, “palindromic sequence” shall refer to an inverted repeat, i.e., a sequence such as ABCDEE′D′C′B′A′ in which A and A′, B and B′, etc., are bases capable of forming the usual Watson-Crick base pairs.

As used herein, “GC-rich palindrome” shall refer to a palindrome having a base composition of at least two-thirds G's and C's. In some embodiments the GC-rich domain is preferably 3′ to the “B cell stimulatory domain”. In the case of a 10-base long GC-rich palindrome, the palindrome thus contains at least 8 G's and C's. In the case of a 12-base long GC-rich palindrome, the palindrome also contains at least 8 G's and C's. In the case of a 14-mer GC-rich palindrome, at least ten bases of the palindrome are G's and C's. In some embodiments the GC-rich palindrome is made up exclusively of G's and C's.

In some embodiments the GC-rich palindrome has a base composition of at least 81 percent G's and C's. In the case of such a 10-base long GC-rich palindrome, the palindrome thus is made exclusively of G's and C's. In the case of such a 12-base long GC-rich palindrome, it is preferred that at least ten bases (83 percent) of the palindrome are G's and C's. In some preferred embodiments, a 12-base long GC-rich palindrome is made exclusively of G's and C's. In the case of a 14-mer GC-rich palindrome, at least twelve bases (86 percent) of the palindrome are G's and C's. In some preferred embodiments, a 14-base long GC-rich palindrome is made exclusively of G's and C's. The C's of a GC-rich palindrome can be unmethylated or they can be methylated.

In general this domain has at least 3 Cs and Gs, more preferably 4 of each, and most preferably 5 or more of each. The number of Cs and Gs in this domain need not be identical. It is preferred that the Cs and Gs are arranged so that they are able to form a self-complementary duplex, or palindrome, such as CCGCGCGG. This may be interrupted by As or Ts, but it is preferred that the self-complementarity is at least partially preserved as for example in the motifs CGACGTTCGTCG (SEQ ID NO:158) or CGGCGCCGTGCCG (SEQ ID NO:159). When complementarity is not preserved, it is preferred that the non-complementary base pairs be TG. In a preferred embodiment there are no more than 3 consecutive bases that are not part of the palindrome, preferably no more than 2, and most preferably only 1. In some embodiments the GC-rich palindrome includes at least one CGG trimer, at least one CCG trimer, or at least one CGCG tetramer. In other embodiments the GC-rich palindrome is not CCCCCCGGGGGG (SEQ ID NO:160) or GGGGGGCCCCCC (SEQ ID NO:161), CCCCCGGGGG (SEQ ID NO:162) or GGGGGCCCCC (SEQ ID NO:163).

At least one of the G's of the GC rich region may be substituted with an inosine (I). In some embodiments P includes more than one I.

In certain embodiments the immunostimulatory nucleic acid has one of the following formulas 5′ NX₁DCGHX₂ 3′, 5′ X₁DCGHX₂N 3′, 5′ PX₁DCGHX₂ 3′, 5′ X₁DCGHX₂P 3′, 5′ X₁DCGHX₂PX₃ 3′, 5′ X₁DCGHPX₃ 3′, 5′ DCGHX₂PX₃ 3′, 5′ TCGHX₂PX₃ 3′, 5′ DCGHPX₃ 3′, or 5′ DCGHP 3′.

In other aspects the invention provides immune stimulatory nucleic acids which are defined by a formula: 5′ N₁PyGN₂P 3′. N₁ is any sequence 1 to 6 nucleotides long. Py is a pyrimidine. G is guanine. N₂ is any sequence 0 to 30 nucleotides long. P is a GC-rich palindrome containing sequence at least 10 nucleotides long.

N₁ and N₂ may contain more than 50% pyrimidines, and more preferably more than 50% T. N₁ may include a CG, in which case there is preferably a T immediately preceding this CG. In some embodiments N₁PyG is TCG (such as ODN 5376, which has a 5′ TCGG), and most preferably a TCGN₂, where N₂ is not G.

N₁PyGN₂P may include one or more inosine (I) nucleotides. Either the C or the G in N1 may be replaced by inosine, but the CpI is preferred to the IpG. For inosine substitutions such as IpG, the optimal activity may be achieved with the use of a “semi-soft” or chimeric backbone, where the linkage between the IG or the CI is phosphodiester. N₁ may include at least one CI, TCI, IG or TIG motif.

In certain embodiments N₁PyGN₂ is a sequence selected from the group consisting of TTTTTCG, TCG, TTCG, TTTCG, TTTTCG, TCGT, TTCGT, TTTCGT, and TCGTCGT.

In other aspects the invention provides immune stimulatory nucleic acids which are defined by a formula: 5′ N₁PyG/IN₂P 3′. N₁ is any sequence 1 to 6 nucleotides long. Py is a pyrimidine, G/I refers to single nucleotide which is either a G or an I. G is guanine and I is inosine. N₂ is any sequence 0 to 30 nucleotides long. P is a GC or IC rich palindrome containing sequence at least 10 nucleotides long. In some embodiments N₁PyIN₂ is TCITCITTTT.

Some non-limiting examples of combination motif immune stimulatory nucleic acids, which are described by the formulas above, include the following:

(SEQ ID NO:164)

TCGTCGTTTTCGGCGCGCGCCG,

(SEQ ID NO:165)

TCGTCGTTTTCGGCGGCCGCCG,

(SEQ ID NO:166)

TCGTCGTTTTCGGCGCGCCGCG,

(SEQ ID NO:167)

TCG TCG TTT TCG GCG CCG GCC G,

(SEQ ID NO:168)

TCGTCGTTTTCGGCCCGCGCGG,

(SEQ ID NO:169)

TCG TCG TTT TCG GCG CGC GCC GTT TTT,

(SEQ ID NO:170)

TCC TGA CGT TCG GCG CGC GCC G,

(wherein Z is 5-methylcytosine; SEQ ID NO:171)

TZGTZGTTTTZGGZGZGZGZZG,

(SEQ ID NO:172)

TCCTGACGTTCGGCGCGCGCCC,

(SEQ ID NO:173)

TCG TCG TTT TCG GCG GCC GAC G,

(SEQ ID NO:174)

TCGTCGTTTTCGTCGGCCGCCG,

(SEQ ID NO:175)

TCGTCGTTTTCGACGGCCGCCG,

(SEQ ID NO:176)

TCG TCG TTT TCG GCG GCC GTC G,

(SEQ ID NO:177)

TCGGCGCGCGCCGTCGTCGTTT,

(SEQ ID NO:178)

TCG TCG TTT CGA CGG CCG TCG,

(SEQ ID NO:179)

TCGTCGTTTCGACGATCGTCG,

(SEQ ID NO:180)

TCGTCGTTTCGACGTACGTCG,

(SEQ ID NO:181)

TCGTCGCGACGGCCGTCG,

(SEQ ID NO:182)

TCGTCGCGACGATCGTCG,

(SEQ ID NO:183)

TCGTCGCGACGTACGTCG,

(SEQ ID NO:184)

TCG TTT TTT TCG ACG GCC GTC G,

(SEQ ID NO:185)

TCG TTT TTT TCG ACG ATC GTC G,

(SEQ ID NO:186)

TCG TTT TTT TCG ACG TAC GTC G,

(SEQ ID NO:187)

TIGTIGTTTTCGGCGGCCGCCG,
and

(SEQ ID NO:188)

TCI TCI TTT TCG GCG GCC GCC G.

In still other embodiments, the immunostimulatory nucleic acids are referred to as “soft” or “semi-soft” immunostimulatory nucleic acids. These are immunostimulatory nucleic acid molecule having at least one internal pyrimidine nucleoside-guanosine (YG) dinucleotide and a chimeric backbone, wherein the at least one internal YG dinucleotide has a phosphodiester or phosphodiester-like internucleoside linkage, wherein optionally each additional internal YG dinucleotide has a phosphodiester, phosphodiester-like, or stabilized internucleoside linkage, and wherein all other internucleoside linkages are stabilized. In one embodiment the immunostimulatory nucleic acid comprises a plurality of internal YG dinucleotides each having a phosphodiester or phosphodiester-like internucleoside linkage. In one embodiment every internal YG dinucleotide has a phosphodiester or phosphodiester-like internucleoside linkage.

In one embodiment the immunostimulatory nucleic acid molecule is selected from the group consisting of: *A*C_G*T*C_G*T*T*T*T*C_G*T*C_G*T*T, (SEQ ID NO: 189); G*C_G*T*C_G*A*C_G*T*C_G*A*C_G*C, (SEQ ID NO: 190); G*C_G*T*C_G*T*T*T*T*C_G*T*C_G*C, (SEQ ID NO: 191); T*C*C*A*T_G*A*C_G*T*T*C*C*T_G*A*T*G*C, (SEQ ID NO: 192); T*C*G*T*C*G*T*T*T*T*C*G*T*C_G*T*T, (SEQ ID NO: 193); T*C*G*T*C*G*T*T*T*T*C_G*G*C_G*G*C*C_G*C*C*G, (SEQ ID NO: 194); T*C*G*T*C*G*T*T*T*T*C_G*T*C_G*T*T, (SEQ ID NO: 195); T*C*G*T*C_G*T*T*T*C_G*T*C_G*T*T, (SEQ ID NO: 196); T*C*G*T*C_G*T*T*T*T*C*G*T*C*G*T*T, (SEQ ID NO: 197); T*C*G*T*C_G*T*T*T*T*C*G*T*C_G*T*T, (SEQ ID NO: 198); T*C*G*T*C_G*T*T*T*T*C_G*T*C*G*T*T, (SEQ ID NO: 199); T*C_{—}7*T*C_{—}7*T*T*T*T_G*T*C_G*T*T*T*T_G*T*C_G*T*T, (SEQ ID NO: 200); T*C_{—}7*T*C_G*T*T*T*T_G*T*C_G*T*T*T*T_G*T*C_{—}7*T*T, (SEQ ID NO: 201); T*C_G*C*C_G*T*T*T*T*C_G*G*C_G*G*C*C_G*C*C*G, (SEQ ID NO: 202); T*C_G*T*C*G*T*T*T*T*A*C*G*A*C*G*T*C*G*C*G, (SEQ ID NO: 203); T*C_G*T*C*G*T*T*T*T*A*C*G*A*C*G*T*C*G*T*G, (SEQ ID NO: 204); T*C_G*T*C*G*T*T*T*T*A*C*G*G*C*G*C*C*G*C*G*C*C*G, (SEQ ID NO: 205); T*C_G*T*C*G*T*T*T*T*A*C*G*G*C*G*T*C*G*C*G, (SEQ ID NO: 206); T*C_G*T*C*G*T*T*T*T*A*C*G*G*C*G*T*C*G*C*G*C*C*G, (SEQ ID NO: 207); T*C_G*T*C*G*T*T*T*T*A*C*G*G*C*G*T*C*G*T*G*C*C*G, (SEQ ID NO: 208); T*C_G*T*C*G*T*T*T*T*C*G*G*C*G*C*G*C*G*C*C*G, (SEQ ID NO: 209); T*C_G*T*C*G*T*T*T*T*C*G*T*C*G*T*T, (SEQ ID NO: 210); T*C_G*T*C*G*T*T*T*T*C*G*T*C_G*T*T, (SEQ ID NO: 211); T*C_G*T*C*G*T*T*T*T*C_G*T*C*G*T*T, (SEQ ID NO: 212); T*C_G*T*C*G*T*T*T*T*G*C*G*A*C*G*T*C*G*C*G, (SEQ ID NO: 213); T*C_G*T*C*G*T*T*T*T*T*C*G*A*C*G*T*C*G*A*G, (SEQ ID NO: 214); T*C_G*T*C*G*T*T*T*T*T*C*G*A*C*G*T*C*G*C*G, (SEQ ID NO: 215); T*C_G*T*C_{—}7*T*T*T*T_G*T*C_G*T*T*T*T_{—}7*T*C_G*T*T, (SEQ ID NO: 216); T*C_G*T*C_G*T*T*T*C_G*A*C*G*T*T, (SEQ ID NO: 217); T*C_G*T*C_G*T*T*T*C_G*A*C_G*T*T*T*T*G*T*C_G*T*T, (SEQ ID NO: 218); T*C_G*T*C_G*T*T*T*C_G*T*C_G*A*C_G*T*C_G*T*T*T*C_G*T*C*G, (SEQ ID NO: 219); T*C_G*T*C_G*T*T*T*C_G*T*C_G*A*T, (SEQ ID NO: 220); T*C_G*T*C_G*T*T*T*C_G*T*C_G*A*T*T, (SEQ ID NO: 221); T*C_G*T*C_G*T*T*T*C_G*T*C_G*T, (SEQ ID NO: 222); T*C_G*T*C_G*T*T*T*C_G*T*C_G*T*T, (SEQ ID NO: 223); T*C_G*T*C_G*T*T*T*C_G*T*C_G*T*T*T*C_G*T*C_G*T*T,(SEQ ID NO:224); T*C_G*T*C_G*T*T*T*C_G*T*C_G*T*T*T*T*G*T*C_G*T*T, (SEQ ID NO: 225); T*C_G*T*C_G*T*T*T*G*T*C*G*T*C*G*G*C*G*G*C*C*G*C*C*G, (SEQ ID NO: 226); T*C_G*T*C_G*T*T*T*T*C*G*G*C*G*C*G*C*G*C*C*G, (SEQ ID NO: 227); T*C_G*T*C_G*T*T*T*T*C*G*G*C*G*G*C*C*G*C*C*G, (SEQ ID NO: 228); T*C_G*T*C_G*T*T*T*T*C*G*T*C*G*T*T, (SEQ ID NO: 229); T*C_G*T*C_G*T*T*T*T*C_G*G*C_G*C_G*C_G*C*C*G, (SEQ ID NO: 230); T*C_G*T*C_G*T*T*T*T*C_G*G*C_G*G*C*C_G*C*C*G, (SEQ ID NO: 231); T*C_G*T*C_G*T*T*T*T*C_G*T*C_G*T, (SEQ ID NO: 232); T*C_G*T*C_G*T*T*T*T*C_G*T*C_G*T*T, (SEQ ID NO: 233); T*C_G*T*C_G*T*T*T*T*C_G*T*T_G*T*T, (SEQ ID NO:234); T*C_G*T*C_G*T*T*T*T*G*T*C_G*T*C_G*T*T*T*T, (SEQ ID NO: 235); T*C_G*T*C_G*T*T*T*T*T*T*T*T*C_G*T*C_G*T*T*T*T, (SEQ ID NO: 236); T*C_G*T*C_G*T*T*T*T*T_G*T*C_G*T*T, (SEQ ID NO:237); T*C_G*T*C_G*T*T*T*T*T_G*T*T_G*T*T, (SEQ ID NO: 238); T*C_G*T*C_G*T*T*T*T_{—}7*T*C_{—}7*T*T*T*T_G*T*C_G*T*T, (SEQ ID NO: 239); T*C_G*T*C_G*T*T*T*T_G*A*C_G*T*T, (SEQ ID NO: 240); T*C_G*T*C_G*T*T*T*T_G*A*C_G*T*T*T*T, (SEQ ID NO: 241); T*C_G*T*C_G*T*T*T*T_G*A*C_G*T*T*T*T*G*T*C*G*T*T, (SEQ ID NO: 242); T*C_G*T*C_G*T*T*T*T_G*A*C_G*T*T*T*T*G*T*C_G*T*T, (SEQ ID NO: 243); T*C_G*T*C_G*T*T*T*T_G*T*C_G*T*T, (SEQ ID NO:244); T*C_G*T*C_G*T*T*T*T_G*T*C_G*T*T*T*T*G*T*C_G*T*T, (SEQ ID NO: 245); T*C_G*T*C_G*T*T*T*T_G*T*C_G*T*T*T*T_{—}7*T*C_{—}7*T*T, (SEQ ID NO: 246); T*C_G*T*C_G*T*T*T*T_G*T*C_G*T*T*T*T_G*T*C_G*T*T, (SEQ ID NO: 247); T*C_G*T*C_G*T*T*T*U_G*T*C_G*T*T*T, (SEQ ID NO: 248); T*C_G*T*C_G*T*T*T*U_G*T*C_G*T*T*T*T_G*T*C_G*T*T, (SEQ ID NO: 249); T*C_G*T*C_G*T*T*T_G*C_G*T*C_G*T, (SEQ ID NO:250); T*C_G*T*C_G*T*T*T_G*C_G*T*C_G*T*T, (SEQ ID NO: 251); T*C_G*T*C_G*T*T*T_G*T*C_G*T, (SEQ ID NO: 252); T*C_G*T*C_G*T*T*T_G*T*C_G*T*T, (SEQ ID NO: 253); T*C_G*T*C_G*U*U*U*C_G*T*C_G*U*U*U*U_G*T*C_G*T*T, (SEQ ID NO: 254); T*C_G*T*T*T*T*G*T*C_G*T*T*T*T, (SEQ ID NO: 255); T*C_G*T*T*T*T*G*T*C_G*T*T*T*T*T*T*T*T, (SEQ ID NO: 256); T*C_G*T*T*T*T*T*T*T*T*C_G*T*T*T*T, (SEQ ID NO: 257); T*C_G*T*T_G*T*T*T*T*C_G*T*C_G*T*T, (SEQ ID NO: 258); T*C_G*T*T_G*T*T*T*T*C_G*T*T_G*T*T, (SEQ ID NO: 259); T*C_G*T*T_G*T*T*T*T*T_G*T*C_G*T*T, (SEQ ID NO: 260); T*C_G*T*T_G*T*T*T*T*T_G*T*T_G*T*T, (SEQ ID NO: 261); T*C_G*U*C_G*T*T*T*T_G*T*C_G*T*T*T*U_G*U*C_G*T*T, (SEQ ID NO: 262); T*G*T*C_G*T*T*G*T*C_G*T*T*G*T*C_G*T*T*G*T*C_G*T*T, (SEQ ID NO: 263); T*G*T*C_G*T*T*G*T*C_G*T*T_G*T*C_G*T*T_G*T*C_G*T*T, (SEQ ID NO: 264); T*G*T*C_G*T*T*T*C_G*T*C_G*T*T, (SEQ ID NO: 265); T*G*T*C_G*T*T*T*T*G*T*C_G*T*T, (SEQ ID NO: 266); T*T*A*G*T*T*C_G*T*A*G*T*T*C*T*T*C_G*T*T, (SEQ ID NO: 267); T*T*C_G*T*C_G*T*T*T*C_G*T*C_G*T*T, (SEQ ID NO: 268); T*T*C_G*T*C_G*T*T*T*C_G*T*C_G*T*T*T, (SEQ ID NO: 269); T*T*C_G*T*C_G*T*T*T*T_G*T*C_G*T*T, (SEQ ID NO: 270); T*T*C_G*T*T*C*T*T*A*G*T*T*C_G*T*A*G*T*T, (SEQ ID NO: 271); T*T*T*C_G*A*C_G*T*C_G*T*T*T, (SEQ ID NO: 272); T*T*T*T*C_G*T*C_G*T*T*T*T*G*T*C_G*T*C_G*T, (SEQ ID NO: 273); T*T*T*T*C_G*T*C_G*T*T*T*T*G*T*C_G*T*C_G*T*T*T*T, (SEQ ID NO:274); T*T*T*T*C_G*T*C_G*T*T*T*T*T*T*T*T*C_G*T*C_G*T, (SEQ ID NO: 275); T*T*T*T*C_G*T*C_G*T*T*T*T*T*T*T*T*C_G*T*C_G*T*T*T*T, (SEQ ID NO:276); T*T*T*T*C_G*T*C_G*T*T*T*T_G*T*C_G*T*C_G*T*T*T*T, (SEQ ID NO: 277); T*T*T*T*C_G*T*T*T*T*G*T*C_G*T, (SEQ ID NO: 278); T*T*T*T*C_G*T*T*T*T*G*T*C_G*T*T*T*T, (SEQ ID NO: 279); T*T*T*T*C_G*T*T*T*T*T*T*T*T*C_G*T, (SEQ ID NO: 280); T*T*T*T*C_G*T*T*T*T*T*T*T*T*C_G*T*T*T*T, (SEQ ID NO: 281); T*T*T*T*C_G_T*T*T*T_G*T*C_G*T*T*T*T, (SEQ ID NO: 282); T*T*T*T*T*T*T*T*C_G*T*T*T*T*G*T*C_G*T, (SEQ ID NO: 283); T*T_G*T*C_G*T*T*T*T*C_G*T*C_G*T*T, (SEQ ID NO: 284); T*T_G*T*C_G*T*T*T*T*C_G*T*T_G*T*T, (SEQ ID NO: 285); T*T_G*T*C_G*T*T*T*T*T_G*T*C_G*T*T, (SEQ ID NO: 286); and T*T_G*T*C_G*T*T*T*T*T_G*T*T_G*T*T, (SEQ ID NO: 287); wherein * represents phosphorothioate, _{—} represents phosphodiester, U represents 2′-deoxyuracil, and 7 represents 7-deazaguanine.

In one embodiment the immunostimulatory nucleic acid molecule is selected from the group consisting of: T*C_G*T*C_G*T*T*T*T_G*T*C_G*T*T*T*G*T*C_G*T*T, (SEQ ID NO: 288); T*C_G*T*C_G*T*T*T*T_G*T*C_G*T*T, (SEQ ID NO: 289); T*C_G*T*C_G*T*T*T*C_G*T*C_G*T*T, (SEQ ID NO: 290); T*G*T*C_G*T*T*G*T*C_G*T*T_G*T*C_G*T*T_G*T*C_G*T*T, (SEQ ID NO: 291); and T*C_G*T*C_G*T*T*T*T*C*G*G*C*G*G*C*C*G*C*C*G, (SEQ ID NO: 292); wherein * represents phosphorothioate and _{—} represents phosphodiester.

In another aspect the invention provides an immunostimulatory nucleic acid molecule comprising a chimeric backbone and at least one sequence N₁YGN₂, wherein independently for each sequence N₁YGN₂ YG is an internal pyrimidine nucleoside-guanosine (YG) dinucleotide, N₁ and N₂ are each, independent of the other, any nucleotide, and wherein for the at least one sequence N₁YGN₂ and optionally for each additional sequence N₁YGN₂: the YG dinucleotide has a phosphodiester or phosphodiester-like internucleoside linkage, and N₁ and Y are linked by a phosphodiester or phosphodiester-like internucleoside linkage when N₁ is an internal nucleotide, G and N₂ are linked by a phosphodiester or phosphodiester-like internucleoside linkage when N₂ is an internal nucleotide, or N₁ and Y are linked by a phosphodiester or phosphodiester-like internucleoside linkage when N₁ is an internal nucleotide and G and N₂ are linked by a phosphodiester or phosphodiester-like internucleoside linkage when N₂ is an internal nucleotide, wherein all other internucleoside linkages are stabilized.

In one embodiment the immunostimulatory nucleic acid comprises a plurality of the sequence N₁YGN₂, wherein for each sequence N₁YGN₂: the YG dinucleotide has a phosphodiester or phosphodiester-like internucleoside linkage, and N₁ and Y are linked by a phosphodiester or phosphodiester-like internucleoside linkage when N₁ is an internal nucleotide, G and N₂ are linked by a phosphodiester or phosphodiester-like internucleoside linkage when N₂ is an internal nucleotide, or N₁ and Y are linked by a phosphodiester or phosphodiester-like internucleoside linkage when N₁ is an internal nucleotide and G and N₂ are linked by a phosphodiester or phosphodiester-like internucleoside linkage when N₂ is an internal nucleotide.

In one embodiment the immunostimulatory nucleic acid molecule is selected from the group consisting of:

(SEQ ID NO: 293)

T*C_G*T*C_G*T*T*T*T*G*T*C_G*T*T*T*T*G*T*C_G_T*T,;

(SEQ ID NO: 294)

T*C_G*T*C_G*T*T*T*T*G*T*C_G*T*T*T*T*G*T_C_G*T*T,;

(SEQ ID NO: 295)

T*C_G*T*C_G*T*T*T*T*G*T*C_G*T*T*T*T*G*T_C_G_T*T,;

(SEQ ID NO: 296)

T*C_G*T*C_G*T*T*T*T*G*T*C_G_T*T*T*T*G*T*C_G*T*T,;

(SEQ ID NO: 297)

T*C_G*T*C_G*T*T*T*T*G*T*C_G_T*T*T*T*G*T*C_G_T*T,;

(SEQ ID NO: 298)

T*C_G*T*C_G*T*T*T*T*G*T*C_G_T*T*T*T*G*T_C_G*T*T,;

(SEQ ID NO: 299)

T*C_G*T*C_G*T*T*T*T*G*T*C_G_T*T*T*T*G*T_C_G_T*T,;

(SEQ ID NO: 300)

T*C_G*T*C_G*T*T*T*T*G*T_C_G*T*T*T*T*G*T*C_G*T*T,;

(SEQ ID NO: 301)

T*C_G*T*C_G*T*T*T*T*G*T_C_G*T*T*T*T*G*T*C_G_T*T,;

(SEQ ID NO: 302)

T*C_G*T*C_G*T*T*T*T*G*T_C_G*T*T*T*T*G*T_C_G*T*T,;

(SEQ ID NO: 303)

T*C_G*T*C_G*T*T*T*T*G*T_C_G*T*T*T*T*G*T_C_G_T*T,;

(SEQ ID NO: 304)

T*C_G*T*C_G*T*T*T*T*G*T_C_G_T*T*T*T*G*T*C_G*T*T,;

(SEQ ID NO: 305)

T*C_G*T*C_G*T*T*T*T*G*T_C_G_T*T*T*T*G*T*C_G_T*T,;

(SEQ ID NO: 306)

T*C_G*T*C_G*T*T*T*T*G*T_C_G_T*T*T*T*G*T_C_G*T*T,;

(SEQ ID NO: 307)

T*C_G*T*C_G*T*T*T*T*G*T_C_G_T*T*T*T*G*T_C_G_T*T,;

(SEQ ID NO: 308)

T*C_G*T*C_G_T*T*T*T*G*T*C_G*T*T*T*T*G*T*C_G*T*T,;

(SEQ ID NO: 309);

T*C_G*T*C_G_T*T*T*T*G*T*C_G*T*T*T*T*G*T*C_G_T*T,;

(SEQ ID NO: 310);

T*C_G*T*C_G_T*T*T*T*G*T*C_G*T*T*T*T*G*T_C_G*T*T,;

(SEQ ID NO: 311)

T*C_G*T*C_G_T*T*T*T*G*T*C_G*T*T*T*T*G*T_C_G_T*T,;

(SEQ ID NO: 312)

T*C_G*T*C_G_T*T*T*T*G*T*C_G_T*T*T*T*G*T*C_G*T*T,;

(SEQ ID NO: 313)

T*C_G*T*C_G_T*T*T*T*G*T*C_G_T*T*T*T*G*T*C_G_T*T,;

(SEQ ID NO: 314);

T*C_G*T*C_G_T*T*T*T*G*T*C_G_T*T*T*T*G*T_C_G*T*T,;

(SEQ ID NO: 315);

T*C_G*T*C_G_T*T*T*T*G*T*C_G_T*T*T*T*G*T_C_G_T*T;,

(SEQ ID NO: 316)

T*C_G*T*C_G_T*T*T*T*G*T_C_G*T*T*T*T*G*T*C_G*T*T,;

(SEQ ID NO: 317)

T*C_G*T*C_G_T*T*T*T*G*T_C_G*T*T*T*T*G*T*C_G_T*T,;

(SEQ ID NO: 318)

T*C_G*T*C_G_T*T*T*T*G*T_C_G*T*T*T*T*G*T_C_G*T*T,;

(SEQ ID NO: 319)

T*C_G*T*C_G_T*T*T*T*G*T_C_G*T*T*T*T*G*T_C_G_T*T,;

(SEQ ID NO: 320)

T*C_G*T*C_G_T*T*T*T*G*T_C_G_T*T*T*T*G*T*C_G*T*T,;

(SEQ ID NO: 321)

T*C_G*T*C_G_T*T*T*T*G*T_C_G_T*T*T*T*G*T*C_G_T*T,;

(SEQ ID NO: 322)

T*C_G*T*C_G_T*T*T*T*G*T_C_G_T*T*T*T*G*T_C_G*T*T,;

(SEQ ID NO: 323)

T*C_G*T*C_G_T*T*T*T*G*T_C_G_T*T*T*T*G*T_C_G_T*T,;

(SEQ ID NO: 324)

T*C_G*T_C_G*T*T*T*T*G*T*C_G*T*T*T*T*G*T*C_G*T*T,;

(SEQ ID NO: 325)

T*C_G*T_C_G*T*T*T*T*G*T*C_G*T*T*T*T*G*T*C_G_T*T,;

(SEQ ID NO: 326)

T*C_G*T_C_G*T*T*T*T*G*T*C_G*T*T*T*T*G*T_C_G*T*T,;

(SEQ ID NO: 327)

T*C_G*T_C_G*T*T*T*T*G*T*C_G*T*T*T*T*G*T_C_G_T*T,;

(SEQ ID NO: 328)

T*C_G*T_C_G*T*T*T*T*G*T*C_G_T*T*T*T*G*T*C_G*T*T,;

(SEQ ID NO: 329)

T*C_G*T_C_G*T*T*T*T*G*T*C_G_T*T*T*T*G*T*C_G_T*T,;

(SEQ ID NO: 330)

T*C_G*T_C_G*T*T*T*T*G*T*C_G_T*T*T*T*G*T_C_G*T*T,;

(SEQ ID NO: 331)

T*C_G*T_C_G*T*T*T*T*G*T*C_G_T*T*T*T*G*T_C_G_T*T,;

(SEQ ID NO: 332)

T*C_G*T_C_G*T*T*T*T*G*T_C_G*T*T*T*T*G*T*C_G*T*T,;

(SEQ ID NO: 333)

T*C_G*T_C_G*T*T*T*T*G*T_C_G*T*T*T*T*G*T*C_G_T*T,;

(SEQ ID NO: 334)

T*C_G*T_C_G*T*T*T*T*G*T_C_G*T*T*T*T*G*T_C_G*T*T,;

(SEQ ID NO: 335)

T*C_G*T_C_G*T*T*T*T*G*T_C_G*T*T*T*T*G*T_C_G_T*T,;

(SEQ ID NO: 336)

T*C_G*T_C_G*T*T*T*T*G*T_C_G_T*T*T*T*G*T*C_G*T*T,;

(SEQ ID NO: 337)

T*C_G*T_C_G*T*T*T*T*G*T_C_G_T*T*T*T*G*T*C_G_T*T,;

(SEQ ID NO: 338)

T*C_G*T_C_G*T*T*T*T*G*T_C_G_T*T*T*T*G*T_C_G*T*T,;

(SEQ ID NO: 339)

T*C_G*T_C_G*T*T*T*T*G*T_C_G_T*T*T*T*G*T_C_G_T*T,;

(SEQ ID NO: 340)

T*C_G*T_C_G_T*T*T*T*G*T*C_G*T*T*T*T*G*T*C_G*T*T,;

(SEQ ID NO: 341)

T*C_G*T_C_G_T*T*T*T*G*T*C_G*T*T*T*T*G*T*C_G_T*T,;

(SEQ ID NO: 342)

T*C_G*T_C_G_T*T*T*T*G*T*C_G*T*T*T*T*G*T_C_G*T*T,;

(SEQ ID NO: 343)

T*C_G*T_C_G_T*T*T*T*G*T*C_G*T*T*T*T*G*T_C_G_T*T,;

(SEQ ID NO: 344)

T*C_G*T_C_G_T*T*T*T*G*T*C_G_T*T*T*T*G*T*C_G*T*T,;

(SEQ ID NO: 345)

T*C_G*T_C_G_T*T*T*T*G*T*C_G_T*T*T*T*G*T*C_G_T*T,;

(SEQ ID NO: 346)

T*C_G*T_C_G_T*T*T*T*G*T*C_G_T*T*T*T*G*T_C_G*T*T,;

(SEQ ID NO: 347)

T*C_G*T_C_G_T*T*T*T*G*T*C_G_T*T*T*T*G*T_C_G_T*T,;

(SEQ ID NO: 348)

T*C_G*T_C_G_T*T*T*T*G*T_C_G*T*T*T*T*G*T*C_G*T*T,;

(SEQ ID NO: 349)

T*C_G*T_C_G_T*T*T*T*G*T_C_G*T*T*T*T*G*T*C_G_T*T,;

(SEQ ID NO: 350)

T*C_G*T_C_G_T*T*T*T*G*T_C_G*T*T*T*T*G*T_C_G*T*T,;

(SEQ ID NO: 351)

T*C_G*T_C_G_T*T*T*T*G*T_C_G*T*T*T*T*G*T_C_G_T*T,;

(SEQ ID NO: 352)

T*C_G*T_C_G_T*T*T*T*G*T_C_G_T*T*T*T*G*T*C_G*T*T,;

(SEQ ID NO: 353)

T*C_G*T_C_G_T*T*T*T*G*T_C_G_T*T*T*T*G*T*C_G_T*T,;

(SEQ ID NO: 354)

T*C_G*T_C_G_T*T*T*T*G*T_C_G_T*T*T*T*G*T_C_G*T*T,;

(SEQ ID NO: 355)

T*C_G*T_C_G_T*T*T*T*G*T_C_G_T*T*T*T*G*T_C_G_T*T,;

(SEQ ID NO: 356)

T*C_G_T*C_G*T*T*T*T*G*T*C_G*T*T*T*T*G*T*C_G*T*T,;

(SEQ ID NO: 357)

T*C_G_T*C_G*T*T*T*T*G*T*C_G*T*T*T*T*G*T*C_G_T*T,;

(SEQ ID NO: 358)

T*C_G_T*C_G*T*T*T*T*G*T*C_G*T*T*T*T*G*T_C_G*T*T,;

(SEQ ID NO: 359)

T*C_G_T*C_G*T*T*T*T*G*T*C_G*T*T*T*T*G*T_C_G_T*T,;

(SEQ ID NO: 360)

T*C_G_T*C_G*T*T*T*T*G*T*C_G_T*T*T*T*G*T*C_G*T*T,;

(SEQ ID NO: 361)

T*C_G_T*C_G*T*T*T*T*G*T*C_G_T*T*T*T*G*T*C_G_T*T,;

(SEQ ID NO: 362)

T*C_G_T*C_G*T*T*T*T*G*T*C_G_T*T*T*T*G*T_C_G*T*T,;

(SEQ ID NO: 363)

T*C_G_T*C_G*T*T*T*T*G*T*C_G_T*T*T*T*G*T_C_G_T*T,;

(SEQ ID NO: 364)

T*C_G_T*C_G*T*T*T*T*G*T_C_G*T*T*T*T*G*T*C_G*T*T,;

(SEQ ID NO: 365)

T*C_G_T*C_G*T*T*T*T*G*T_C_G*T*T*T*T*G*T*C_G_T*T,;

(SEQ ID NO: 366)

T*C_G_T*C_G*T*T*T*T*G*T_C_G*T*T*T*T*G*T_C_G*T*T,;

(SEQ ID NO: 367)

T*C_G_T*C_G*T*T*T*T*G*T_C_G*T*T*T*T*G*T_C_G_T*T,;

(SEQ ID NO: 368)

T*C_G_T*C_G*T*T*T*T*G*T_C_G_T*T*T*T*G*T*C_G*T*T,;

(SEQ ID NO: 369)

T*C_G_T*C_G*T*T*T*T*G*T_C_G_T*T*T*T*G*T*C_G_T*T,;

(SEQ ID NO: 370)

T*C_G_T*C_G*T*T*T*T*G*T_C_G_T*T*T*T*G*T_C_G*T*T,;

(SEQ ID NO: 371)

T*C_G_T*C_G*T*T*T*T*G*T_C_G_T*T*T*T*G*T_C_G_T*T,;

(SEQ ID NO: 372)

T*C_G_T*C_G_T*T*T*T*G*T*C_G*T*T*T*T*G*T*C_G*T*T,;

(SEQ ID NO: 373)

T*C_G_T*C_G_T*T*T*T*G*T*C_G*T*T*T*T*G*T*C_G_T*T,;

(SEQ ID NO: 374)

T*C_G_T*C_G_T*T*T*T*G*T*C_G*T*T*T*T*G*T_C_G*T*T,;

(SEQ ID NO: 375)

T*C_G_T*C_G_T*T*T*T*G*T*C_G*T*T*T*T*G*T_C_G_T*T,;

(SEQ ID NO: 376)

T*C_G_T*C_G_T*T*T*T*G*T*C_G_T*T*T*T*G*T*C_G*T*T,;

(SEQ ID NO: 377)

T*C_G_T*C_G_T*T*T*T*G*T*C_G_T*T*T*T*G*T*C_G_T*T,;

(SEQ ID NO: 378)

T*C_G_T*C_G_T*T*T*T*G*T*C_G_T*T*T*T*G*T_C_G*T*T,;

(SEQ ID NO: 379)

T*C_G_T*C_G_T*T*T*T*G*T*C_G_T*T*T*T*G*T_C_G_T*T,;

(SEQ ID NO: 380)

T*C_G_T*C_G_T*T*T*T*G*T_C_G*T*T*T*T*G*T*C_G*T*T,;

(SEQ ID NO: 381)

T*C_G_T*C_G_T*T*T*T*G*T_C_G*T*T*T*T*G*T*C_G_T*T,;

(SEQ ID NO: 382)

T*C_G_T*C_G_T*T*T*T*G*T_C_G*T*T*T*T*G*T_C_G*T*T,;

(SEQ ID NO: 383)

T*C_G_T*C_G_T*T*T*T*G*T_C_G*T*T*T*T*G*T_C_G_T*T,;

(SEQ ID NO: 384)

T*C_G_T*C_G_T*T*T*T*G*T_C_G_T*T*T*T*G*T*C_G*T*T,;

(SEQ ID NO: 385)

T*C_G_T*C_G_T*T*T*T*G*T_C_G_T*T*T*T*G*T*C_G_T*T,;

(SEQ ID NO: 386)

T*C_G_T*C_G_T*T*T*T*G*T_C_G_T*T*T*T*G*T_C_G*T*T,;

(SEQ ID NO: 387)

T*C_G_T*C_G_T*T*T*T*G*T_C_G_T*T*T*T*G*T_C_G_T*T,;

(SEQ ID NO: 388)

T*C_G_T_C_G*T*T*T*T*G*T*C_G*T*T*T*T*G*T*C_G*T*T,;

(SEQ ID NO: 389)

T*C_G_T_C_G*T*T*T*T*G*T*C_G*T*T*T*T*G*T*C_G_T*T,;

(SEQ ID NO: 390)

T*C_G_T_C_G*T*T*T*T*G*T*C_G*T*T*T*T*G*T_C_G*T*T,;

(SEQ ID NO: 391)

T*C_G_T_C_G*T*T*T*T*G*T*C_G*T*T*T*T*G*T_C_G_T*T,;

(SEQ ID NO: 392)

T*C_G_T_C_G*T*T*T*T*G*T*C_G_T*T*T*T*G*T*C_G*T*T,;

(SEQ ID NO: 393)

T*C_G_T_C_G*T*T*T*T*G*T*C_G_T*T*T*T*G*T*C_G_T*T,;

(SEQ ID NO: 394)

T*C_G_T_C_G*T*T*T*T*G*T*C_G_T*T*T*T*G*T_C_G*T*T,;

(SEQ ID NO: 395)

T*C_G_T_C_G*T*T*T*T*G*T*C_G_T*T*T*T*G*T_C_G_T*T,;

(SEQ ID NO: 396)

T*C_G_T_C_G*T*T*T*T*G*T_C_G*T*T*T*T*G*T*C_G*T*T,;

(SEQ ID NO: 397)

T*C_G_T_C_G*T*T*T*T*G*T_C_G*T*T*T*T*G*T*C_G_T*T,;

(SEQ ID NO: 398)

T*C_G_T_C_G*T*T*T*T*G*T_C_G*T*T*T*T*G*T_C_G*T*T,;

(SEQ ID NO: 399)

T*C_G_T_C_G*T*T*T*T*G*T_C_G*T*T*T*T*G*T_C_G_T*T,;

(SEQ ID NO: 400)

T*C_G_T_C_G*T*T*T*T*G*T_C_G_T*T*T*T*G*T*C_G*T*T,;

(SEQ ID NO: 401)

T*C_G_T_C_G*T*T*T*T*G*T_C_G_T*T*T*T*G*T*C_G_T*T,;

(SEQ ID NO: 402)

T*C_G_T_C G*T*T*T*T*G*T_C_G_T*T*T*T*G*T_C_G*T*T,;

(SEQ ID NO: 403)

T*C_G_T_C_G*T*T*T*T*G*T_C_G_T*T*T*T*G*T_C_G_T*T,;

(SEQ ID NO: 404)

T*C_G_T_C_G_T*T*T*T*G*T*C_G*T*T*T*T*G*T*C_G*T*T,;

(SEQ ID NO: 405)

T*C_G_T_C_G_T*T*T*T*G*T*C_G*T*T*T*T*G*T*C_G_T*T,;

(SEQ ID NO: 406)

T*C_G_T_C_G_T*T*T*T*G*T*C_G*T*T*T*T*G*T_C_G*T*T,;

(SEQ ID NO: 407)

T*C_G_T_C_G_T*T*T*T*G*T*C_G*T*T*T*T*G*T_C_G_T*T,;

(SEQ ID NO: 408)

T*C_G_T_C_G_T*T*T*T*G*T*C_G_T*T*T*T*G*T*C_G*T*T,;

(SEQ ID NO: 409)

T*C_G_T_C_G_T*T*T*T*G*T*C_G_T*T*T*T*G*T*C_G_T*T,;

(SEQ ID NO: 410)

T*C_G_T_C_G_T*T*T*T*G*T*C_G_T*T*T*T*G*T_C_G*T*T,;

(SEQ ID NO: 411)

T*C_G_T_C_G_T*T*T*T*G*T*C_G_T*T*T*T*G*T_C_G_T*T,;

(SEQ ID NO: 412)

T*C_G_T_C_G_T*T*T*T*G*T_C_G*T*T*T*T*G*T*C_G*T*T,;

(SEQ ID NO: 413)

T*C_G_T_C_G_T*T*T*T*G*T_C_G*T*T*T*T*G*T*C_G_T*T,;

(SEQ ID NO: 414)

T*C_G_T_C_G_T*T*T*T*G*T_C_G*T*T*T*T*G*T_C_G*T*T,;

(SEQ ID NO: 415)

T*C_G_T_C_G_T*T*T*T*G*T_C_G*T*T*T*T*G*T_C_G_T*T,;

(SEQ ID NO: 416)

T*C_G_T_C_G_T*T*T*T*G*T_C_G_T*T*T*T*G*T*C_G*T*T,;

(SEQ ID NO: 417)

T*C_G_T_C_G_T*T*T*T*G*T_C_G_T*T*T*T*G*T*C_G_T*T,;

(SEQ ID NO: 418)

T*C_G_T_C_G_T*T*T*T*G*T_C_G_T*T*T*T*G*T_C_G*T*T,;
and

(SEQ ID NO: 419)

T*C_G_T_C_G_T*T*T*T*G*T_C_G_T*T*T*T*G*T_C_G_T*T,;

wherein * represents phosphorothioate and _{—} represents phosphodiester.

In one embodiment the immunostimulatory nucleic acid molecule is selected from the group consisting of:

(SEQ ID NO: 420)

T*C_G_T*C_G_T*T*T*T*G*T*C_G_T*T*T*T*G*T*C_G_T*T,;

(SEQ ID NO: 421)

T*C_G*T_C_G*T*T*T*T*G*T_C_G*T*T*T*T*G*T_C_G*T*T,;
and

(SEQ ID NO: 422)

T*C_G_T_C_G_T*T*T*T*G*T_C_G_T*T*T*T*G*T_C_G_T*T,;

wherein * represents phosphorothioate and _{—} represents phosphodiester.

In one embodiment the immunostimulatory nucleic acid molecule is selected from the group consisting of:

(SEQ ID NO: 423)

T*C*G*T*C*G*T*T*T_T_G*T*C*G*T*T*T_T_G*T*C*G*T*T,;

(SEQ ID NO: 424)

T*C*G*T*C*G*T*T*T*T_G_T*C*G*T*T*T*T_G_T*C*G*T*T,;
and

(SEQ ID NO: 425)

T*C*G*T*C*G*T*T*T_T_G_T*C*G*T*T*T_T_G_T*C*G*T*T,;

wherein * represents phosphorothioate and _{—} represents phosphodiester.

In one embodiment the immunostimulatory nucleic acid molecule is selected from the group consisting of: T*C_G*T_C_G*T*T*T_T_G*T_C_G*T*T*T_T_G*T_C_G*T*T, (SEQ ID NO: 426); T*C_G_T*C_G_T*T*T*T_G_T*C_G_T*T*T*T_G_T*C_G_T*T, (SEQ ID NO: 427); and T*C_G_T_C_G_T*T*T_T_G_T_C_G_T*T*T_T_G_T_C_G_T*T, (SEQ ID NO: 428); wherein * represents phosphorothioate and _{—} represents phosphodiester.

In one embodiment the at least one internal YG dinucleotide having a phosphodiester or phosphodiester-like internucleoside linkage is CG. In one embodiment the at least one internal YG dinucleotide having a phosphodiester or phosphodiester-like internucleoside linkage is TG.

In one embodiment the phosphodiester or phosphodiester-like internucleoside linkage is phosphodiester. In one embodiment the phosphodiester-like linkage is boranophosphonate or diastereomerically pure Rp phosphorothioate.

In one embodiment the stabilized internucleoside linkages are selected from the group consisting of: phosphorothioate, phosphorodithioate, methylphosphonate, methylphosphorothioate, and any combination thereof. In one embodiment the stabilized internucleoside linkages are phosphorothioate.

In one embodiment the immunostimulatory nucleic acid molecule is a type B immunostimulatory nucleic acid molecule. In one embodiment the immunostimulatory nucleic acid molecule is a type C immunostimulatory nucleic acid molecule.

In one embodiment the immunostimulatory nucleic acid molecule is 4-100 nucleotides long. In one embodiment the immunostimulatory nucleic acid molecule is 6-40 nucleotides long. In one embodiment the immunostimulatory nucleic acid molecule is 6-19 nucleotides long.

In one embodiment the immunostimulatory nucleic acid molecule is not an antisense oligonucleotide, triple-helix-forming oligonucleotide, or ribozyme.

In another aspect the invention provides an oligonucleotide which comprises

N₁-C_G-N₂-C_G-N₃

wherein N₁ and N₃ are each independently a nucleic acid sequence 1-20 nucleotides in length, wherein _{—} indicates an internal phosphodiester or phosphodiester-like internucleoside linkage, wherein N₂ is independently a nucleic acid sequence 0-20 nucleotides in length, and wherein G-N₂-C includes 1 or 2 stabilized linkages.

In another aspect the invention provides an oligonucleotide which comprises

N₁-C_G-N₂-C_G-N₃

wherein N₁ and N₃ are each independently a nucleic acid sequence 1-20 nucleotides in length, wherein _{—} indicates an internal phosphodiester or phosphodiester-like internucleoside linkage, wherein N₂ is independently a nucleic acid sequence 4-20 nucleotides in length, and wherein G-N₂-C includes at least 5 stabilized linkages.

In another aspect the invention provides an oligonucleotide which comprises

N₁-C_G-N₂-C_G-N₃

wherein N₁, N₂, and N₃ are each independently a nucleic acid sequence of 0-20 nucleotides in length and wherein _{—} indicates an internal phosphodiester or phosphodiester-like internucleoside linkage, wherein the oligonucleotide is not an antisense oligonucleotide, triple-helix-forming oligonucleotide, or ribozyme.

In another aspect the invention provides a an oligonucleotide which comprises

| | |
|---|---|
| X₁-N₁-(GTCGTT)ₙ-N₂-X₂ | (SEQ ID NOs: 429-433) |

wherein N₁ and N₂ are each independently a nucleic acid sequence of 0-20 nucleotides in length, wherein n=2 or n=4-6, wherein X₁ and X₂ are each independently a nucleic acid sequence having phosphorothioate internucleoside linkages of 3-10 nucleotides, wherein N₁-(GTCGTT)ₙ-N₂ includes at least one phosphodiester internucleoside linkage, and wherein 3′ and 5′ nucleotides of the oligonucleotide do not include a poly-G, poly-A, poly-T, or poly-C sequence.

The immunostimulatory nucleic acids can be double-stranded or single-stranded. Generally, double-stranded molecules are more stable in vivo, while single-stranded molecules have increased immune activity. Thus in some aspects of the invention it is preferred that the nucleic acid be single stranded and in other aspects it is preferred that the nucleic acid be double stranded.

For facilitating uptake into cells, the immunostimulatory nucleic acids are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

“Palindromic sequence” shall mean an inverted repeat (i.e., a sequence such as ABCDEE′D′C′B′A′ in which A and A′ are bases capable of forming the usual Watson-Crick base pairs). In vivo, such sequences may form double-stranded structures. In one embodiment, the immunostimulatory nucleic acid such as a CpG immunostimulatory nucleic acid contains a palindromic sequence. In one embodiment, a palindromic sequence contains a CpG which is preferably in the center of the palindrome. In another embodiment, the immunostimulatory nucleic acid such as a CpG immunostimulatory nucleic acid is free of a palindrome. For example, a CpG immunostimulatory nucleic acid that is free of a palindrome is one in which the CpG dinucleotide is not part of a palindrome. Such an oligonucleotide may include a palindrome in which the CpG dinucleotide is located outside of the palindrome.

In some embodiments of the invention, a non-CpG immunostimulatory nucleic acid is used. A non-CpG immunostimulatory nucleic acid is a nucleic acid which does not have a CpG motif in its sequence, regardless of whether the C in the dinucleotide is methylated or unmethylated. Non-CpG immunostimulatory nucleic acids may induce Th1 or Th2 immune responses, depending upon their sequence, their mode of delivery and the dose at which they are administered.

An important subset of non-CpG immunostimulatory nucleic acids are poly-G immunostimulatory nucleic acids. A variety of references, including Pisetsky and Reich, 1993 Mol. Biol. Reports, 18:217-221; Krieger and Herz, 1994, Ann. Rev. Biochem., 63:601-637; Macaya et al., 1993, PNAS, 90:3745-3749; Wyatt et al., 1994, PNAS, 91:1356-1360; Rando and Hogan, 1998, In Applied Antisense Oligonucleotide Technology, ed. Krieg and Stein, p. 335-352; and Kimura et al., 1994, J. Biochem. 116, 991-994 also describe the immunostimulatory properties of poly-G nucleic acids. In accordance with one aspect of the invention, poly-G-containing nucleotides are useful, inter alia, for treating and preventing bacterial, viral and fungal infections, and can thereby be used to minimize the impact of these infections on the treatment of cancer patients.

Poly-G nucleic acids preferably are nucleic acids having the following formulas:

5′ X₁X₂GGGX₃X₄ 3′

wherein X₁, X₂, X₃, and X₄ are nucleotides. In preferred embodiments at least one of X₃ and X₄ are a G. In other embodiments both of X₃ and X₄ are a G. In yet other embodiments the preferred formula is 5′ GGGNGGG 3′, or 5′ GGGNGGGNGGG 3′ (SEQ ID NO:434) wherein N represents between 0 and 20 nucleotides. In other embodiments the poly-G nucleic acid is free of unmethylated CG dinucleotides, such as, for example, the nucleic acids listed herein as SEQ ID NO: 95 through to SEQ ID NO: 133. In other embodiments the poly-G nucleic acid includes at least one unmethylated CG dinucleotide, such as, for example, the nucleic acids listed herein as SEQ ID NO: 46, SEQ ID NO: 47, SEQ ID NO: 58, and SEQ ID NO: 61.

Other non-CpG immunostimulatory nucleic acids are T-rich immunostimulatory nucleic acids or TG immunostimulatory nucleic acids. These nucleic acids are described in Published PCT Patent Application WO 01/22972 and related U.S. patent application Ser. No. 09/669,187 filed Sep. 25, 2000, the entire contents of which are incorporated herein by reference.

Immunostimulatory nucleic acids also include methylated CpG nucleic acids and nucleic acids having phosphate modified backbones, such as phosphorothioate backbones.

Methylated CpG nucleic acids are also immunostimulatory and useful for the purposes of the methods of the invention. A methylated CpG nucleic acid is a nucleic acid containing at least one CG dinucleotide in which the C of the CG is methylated and which does not include any unmethylated CG dinucleotides.

Exemplary immunostimulatory nucleic acid have the nucleotide sequences shown in Table 1. This list is not meant to be exhaustive, and one of ordinary skill will be able to arrive at other sequences for immunostimulatory nucleic acids based on the teachings provided herein.

TABLE 1

| | |
|---|---|
| GCTAGACGTTAGCGT; | (SEQ ID NO: 1) |
| GCTAGATGTTAGCGT; | (SEQ ID NO: 2) |
| GCTAGACGTTAGCGT; | (SEQ ID NO: 3) |
| GCTAGACGTTAGCGT; | (SEQ ID NO: 4) |
| GCATGACGTTGAGCT; | (SEQ ID NO: 5) |
| ATGGAAGGTCCAGCGTTCTC; | (SEQ ID NO: 6) |
| ATCGACTCTCGAGCGTTCTC; | (SEQ ID NO: 7) |
| ATCGACTCTCGAGCGTTCTC; | (SEQ ID NO: 8) |
| ATCGACTCTCGAGCGTTCTC; | (SEQ ID NO: 9) |
| ATGGAAGGTCCAACGTTCTC; | (SEQ ID NO: 10) |
| GAGAACGCTGGACCTTCCAT; | (SEQ ID NO: 11) |
| GAGAACGCTCGACCTTCCAT; | (SEQ ID NO: 12) |
| GAGAACGCTCGACCTTCGAT; | (SEQ ID NO: 13) |
| GAGAACGCTGGACCTTCCAT; | (SEQ ID NO: 14) |
| GAGAACGATGGACCTTCCAT; | (SEQ ID NO: 15) |
| GAGAACGCTCCAGCACTGAT; | (SEQ ID NO: 16) |
| TCCATGTCGGTCCTGATGCT; | (SEQ ID NO: 17) |
| TCCATGTCGGTCCTGATGCT; | (SEQ ID NO: 18) |
| TCCATGACGTTCCTGATGCT; | (SEQ ID NO: 19) |
| TCCATGTCGGTCCTGCTGAT; | (SEQ ID NO: 20) |
| TCAACGTT; | (SEQ ID NO: 21) |
| TCAGCGCT; | (SEQ ID NO: 22) |
| TCATCGAT; | (SEQ ID NO: 23) |
| TCTTCGAA; | (SEQ ID NO: 24) |
| CAACGTT; | (SEQ ID NO: 25) |
| CCAACGTT; | (SEQ ID NO: 26) |
| AACGTTCT; | (SEQ ID NO: 27) |
| TCAACGTC; | (SEQ ID NO: 28) |
| ATGGACTCTCCAGCGTTCTC; | (SEQ ID NO: 29) |
| ATGGAAGGTCCAACGTTCTC; | (SEQ ID NO: 30) |
| ATCGACTCTCGAGCGTTCTC; | (SEQ ID NO: 31) |
| ATGGAGGCTCCATCGTTCTC; | (SEQ ID NO: 32) |
| ATCGACTCTCGAGCGTTCTC; | (SEQ ID NO: 33) |
| ATCGACTCTCGAGCGTTCTC; | (SEQ ID NO: 34) |
| TCCATGTCGGTCCTGATGCT; | (SEQ ID NO: 35) |
| TCCATGCCGGTCCTGATGCT; | (SEQ ID NO: 36) |
| TCCATGGCGGTCCTGATGCT; | (SEQ ID NO: 37) |
| TCCATGACGGTCCTGATGCT; | (SEQ ID NO: 38) |
| TCCATGTCGATCCTGATGCT; | (SEQ ID NO: 39) |
| TCCATGTCGCTCCTGATGCT; | (SEQ ID NO: 40) |
| TCCATGTCGTCCCTGATGCT; | (SEQ ID NO: 41) |
| TCCATGACGTGCCTGATGCT; | (SEQ ID NO: 42) |
| TCCATAACGTTCCTGATGCT; | (SEQ ID NO: 43) |
| TCCATGACGTCCCTGATGCT; | (SEQ ID NO: 44) |
| TCCATCACGTGCCTGATGCT; | (SEQ ID NO: 45) |
| GGGGTCAACGTTGACGGGG; | (SEQ ID NO: 46) |
| GGGGTCAGTCGTGACGGGG; | (SEQ ID NO: 47) |
| GCTAGACGTTAGTGT; | (SEQ ID NO: 48) |
| TCCATGTCGTTCCTGATGCT; | (SEQ ID NO: 49) |
| ACCATGGACGATCTGTTTCCCCTC; | (SEQ ID NO: 50) |
| TCTCCCAGCGTGCGCCAT; | (SEQ ID NO: 51) |
| ACCATGGACGAACTGTTTCCCCTC; | (SEQ ID NO: 52) |
| ACCATGGACGAGCTGTTTCCCCTC; | (SEQ ID NO: 53) |
| ACCATGGACGACCTGTTTCCCCTC; | (SEQ ID NO: 54) |
| ACCATGGACGTACTGTTTCCCCTC; | (SEQ ID NO: 55) |
| ACCATGGACGGTCTGTTTCCCCTC; | (SEQ ID NO: 56) |
| ACCATGGACGTTCTGTTTCCCCTC; | (SEQ ID NO: 57) |
| CACGTTGAGGGGCAT; | (SEQ ID NO: 58) |
| TCAGCGTGCGCC; | (SEQ ID NO: 59) |
| ATGACGTTCCTGACGTT; | (SEQ ID NO: 60) |
| TCTCCCAGCGGGCGCAT; | (SEQ ID NO: 61) |
| TCCATGTCGTTCCTGTCGTT; | (SEQ ID NO: 62) |
| TCCATAGCGTTCCTAGCGTT; | (SEQ ID NO: 63) |
| TCGTCGCTGTCTCCCCTTCTT; | (SEQ ID NO: 64) |
| TCCTGACGTTCCTGACGTT; | (SEQ ID NO: 65) |
| TCCTGTCGTTCCTGTCGTT; | (SEQ ID NO: 66) |
| TCCATGTCGTTTTTGTCGTT; | (SEQ ID NO: 67) |
| TCCTGTCGTTCCTTGTCGTT; | (SEQ ID NO: 68) |
| TCCTTGTCGTTCCTGTCGTT; | (SEQ ID NO: 69) |
| TCCTGTCGTTTTTTGTCGTT; | (SEQ ID NO: 70) |
| TCGTCGCTGTCTGCCCTTCTT; | (SEQ ID NO: 71) |
| TCGTCGCTGTTGTCGTTTCTT; | (SEQ ID NO: 72) |
| TCCATGCGTGCGTGCGTTTT; | (SEQ ID NO: 73) |
| TCCATGCGTTGCGTTGCGTT; | (SEQ ID NO: 74) |
| TCCACGACGTTTTCGACGTT; | (SEQ ID NO: 75) |
| TCGTCGTTGTCGTTGTCGTT; | (SEQ ID NO: 76) |
| TCGTCGTTTTGTCGTTTTGTCGTT; | (SEQ ID NO: 77) |
| TCGTCGTTGTCGTTTTGTCGTT; | (SEQ ID NO: 78) |
| GCGTGCGTTGTCGTTGTCGTT; | (SEQ ID NO: 79) |
| TGTCGTTTGTCGTTTGTCGTT; | (SEQ ID NO: 80) |
| TGTCGTTGTCGTTGTCGTTGTCGTT; | (SEQ ID NO: 81) |
| TGTCGTTGTCGTTGTCGTT; | (SEQ ID NO: 82) |
| TCGTCGTCGTCGTT; | (SEQ ID NO: 83) |
| TGTCGTTGTCGTT; | (SEQ ID NO: 84) |
| TCCATAGCGTTCCTAGCGTT; | (SEQ ID NO: 85) |
| TCCATGACGTTCCTGACGTT; | (SEQ ID NO: 86) |
| GTCGYT; | (SEQ ID NO: 87) |
| TGTCGYT; | (SEQ ID NO: 88) |
| AGCTATGACGTTCCAAGG; | (SEQ ID NO: 89) |
| TCCATGACGTTCCTGACGTT; | (SEQ ID NO: 90) |
| ATCGACTCTCGAACGTTCTC; | (SEQ ID NO: 91) |
| TCCATGTCGGTCCTGACGCA; | (SEQ ID NO: 92) |
| TCTTCGAT; | (SEQ ID NO: 93) |
| ATAGGAGGTCCAACGTTCTC; | (SEQ ID NO: 94) |
| GCTAGAGGGGAGGGT; | (SEQ ID NO: 95) |
| GCTAGATGTTAGGGG; | (SEQ ID NO: 96) |
| GCTAGAGGGGAGGGT; | (SEQ ID NO: 97) |
| GCTAGAGGGGAGGGT; | (SEQ ID NO: 98) |
| GCATGAGGGGGAGCT; | (SEQ ID NO: 99) |
| ATGGAAGGTCCAGGGGGCTC; | (SEQ ID NO: 100) |
| ATGGACTCTGGAGGGGGCTC; | (SEQ ID NO: 101) |
| ATGGACTCTGGAGGGGGCTC; | (SEQ ID NO: 102) |
| ATGGACTCTGGAGGGGGCTC; | (SEQ ID NO: 103) |
| ATGGAAGGTCCAAGGGGCTC; | (SEQ ID NO: 104) |
| GAGAAGGGGGGACCTTCCAT; | (SEQ ID NO: 105) |
| GAGAAGGGGGGACCTTCCAT; | (SEQ ID NO: 106) |
| GAGAAGGGGGGACCTTGGAT; | (SEQ ID NO: 107) |
| GAGAAGGGGGGACCTTCCAT; | (SEQ ID NO: 108) |
| GAGAAGGGGGGACCTTCCAT; | (SEQ ID NO: 109) |
| GAGAAGGGGCCAGCACTGAT; | (SEQ ID NO: 110) |
| TCCATGTGGGGCCTGATGCT; | (SEQ ID NO: 111) |
| TCCATGTGGGGCCTGATGCT; | (SEQ ID NO: 112) |
| TCCATGAGGGGCCTGATGCT; | (SEQ ID NO: 113) |
| TCCATGTGGGGCCTGCTGAT; | (SEQ ID NO: 114) |
| ATGGACTCTCCGGGGTTCTC; | (SEQ ID NO: 115) |
| ATGGAAGGTCCGGGGTTCTC; | (SEQ ID NO: 116) |
| ATGGACTCTGGAGGGGTCTC; | (SEQ ID NO: 117) |
| ATGGAGGCTCCATGGGGCTC; | (SEQ ID NO: 118) |
| ATGGACTCTGGGGGGTTCTC; | (SEQ ID NO: 119) |
| ATGGACTCTGGGGGGTTCTC; | (SEQ ID NO: 120) |
| TCCATGTGGGTGGGGATGCT; | (SEQ ID NO: 121) |
| TCCATGCGGGTGGGGATGCT; | (SEQ ID NO: 122) |
| TCCATGGGGGTCCTGATGCT; | (SEQ ID NO: 123) |
| TCCATGGGGGTCCTGATGCT; | (SEQ ID NO: 124) |
| TCCATGTGGGGCCTGATGCT; | (SEQ ID NO: 125) |
| TCCATGTGGGGCCTGATGCT; | (SEQ ID NO: 126) |
| TCCATGGGGTCCCTGATGCT; | (SEQ ID NO: 127) |
| TCCATGGGGTGCCTGATGCT; | (SEQ ID NO: 128) |
| TCCATGGGGTTCCTGATGCT; | (SEQ ID NO: 129) |
| TCCATGGGGTCCCTGATGCT; | (SEQ ID NO: 130) |
| TCCATCGGGGGCCTGATGCT; | (SEQ ID NO: 131) |
| GCTAGAGGGAGTGT; | (SEQ ID NO: 132) |
| GGGGGGGGGGGGGGGGGGGG; | (SEQ ID NO: 133) |
| ACTGACAGACTGACAGACTGA; | (SEQ ID NO: 134) |
| AGTGACAGACAGACACACTGA; | (SEQ ID NO: 135) |
| ACTGACAGACTGATAGACCCA; | (SEQ ID NO: 136) |
| AGTGAGAGACTGCAAGACTGA; | (SEQ ID NO: 137) |
| AATGCCAGTCCGACAGGCTGA; | (SEQ ID NO: 138) |
| CCAGAACAGAAGCAATGGATG; | (SEQ ID NO: 139) |
| CCTGAACAGAAGCCATGGATG; | (SEQ ID NO: 140) |
| GCAGAACAGAAGACATGGATG; | (SEQ ID NO: 141) |
| CCACAACACAAGCAATGGATA; | (SEQ ID NO: 142) |
| AAGCTAGCCAGCTAGCTAGCA; | (SEQ ID NO: 143) |
| CAGCTAGCCACCTAGCTAGCA; | (SEQ ID NO: 144) |
| AAGCTAGGCAGCTAACTAGCA; | (SEQ ID NO: 145) |
| GAGCTAGCAAGCTAGCTAGGA; | (SEQ ID NO: 146) |

For use in the instant invention, the immunostimulatory nucleic acids may be synthesized de novo using any of a number of procedures well known in the art. Such compounds are referred to as “synthetic” nucleic acids. For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid Res. 14:5399-5407, 1986,; Garegg et al., Tet. Let. 27:4055-4058, 1986, Gaffney et al., Tet. Let. 29:2619-2622, 1988). These chemistries can be performed by a variety of automated oligonucleotide synthesizers available in the market. These nucleic acids are referred to as synthetic nucleic acids. Alternatively, immunostimulatory nucleic acids can be produced on a large scale in plasmids, (see Sambrook, T., et al., “Molecular Cloning: A Laboratory Manual”, Cold Spring Harbor laboratory Press, New York, 1989) and separated into smaller pieces or administered whole. Nucleic acids can be prepared from existing nucleic acid sequences (e.g., genomic or cDNA) using known techniques, such as those employing restriction enzymes, exonucleases or endonucleases. Nucleic acids prepared in this manner are referred to as isolated nucleic acids. The term “immunostimulatory nucleic acid” encompasses both synthetic immunostimulatory nucleic acids and those isolated from natural sources.

For use in vivo, nucleic acids are preferably relatively resistant to degradation (e.g., are stabilized). A “stabilized nucleic acid molecule” shall mean a nucleic acid molecule that is relatively resistant to in vivo degradation (e.g. via an exo- or endo-nuclease). Stabilization can be a function of length or secondary structure. Immunostimulatory nucleic acids that are tens to hundreds of kbs long are relatively resistant to in vivo degradation. For shorter immunostimulatory nucleic acids, secondary structure can stabilize and increase their effect. For example, if the 3′ end of a nucleic acid has self-complementarity to an upstream region, so that it can fold back and form a sort of stem loop structure, then the nucleic acid becomes stabilized and therefore exhibits more biological in vivo activity.

Alternatively, nucleic acid stabilization can be accomplished via backbone modifications. Preferred stabilized nucleic acids of the instant invention have a modified backbone. It has been demonstrated that modification of the nucleic acid backbone provides enhanced activity of the immunostimulatory nucleic acids when administered in vivo. One type of modified backbone is a phosphate backbone modification. Immunostimulatory nucleic acids, including at least two phosphorothioate linkages at the 5′ end of the oligonucleotide and multiple phosphorothioate linkages at the 3′ end, preferably 5, can in some circumstances provide maximal activity and protect the nucleic acid from degradation by intracellular exo- and endo-nucleases. Other phosphate modified nucleic acids include phosphodiester modified nucleic acids, combinations of phosphodiester and phosphorothioate nucleic acids, methylphosphonate, methylphosphorothioate, phosphorodithioate, and combinations thereof. Each of these combinations in CpG nucleic acids and their particular effects on immune cells is discussed in more detail in issued U.S. Pat. Nos. 6,194,388; 6,207,646, and 6,239,116, the entire contents of which are hereby incorporated by reference. Although not intending to be bound by any particular theory, it is believed that these phosphate modified nucleic acids may show more stimulatory activity due to enhanced nuclease resistance, increased cellular uptake, increased protein binding, and/or altered intracellular localization.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863. Alkylphosphotriesters, in which the charged oxygen moiety is alkylated as described in U.S. Pat. No. 5,023,243 and European Patent No. 092,574, can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

Both phosphorothioate and phosphodiester nucleic acids containing immunostimulatory motifs are active in immune cells. However, based on the concentration needed to induce immunostimulatory nucleic acid specific effects, the nuclease resistant phosphorothioate backbone immunostimulatory nucleic acids are more potent than phosphodiester backbone immunostimulatory nucleic acids. For example, 2 μg/ml of the phosphorothioate has been shown to effect the same immune stimulation as a 90 μg/ml of the phosphodiester.

Another type of modified backbone, useful according to the invention, is a peptide nucleic acid. The backbone is composed of aminoethylglycine and supports bases which provide the DNA character. The backbone does not include any phosphate and thus may optionally have no net charge. The lack of charge allows for stronger DNA-DNA binding because the charge repulsion between the two strands does not exist. Additionally, because the backbone has an extra methylene group, the oligonucleotides are enzyme/protease resistant. Peptide nucleic acids can be purchased from various commercial sources, e.g., Perkin Elmer, or synthesized de novo.

Another class of backbone modifications include 2′-O-methylribonucleosides (2′-Ome). These types of substitutions are described extensively in the prior art and in particular with respect to their immunostimulating properties in Zhao et al., Bioorganic and Medicinal Chemistry Letters, 1999, 9:24:3453. Zhao et al. describes methods of preparing 2′-Ome modifications to nucleic acids.

The nucleic acid molecules of the invention may include naturally-occurring or synthetic purine or pyrimidine heterocyclic bases as well as modified backbones. Purine or pyrimidine heterocyclic bases include, but are not limited to, adenine, guanine, cytosine, thymidine, uracil, and inosine. Other representative heterocyclic bases are disclosed in U.S. Pat. No. 3,687,808, issued to Merigan, et al. The terms “purines” or “pyrimidines” or “bases” are used herein to refer to both naturally-occurring or synthetic purines, pyrimidines or bases.

Other stabilized nucleic acids include non-ionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The immunostimulatory nucleic acids having backbone modifications useful according to the invention in some embodiments are S- or R-chiral immunostimulatory nucleic acids. An “S chiral immunostimulatory nucleic acid” as used herein is an immunostimulatory nucleic acid wherein at least two nucleotides have a backbone modification forming a chiral center and wherein at least 75% of the chiral centers have S chirality. An “R chiral immunostimulatory nucleic acid” as used herein is an immunostimulatory nucleic acid wherein at least two nucleotides have a backbone modification forming a chiral center and wherein at least 75% of the chiral centers have R chirality. The backbone modification may be any type of modification that forms a chiral center. The modifications include but are not limited to phosphorothioate, methylphosphonate, methylphosphorothioate, phosphorodithioate, 2′-Ome and combinations thereof.

The chiral immunostimulatory nucleic acids must have at least two nucleotides within the nucleic acid that have a backbone modification. All or less than all of the nucleotides in the nucleic acid, however, may have a modified backbone. Of the nucleotides having a modified backbone (referred to as chiral centers), at least 75% of the have a single chirality, S or R. Thus, less than all of the chiral centers may have S or R chirality as long as at least 75% of the chiral centers have S or R chirality. In some embodiments at least 80,%, 85%, 90%, 95%, or 100% of the chiral centers have S or R chirality. In other embodiments at least 80%, 85%, 90%, 95%, or 100% of the nucleotides have backbone modifications.

The S- and R-chiral immunostimulatory nucleic acids may be prepared by any method known in the art for producing chirally pure oligonucleotides. Stec et al teach methods for producing stereopure phosphorothioate oligodeoxynucleotides using an oxathiaphospholane. (Stec, W. J., et al., 1995, J. Am. Chem. Soc., 117:12019). Other methods for making chirally pure oligonucleotides have been described by companies such as ISIS Pharmaceuticals. US patents which disclose methods for generating stereopure oligonucleotides include U.S. Pat. Nos. 5,883,237, 5,837,856, 5,599,797, 5,512,668, 5,856,465, 5,359,052, 5,506,212, 5,521,302 and 5,212,295, each of which is hereby incorporated by reference in its entirety.

As used herein, administration of an immunostimulatory nucleic acid is intended to embrace the administration of one or more immunostimulatory nucleic acids which may or may not differ in terms of their profile, sequence, backbone modifications and biological effect. As an example, CpG nucleic acids and poly-G nucleic acids may be administered to a single subject. In another example, a plurality of CpG nucleic acids which differ in nucleotide sequence may also be administered to a subject.

The formulations of the invention are oil-in-water emulsions. As used herein the term oil-1n-water emulsion refers to a fluid composed of a heterogeneous mixture of minute drops of oil suspended in water. Oil-in-water emulsions are well known in the art. One preferred oil-in-water emulsion for non-human subjects is sold under the trademark name EMULSIGEN™ (sold by MPV Laboratories, Nebraska, U.S.A).

The term “effective amount” of an immunostimulatory nucleic acid refers to the amount necessary or sufficient to realize a desired biologic effect. For example, an effective amount of an immunostimulatory nucleic acid could be that amount necessary to cause activation of the immune system, resulting potentially in the development of an antigen specific immune response. According to some aspects of the invention, an effective amount is that amount of an immunostimulatory nucleic acid in an oil-in-water emulsion which results in a synergistic response to the cancer or infectious agent, either in the prevention or the treatment of the cancer or infectious disease. A synergistic amount is that amount which produces a response that is greater than the sum of the individual effects of the agents. For example, a synergistic combination of an immunostimulatory nucleic acid and an oil-in-water emulsion provides a biological effect that is greater than the combined biological effect which could have been achieved using each of the components separately. The biological effect may be the amelioration and or absolute elimination of symptoms resulting from the cancer or infectious disease. In another embodiment, the biological effect is the complete abrogation of the cancer or infectious disease, as evidenced for example, by the absence of a tumor or a biopsy or blood smear that is free of cancer cells.

The effective amount of immunostimulatory nucleic acid necessary to synergize with an oil-in-water emulsion in the treatment of a cancer or infectious disease or in the reduction of the risk of developing a cancer or infectious disease may vary depending upon the sequence the backbone constituents of the nucleic acid, and the mode of delivery of the nucleic acid. The effective amount for any particular application can also vary depending on such factors as the disease being treated, the particular immunostimulatory nucleic acid being administered (e.g. the nature, number or location of immunostimulatory motifs in the nucleic acid), the size of the subject, and/or the severity of the disease or condition. One of ordinary skill in the art can empirically determine the effective amount of a particular immunostimulatory nucleic acid and oil-in-water emulsion combination without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

In some embodiments, the immunostimulatory nucleic acids are administered in an effective amount to stimulate or induce a Th1 immune response, or a Th2 immune response, or a general immune response. An effective amount to stimulate a Th1 immune response may be defined as that amount which stimulates the production of one or more Th1-type cytokines such as interleukin 2 (IL-2), IL-12, tumor necrosis factor (TNFα) and interferon gamma (IFN-γ), and/or production of one or more Th1-type antibodies. An effective amount to stimulate a Th2 immune response, on the other hand, may be defined as that amount which stimulates the production of one or more Th2-type cytokines such as IL-4, IL-5, IL-6, IL-9, IL-10 and IL-13, and/or the production of one or more Th2-type antibodies.

In some embodiments of the invention, the immunostimulatory nucleic acid is administered in an effective amount for preventing bacterial, viral, fungal or parasitic infection.

In some instances, a sub-optimal or sub-therapeutic dosage of the antigen is used in a prophylactic or therapeutic vaccine to administer to a subject having, or at risk of developing, cancer or an infectious disease. As an example, it has been discovered according to the invention, that when the antigen is used together with the immunostimulatory nucleic acid, the antigen can be administered in a sub-therapeutic dose and still produce a desirable therapeutic result. A “sub-therapeutic dose” as used herein refers to a dosage that is less than that dosage which would produce a therapeutic result in the subject if administered in the absence of the other agent. Thus, the sub-therapeutic dose of an antigen is one which, alone or in combination with an adjuvant such as alum, would not produce the desired therapeutic result in the subject in the absence of the administration of the immunostimulatory nucleic acid. Therapeutic doses of antigens are well known in the field of vaccination. These dosages have been extensively described in references relied upon by the medical profession as guidance for vaccination. Therapeutic dosages of immunostimulatory nucleic acids have also been described in the art and methods for identifying therapeutic dosages in subjects are described in more detail herein.

The effective amount of immunostimulatory nucleic acid can be determined using in vitro stimulation assays. The stimulation index of the immunostimulatory nucleic acid can be compared to that of previously tested immunostimulatory acids. The stimulation index can be used to determine an effective amount of the particular oligonucleotide for the particular subject, and the dosage can be adjusted upwards or downwards to achieve the desired levels in the subject.

Therapeutically effective amounts can also be determined in animal studies. For instance, the effective amount of an immunostimulatory nucleic acid in an oil-in-water emulsion to induce a synergistic response when administered topically can be assessed using in vivo assays of tumor regression and/or prevention of tumor formation. Relevant animal models include assays in which malignant cells are injected into the animal subjects, usually in a defined topical site. Generally, a range of doses of an immunostimulatory nucleic acid in an emulsion is administered topically to the animal. Inhibition of the growth of a tumor following the injection of the malignant cells is indicative of the ability to reduce the risk of developing a cancer. Inhibition of further growth (or reduction in size) of a pre-existing tumor is indicative of the ability to treat the cancer. Mice, which have been modified to have human immune system elements, can be used as recipients of human cancer cell lines to determine the effective amount of the synergistic combination.

An effective dose can also be determined from human data for immunostimulatory nucleic acids which have been tested in humans (human clinical trials have been initiated) and for compounds that are known to exhibit similar pharmacological activities, such as other adjuvants, e.g., LT and other antigens for vaccination purposes.

The applied dose of the emulsion/nucleic acid formulation can be adjusted based on the relative bioavailability and potency of the administered compounds. Adjusting the dose to achieve maximal efficacy based on the methods described above and other methods are well within the capabilities of the ordinarily skilled artisan.

Subject doses of the compounds described herein typically range from about 0.1 μg to 1,000 mg, more typically from about 10 μg/day to 100 mg, and most typically from about 100 μg to 10 mg. Stated in terms of subject body weight, typical dosages range from about 0.002 μg to 200 mg/kg/day, more typically from about 0.2 μg/kg/day to 2 mg/kg/day, and most typically from about 2 μg/kg/day to 0.2 mg/kg/day.

In other embodiments of the invention, the emulsion/nucleic acid formulation is administered on a routine schedule. A “routine schedule” as used herein, refers to a predetermined designated period of time. The routine schedule may encompass periods of time which are identical or which differ in length, as long as the schedule is predetermined. For instance, the routine schedule may involve administration on a daily basis, multiple times per day, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the on a daily basis for the first week, followed by a monthly basis for several months, and then every three months after that. Any particular combination would be covered by the routine schedule as long as it is determined ahead of time that the appropriate schedule involves administration on a certain day.

The immunostimulatory nucleic acids may be delivered to the subject in the form of a plasmid vector. In some embodiments, one plasmid vector could include both the immunostimulatory nucleic acid and a nucleic acid encoding an antigen. In other embodiments, separate plasmids could be used. In yet other embodiments, no plasmids could be used.

The emulsion/nucleic acid formulation may be administered alone (e.g. in saline or buffer) or using any delivery vectors known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The emulsion/nucleic acid and formulation may be combined with additional therapeutic agents such as cytokines to enhance immune responses even further. The emulsion/nucleic formulation and other therapeutic agent may be administered simultaneously or sequentially. When the other therapeutic agents are administered simultaneously they can be administered in the same or separate formulations, in the same or different routes, but are at least administered at the same time. The administration of the other therapeutic agents and the emulsion/nucleic acid formulation may also be temporally separated, meaning that the therapeutic agents are administered at a different time, either before or after, the administration of the emulsion/nucleic acid formulation. The separation in time between the administration of these compounds may be a matter of minutes or it may be longer. Other therapeutic agents include but are not limited to immunotherapeutic antibodies, other immune modulators, antigens, anti-microbial agents, cancer medicaments, etc.

Immune responses can also be induced or augmented by the co-administration or co-linear expression of cytokines or co-stimulatory molecules with the emulsion/nucleic acid formulations. The cytokines may be administered directly with emulsion/nucleic acid formulation or may be administered in the form of a nucleic acid vector that encodes the cytokine, such that the cytokine can be expressed in vivo. In one embodiment, the cytokine is administered in the form of a plasmid expression vector. The term “cytokine” is used as a generic name for a diverse group of soluble proteins and peptides which act as humoral regulators at nano- to pico-molar concentrations and which, either under normal or pathological conditions, modulate the functional activities of individual cells and tissues. These proteins also mediate interactions between cells directly and regulate processes taking place in the extracellular environment. Cytokines also are central in directing the T cell response. Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), interferon-γ (IFN-γ), IFN-α, tumor necrosis factor (TNF), TGF-β, FLT-3 ligand, and CD40 ligand. In some embodiments, the cytokine is a Th1 cytokine. In still other embodiments, the cytokine is a Th2 cytokine. In other embodiments a cytokine is not administered in combination with the emulsion/nucleic acid formulation.

Other therapeutic agents that can be administered with the nucleic acids of the invention are mucosal adjuvants. Mucosal adjuvants are most preferably used when the nucleic acids are administered directly to a mucosal surface. The mucosal adjuvants useful according to the invention are non-oligonucleotide mucosal adjuvants. A “non-oligonucleotide mucosal adjuvant” as used herein is an adjuvant other than an immunostimulatory oligonucleotide that is capable of inducing a mucosal immune response in a subject when administered to a mucosal surface in conjunction with an antigen. Mucosal adjuvants include but are not limited to bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); and CTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler B D, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi, outer membrane protine of Neisseria meningitidis) (Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worster, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

In other aspects, the invention relates to kits. One kit of the invention includes a container housing an immunostimulatory nucleic acid and a container housing an oil-in-water emulsion and instructions for timing of administration of the immunostimulatory nucleic acid and the oil-in-water emulsion. Another kit of the invention includes a container housing an immunostimulatory nucleic acid in an oil-in-water emulsion and instructions for timing of administration. Optionally the kit may also include an antigen, housed in a separate container or formulated with the immunostimulatory nucleic acid or the oil-in-water emulsion. Optionally the antigen may be in a sustained release device. A sustained release vehicle is used herein in accordance with its prior art meaning of any device which slowly releases the antigen. The kit preferably contains or is suited to topical administration. For example, the delivery device may be appropriate for ocular delivery (such as an ocular ointment), for oral delivery (such as an oral gel), for vaginal or rectal delivery (such as a vaginal or rectal cream), and the like.

The formulations such as the oil-in-water-emulsion are housed in at least one container. The container may be a single container housing all of the emulsion or it may be multiple containers or chambers housing individual dosages of the emulsion, such as a blister pack. The kit also has instructions for timing of administration of the therapeutic formulation. The instructions would direct the subject having cancer or at risk of cancer to take the therapeutic formulation at the appropriate time. For instance, the appropriate time for delivery of the medicament may be as the symptoms occur. Alternatively, the appropriate time for administration of the medicament may be on a routine schedule such as monthly or yearly.

The emulsion/nucleic acid formulation may be administered by any ordinary route for administering medications although a topical route of administration is preferred. Depending upon the type of disorder to be treated, the formulations may be inhaled, ingested or administered to any external surface such as the skin or an mucosal (preferably external mucosal) surface. Inhalation will deliver the compounds to the nasal cavity and ingestion will deliver the compounds to at least the oral cavity. Preferred routes of administration include but are not limited to oral, intranasal, intratracheal, inhalation, ocular, vaginal, rectal, and dermal.

For use in therapy, an effective amount of the emulsion/nucleic acid formulation can be administered to a subject by any mode that delivers the nucleic acid to a skin or mucosal surface. “Administering” the pharmaceutical composition of the present invention may be accomplished by any means known to the skilled artisan.

It is important to note that in preferred embodiments, the compositions of the invention are formulated so as to adopt a cream-like consistency. Accordingly, they are provided to a subject in a cream or ointment or gel rather than a liquid solution, or a dried powder.

The formulations will be provided in different vessels, vehicles or formulations depending upon the disorder and mode of administration. For example, and as described in greater detail herein, for oral application, the compounds can be administered as sublingual tablets (provided these are capable of containing the oil-in-water emulsions), toothpastes, gels, creams, films, etc.; for ocular application, eye ointments, eye gels; for topical application, as lotions, ointments, gels, creams, etc.; for vaginal or rectal application, as an ointment, a suppository, a mucoadhesive formulation, etc.

Importantly, the carrier must be suitable for the body tissue or surface that it contacts. As will be known to those of ordinary skill in the art, carriers suitable for ocular administration are required to induce minimal, and preferably, no irritation to the eye. Ocular or ophthalmic formulations are known in the pharmaceutical arts and one of ordinary skill can consult Remington's Pharmaceuticals for guidance as to the composition of such carriers.

The compositions intended for ocular administration must be compatible with the eye environment, at least in terms of pH, and salt composition and concentration. These compositions should not irritate the eye. Compositions can be administered to the eye in various physical forms including but not limited to an ophthalmic ointment or gel, and the like.

For ocular use, formulations that do not contain preservatives, such as ophthalmic preservatives, tend to have a shorter shelf life and thus are generally prepared in smaller volumes. Thus, in some important embodiments, the compositions are provided in pouches (and the like) that contain at a maximum, volumes on the order of 0.5 ml to 5.0 ml. These latter embodiments correspond to single use, or single week units, and optionally they do not contain ophthalmic preservatives. A plurality of such small volume housing can be provided in a kit, that can optionally comprise an outer housing such as a box or bag, or a backing such as a cardboard or plastic backing. The kit can contain instructions for use of the composition, as outlined herein.

The compositions can also be provided on the surface of films. In some important embodiments, the compositions are formulated as ocular gels or ointments, such as those known in the art.

Compositions intended for ocular administration may contain other agents that have been described for ocular ointments, gels, etc. or that are known to be present in tears. An example is lysozyme which is known to be present in tears.

In some embodiments involving ocular administration, the composition may be treated in order to eliminate color (thus rendering the solution clear and colorless). Alternatively, it may be desirable to add or change the color of the composition, particularly if color is used to confirm delivery of the composition to the eye.

In some embodiments, the ocular compositions do not contain preservatives, and rather are sterile filtered (e.g., through a 0.22 μm filter) or heated, and packaged as single use amounts. Thus, in some instances, the compositions are prepared and/or packaged in unit of use amounts. A unit of use amount may be that amount that is required for one administration, or administrations for one day, one week, one month, or longer. Preferably, a unit of unit amount will be that amount required for either one administration or for at most several days (but less than a week) of administration. Unit of use packaging is useful for preventing contamination of solutions, as it reduces the number of times an individual must contact the solution.

Ophthalmic preservatives are known in the art. Generally, such preservatives are antibiotics, as bacterial infections are one of the most common side effects of administering agents to the eye. Examples of ophthalmic preservatives include organic mercurials (e.g., phenylmercuric nitrate, phenylmercuric acetate, phenylmercuric borate, Thimerosal (Merthiolate®, Lilly)); quaternary ammonium compounds (e.g., benzalkonium chloride), benzethonium chloride, cetyl pyridinium chloride, polyquaternium-1 (POLYQUAD)); parahydroxybenzoic acid esters; and substituted alcohols and phenols (e.g., chlorobutanol, chlorobutanol/phenylethyl alcohol). Other suitable preservatives include methyl paraben and propyl paraben.

Ophthalmic formulations can further include isotonicity agents, buffering agents, preservatives (as discussed above), diluents, stabilizers, chelating agents, thickeners, etc. Examples of isotonicity agents include sodium chloride, boric acid, sodium citrate, etc. Examples of buffering agents include borate buffer, phosphate buffer, etc. Examples of diluents include distilled or sterilized water or physiological saline (for aqueous formulations), and vegetable oils, liquid paraffin, mineral oil, propylene glycol, and p-octyldodecanol (for non-aqueous formulations). Examples of stabilizers include sodium sulfite and propylene glycol. An example of a suitable chelating agent is sodium EDTA. Examples of thickeners include glycerol, carboxymethylcellulose, and carboxyvinyl polymer.

Other components that can be included in ophthalmic formulations include sorbic acid, sodium dihydrogen phosphate, sodium borate, sodium hydroxide, potassium chloride, calcium chloride, glycerin, lysozyme, etc.

The compositions can similarly be administered to subjects in a variety of physical forms suitable for oral or buccal administration. The terms “oral” and “buccal” are used interchangeably herein to indicate the oral cavity, encompassing the lips, teeth, mouth, tongue, palate, and throat region. The compositions intended for oral or buccal administration must be compatible with the environment of the oral cavity. The requirements for oral or buccal delivery formulations are generally less strict than those for ocular delivery formulations. However, taste and odor considerations are important in oral or buccal formulations and are most probably less important for ocular formulations.

In preferred embodiments, compositions are delivered to and remain in the oral cavity, regardless of their physical form. Thus, it is preferable that the compositions are provided in forms such as lozenges, gums, and sublingual tablets (provided they are capable of containing the oil-in-water emulsion); oral gels, toothpastes, mucoadhesive patches (onto which the oil-in-water emulsion is coated), and the like, that remain in the oral cavity and are not ingested into the gastrointestinal tract.

When delivered orally, the compositions contact the oral mucosa including the sublingual mucosa. “Mucosa” refers to a mucous membrane. “Oral mucosa” as used herein refers to the mucosa of the mouth and upper throat region. “Sublingual” refers to the area of the oral cavity below the tongue.

For oral administration, the compounds (i.e., immunostimulatory nucleic acids, therapeutic formulations, and the other therapeutic agents) may be formulated readily by combining the active compound(s) with pharmaceutically acceptable carriers well known in the art. Such carriers enable the compounds of the invention to be formulated as capsules, gels, syrups, slurries, suspensions and the like, for oral delivery by a subject to be treated. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

The compositions can also be formulated as oral gels or creams. As an example, the compositions may be administered in a mucosally adherent, water soluble gel. The compositions can also be formulated as toothpastes.

Where necessary, delivery formulations may comprise flavoring, coloring and/or scenting agents. Flavoring, coloring and/or scenting agents help to improve user acceptance of the composition.

Flavoring agents are agents that provide a taste to an otherwise tasteless formulation, agents that enhance a pre-existing but weak taste, or agents that mask or change a pre-existing and unpalatable taste to one that is more palatable. Flavoring agents are known in the art and are commercially available from a number of suppliers such as Warner-Jenkinson Company, Inc. Examples of flavoring agents include peppermint extract, leaf power or oil; spearmint extract, leaf powder or oil; wintergreen oil; vanilla extract; parsley; oregano oil; bay leaf oil; clove oil; sage oil; sassafras oil; lemon oil; orange oil; anise oil; benzaldehyde; almond oil; camphor; cedar leaf oil; marjoram oil; citronella oil; lavender oil; mustard oil; pine oil; pine needle oil; rosemary oil; thyme oil; cinnamon leaf oil; menthol; carvone; anethole; eugenol; methyl salicylate; limonene; cymene; n-decyl alcohol; citronellol; α-terpineol; methyl acetate; citronellyl acetate; methyl eugenol; cineole; linalool; eyktl linalool; vanillin; thymol; pellira oil; gaultheria oil; eucalyptus oil; caffeine, cream of tartar, lactic acid, malic acid, monosodium glutamate, nitrites, sorbitol, etc. Flavoring agents are most desirable where the formulation is intended for buccal or oral administration. Flavoring agents also include sweetening agents (i.e., sweeteners) such as aspartame, acesulfame, saccharin, dextrose, levulose, sodium cyclamate, stevioside, neo-hesperidyl dihydrochalcone, glycyrrhizin, perillartine, thaumatin, aspartylphenylalanine methyl ester, p-methoxycinnamic aldehyde, etc.

Similarly, coloring agents are agents that provide color to an otherwise colorless formulation, agents that enhance a pre-existing but weak color, or agents that mask or change a pre-existing but potentially unpleasing color. Coloring agents also include agents that convert a colored formulation into a colorless one. Coloring agents are known in the art and can be purchased from the flavoring agent suppliers such as those listed above. Coloring agents may be desirable for ocular as well as oral formulation. An example of a suitable coloring agent is titanium dioxide. Suitable oral formulation coloring agents include FD&C Blue #1, FD&C Yellow #5 and #10, FD&C Red #3 and #40; caramel color or powder (#05439), chocolate shade (#05349), green lake blend (#09236), kowet titanium dioxide (#03970), yellow liquid color (#00403), and nitrites.

Scenting agents are agents that provide scent (i.e., fragrance) to an otherwise odorless formulation, agents that enhance a pre-existing but weak scent, or agents that mask or change a pre-existing but potentially unpleasing odor. Scenting agents also include agents that convert an odored formulation into an odorless one. Scenting agents are known in the art and can be purchased from the flavoring agent suppliers such as those listed above. Examples of scenting agents include natural scenting agents such as extracts of flower, herb, blossom or plant, and artificial scenting agents. Scenting agents may be desirable for ocular as well as oral formulation.

Individuals skilled in the art will recognize that modifications to these formulations can be readily made. It is to be understood that other components can be added into the formulations of the invention, including components that are themselves therapeutic or beneficial to the subject. For example, the oral formulations of the invention may include vitamins or fluoride, and the ocular formulations may include therapeutic agents such as anti-glaucoma agents, as are known in the art.

For administration by inhalation, the compounds for use according to the present invention may be conveniently delivered in the form of an aerosol spray, from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Techniques for preparing aerosol delivery systems are well known to those of skill in the art. Generally, such systems should utilize components which will not significantly impair the biological properties of the therapeutic, such as the immunostimulatory capacity of the nucleic acids (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation. Capsules and cartridges of e.g. gelatin for use in an inhaler or insufflator may be formulated containing a powder mix of the compound and a suitable powder base such as lactose or starch. Compounds to be administered to the nasal cavity can also be formulated as gels or nasal drops.

Topical administration includes administration to a skin surface and a mucosal surface. The compounds may be provided in any standard formulation that is suitable for the external surface and thus which is of a non-liquid but rather cream consistency. Mucosal surface delivery can be effected via lipsticks, lip treatments such as lip balms, lip sticks, cold sore ointments; sunscreen ointments; oral gels such as those used for mouth sores (e.g., radiation or chemotherapy induced mouth sores); toothpaste; inhalants; surface patches; and the like. If the compounds are intended for the skin, they may be provided in an ointment, a lotion, a gel, etc. As another example, if the compounds are intended for the scalp, they may be provided in a shampoo, gel or mousse, etc. For application to the nails, the compounds can be provided in hand lotions or nail lotions.

The compounds may also be formulated in rectal or vaginal compositions such as suppositories or retention enemas, e.g., containing conventional suppository bases such as cocoa butter or other glycerides. Vaginal creams or ointments can also be used. Mucosal administration can also be performed using mucoadhesive films onto which the oil-in-water emulsions are coated.

The compositions may also be delivered as a coating on administration devices such as a birth control device (e.g., a condom).

Pharmaceutical formulations for parenteral administration, such as those for delivery of the other therapeutic agents, include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Another suitable compound for sustained release delivery is GELFOAM, a commercially available product consisting of modified collagen fibers.

Compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Alternatively, compounds may be in powder form for constitution with a suitable vehicle, e.g., sterile pyrogen-free water, before use.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The pharmaceutical compositions of the invention contain an effective amount of an emulsion/nucleic acid formulation optionally included in a pharmaceutically-acceptable carrier. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The emulsion/nucleic acid formulation may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The present invention is further illustrated by the following Examples, which in no way should be construed as further limiting.

### EXAMPLES

These examples demonstrate a comparison of oil-in-water and water-in-oil formulations that contain an immunostimulatory nucleic acid in a genital herpes model.

### Example 1

Three formulations, an oil-in-water emulsion, a water-in-oil emulsion and an aqueous gel, were prepared and used to evaluate the properties of SEQ ID NO:150 immunostimulatory nucleic acid against genital herpes. Each formulation provides different cosmetic properties as well as different delivery approaches. Tables 1, 2 and 3 show the formula composition of these formulations as well as their respective manufacturing process.

Prior to formulation preparation, 2 vials containing 100 mg of SEQ ID NO:150 (Lot No. APJ-02C-001-M) were combined and diluted with purified water. The concentration of SEQ ID NO:150 was measured to be 23.31 mg/ml (2.331% w/w). The sample was then stored at 5° C. until the preparation of the following formulations.

TABLE 1
SEQ ID NO: 150 in Water-In-Oil Emulsion

| | % w/w | | | |
|---|---|---|---|---|
| Excipients | 1127-6A | 1127-13A | 1127-14A | 1127-14B |
| CpG SEQ ID NO: 150 | — | 10.0 | 1.0 | 0.1 |
| Solution, 2.3%¹ | | | | |
| White Petrolatum | 5.0 | 5.0 | 5.0 | 5.0 |
| White Wax | 5.0 | 5.0 | 5.0 | 5.0 |
| Mineral Oil | 16.0 | 16.0 | 16.0 | 16.0 |
| PEG-22 Dodecyl | 3.0 | 3.0 | 3.0 | 3.0 |
| Glycol Copolymer | | | | |
| Caprylic/Capric | 5.0 | 5.0 | 5.0 | 5.0 |
| Triglyceride | | | | |
| Sorbitan Monooleate | 3.0 | 3.0 | 3.0 | 3.0 |
| Purified Water | 62.3 | 52.3 | 61.3 | 62.2 |
| Methylparaben | 0.17 | 0.17 | 0.17 | 0.17 |
| Propylparaben | 0.03 | 0.03 | 0.03 | 0.03 |
| Magnesium Sulfate | 0.5 | 0.5 | 0.5 | 0.5 |

Note:
The 0.2% Cream (1127-13A) was prepared as follows: (1127-14A originated from a 10% dilution of 1127-13A with 1127-6A. 1127-14B originated form a 10% dilution of 1127-14A with 1127-6A.

The above formulations were prepared as follows:
1. In a manufacturing vessel, weigh PEG-22 Dodecyl Glycol Copolymer, Caprilic/Capric Triglyceride, Sorbitan Monooleate, Mineral Oil, White Wax and White Petrolatum.
2. In a separate container, add purified water, Methylparaben, Propylparaben and Magnesium Sulfate. Agitate mixture until solution is achieved.
3. Heat step 1 and step 2 to 75±5° C.
4. Add step 2 to step 1. Utilizing a rotor stator, agitate mixture until homogeneous emulsion is achieved.
5. With continuous mixing allow step 4 to cool down to temperatures below 40° C.
6. With continuous mixing add 2.3% CpG Solution to step 5. Continue mixing until a homogeneous system is achieved and temperatures below 30° C. are reached.

TABLE 2
SEQ ID NO: 150 in Oil-In-Water Emulsion

| | % w/w | | | |
|---|---|---|---|---|
| Excipients | 1127-9A | 1127-15A | 1127-16A | 1127-16B |
| CpG SEQ ID NO: 150 | — | 10.0 | 1.0 | 0.1 |
| Solution, 2.3%¹ | | | | |
| White Petrolatum | 10.0 | 10.0 | 10.0 | 10.0 |
| Stearyl Alcohol | 5.0 | 5.0 | 5.0 | 5.0 |
| Steareth 21 | 1.0 | 1.0 | 1.0 | 1.0 |
| Steareth 2 | 1.2 | 1.2 | 1.2 | 1.2 |
| Purified Water | 77.4 | 67.4 | 76.4 | 77.3 |
| Glycerin | 5.0 | 5.0 | 5.0 | 5.0 |
| Methylparaben | 0.17 | 0.17 | 0.17 | 0.17 |
| Propylparaben | 0.03 | 0.03 | 0.03 | 0.03 |
| Carbopol 981 | 0.1 | 0.1 | 0.1 | 0.1 |
| 10% Sodium Hydroxide | 0.1 | 0.1 | 0.1 | 0.1 |
| Solution | | | | |

Note:
The 0.2% Cream (1127-15A) was prepared as follows: (1127-16A originated from a 10% dilution of 1127-15A with 1127-9A. 1127-16B originated form a 10% dilution of 1127-16A with 1127-9A.

The formulations of Table 2 were prepared as follows:
1. In a manufacturing vessel weigh Purified Water, Glycerin, Methylparaben, and Propylparaben. Agitate mixture until solution is achieved.
2. With continuous propeller mixing, disperse Carbopol 981 into step 1. Continue mixing until polymer is polymer is properly hydrated.
3. In a separate container add Stearyl Alcohol, Steareth 21, Steareth 2 and White Petrolatum.
4. Heat step 2 and step 3 to 75±5° C.
5. Add step 3 to step 2. Utilizing a rotor stator, agitate mixture until homogeneous emulsion is achieved.
6. With continuous mixing allow step 4 to cool down to temperatures below 40° C.
7. With continuous mixing add 2.3% CpG Solution to step 6.
8. With continuous mixing add 10% Sodium Hydroxide Solution to step 7. Continue mixing until a homogeneous system is achieved and temperatures below 30° C. are reached.

TABLE 3
SEQ ID NO: 150 in an Aqueous Gel

| | % w/w | | | |
|---|---|---|---|---|
| Excipients | 1127-12A | 1127-18A | 1127-19A | 1127-19B |
| CpG SEQ ID NO: 150 | — | 10.0 | 1.0 | 0.1 |
| Solution, 2.3%¹ | | | | |
| Purified Water | 62.7 | 52.7 | 61.7 | 62.6 |
| Glycerin | 10.0 | 10.0 | 10.0 | 10.0 |
| Methylparaben | 0.25 | 0.25 | 0.25 | 0.25 |
| Propylparaben | 0.05 | 0.05 | 0.05 | 0.05 |
| 200 mM Phosphate | 25.0 | 25.0 | 25.0 | 25.0 |
| Buffer | | | | |
| Hydroxyethylcellulose, | 2.0 | 2.0 | 2.0 | 2.0 |
| 250 HHX | | | | |

Note:
The 0.2% Gel (1127-18A) was prepared as follows: (1127-19A originated from a 10% dilution of 1127-18A with 1127-12A. 1127-19B originated form a 10% dilution of 1127-19A with 1127-12A.

The formulations of Table 3 were prepared as follows:
1. In a manufacturing vessel weigh Purified Water, Glycerin, Methylparaben, Propylparaben and 200 mM Phosphate Buffer. Agitate mixture until solution is achieved.
2. With continuous mixing add 2.3% CpG Solution to step 1.
3. With continuous mixing, disperse Hydroxyethylcellulose, 250HHX into step 2.

Continue mixing until homogeneous gel is formed.

Nucleic acid SEQ ID NO:150 appears to be physically stable with the systems evaluated. No signs of precipitation or chemical incompatibilities were noticed throughout the manufacturing processes. The pH of the active finish products were not measured due to their limited availability. The vehicles for the gel and for the oil-in-water emulsion maintained a relatively neutral pH of 6.0, while the pH of the water-in-oil emulsion could not be measured due to the products inherent properties.

### Example 2

Methods: Mice were challenged 5 days after progesterone Rx (i.e., in diestrus) by intravaginal delivery of 10 μl containing 10⁴ PFU HSV-2 (strain 333).

Mice were then administered one of the following formulations:

1. CpG immunostimulatory nucleic acid (TCG TCG TTT CGT CGT TTT GTC GTT; SEQ ID NO:150) in saline;

2. CpG immunostimulatory nucleic acid (TCG TCG TTT CGT CGT TTT GTC GTT; SEQ ID NO:150) in water-in-oil emulsion (cream consistency);

3. CpG immunostimulatory nucleic acid (TCG TCG TTT CGT CGT TTT GTC GTT; SEQ ID NO:150) in oil-in-water emulsion (cream consistency); and

4. controls formulations that contain cream alone.

The treatment schedule was either a single dose of 100 μg nucleic acid administered intravaginally 4 hours after challenge with HSV-2, or in multiple doses of either 10 μg or 100 μg nucleic acid administered intravaginally 4 hours after challenge with HSV-2, and then daily thereafter for a total of 5 days.

The mice were evaluated for pathology scores (on a daily basis) and survival time was followed for 15 days.

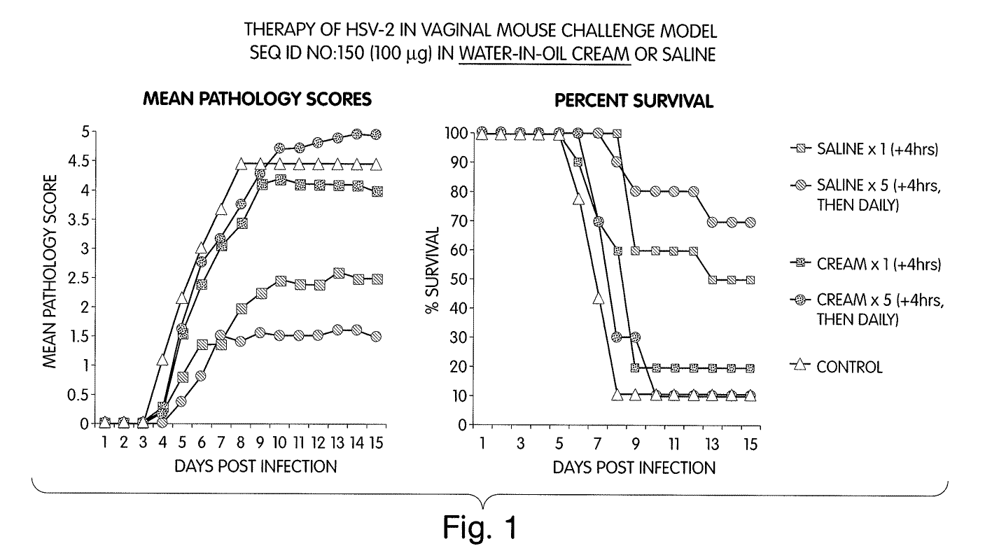

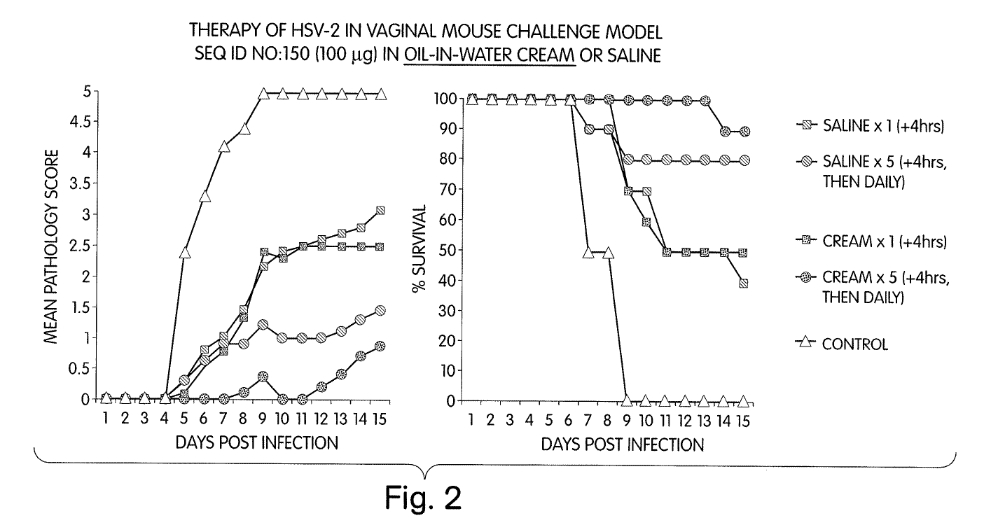

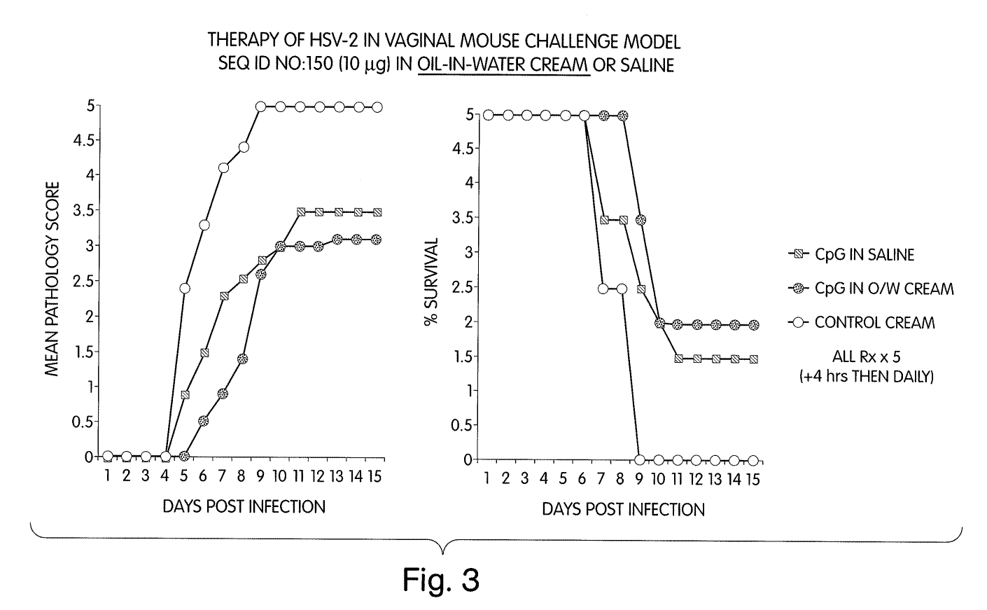

Results: The results are shown in FIGS. 1-3. The water-in-oil formulation was no better than control treatments. This suggests that the nucleic acid, which would be in the water droplets surrounded in the oil, could not contact or be transferred across the mucosal membrane due to the presence of the oil barrier.

The oil-in-water formulation was in most instances better than nucleic acid in a saline formulation, and in all instances better than the nucleic acid in the water-in-oil formulation. This suggests that the nucleic acid, which would be in an aqueous phase would have contact with a large surface area of mucosa, allowing the nucleic acid to cross into the membrane similar to a saline solution. This formulation may also be improved because the cream carrier holds the nucleic acid at a localized area better than does a saline solution.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A method for inducing an immune response, comprising:
topically administering to a subject an oil-in-water emulsion and an immunostimulatory nucleic acid in an effective amount to induce an immune response.

2. The method of claim 1, wherein the immune response is an antigen specific immune response.

3. The method of claim 1, further comprising administering an antigen.

4. The method of claim 1, wherein the immunostimulatory nucleic acid is a CpG immunostimulatory nucleic acid.

5. (canceled)

6. The method of claim 1, wherein the subject has an infectious disease.

7-8. (canceled)

9. The method of claim 6, wherein the infectious disease is a viral infection.

10. (canceled)

11. The method of claim 1, wherein the oil-in-water emulsion and the immunostimulatory nucleic acid is administered to the skin.

12. The method of claim 1, wherein the oil-in-water emulsion and the immunostimulatory nucleic acid is administered to a mucosal surface.

13-21. (canceled)

22. The method of claim 1, wherein the immunostimulatory nucleic acid has a modified backbone.

23. The method of claim 22, wherein the modified backbone is a phosphate modified backbone.

24. The method of claim 23, wherein the phosphate modified backbone is a phosphorothioate modified backbone.

25. The method of claim 23, wherein the modified backbone is a peptide modified oligonucleotide backbone.

26. The method of claim 1, wherein the subject is an immunocompromised subject.

27. The method of claim 1, wherein the immunostimulatory nucleic acid has the nucleotide sequence of TCG TCG TTT CGT CGT TTT GTC GTT (SEQ ID NO: 150).

28-35. (canceled)

36. The method of claim 1, wherein the immune response is an innate immune response.

37. The method of claim 1, wherein the immune response is an adaptive immune response.

38. The method of claim 1, wherein the immune response is a local immune response.

39. The method of claim 1, wherein the subject is actively exposed to an antigen.

40. The method of claim 1, wherein the subject is passively exposed to an antigen.

41. A composition comprising
an immunostimulatory nucleic acid and an oil-in-water emulsion, formulated for topical skin or mucosal delivery.

42-61. (canceled)
